(19) United States
(12) Patent Application Publication
(10) Pub. No.: US 2023/0136562 A1
(43) Pub. Date: May 4, 2023

(54) SPIROUREA DERIVATIVES

(71) Applicant: Idorsia Pharmaceuticals Ltd, Allschwil (CH)

(72) Inventors: Olivier BEZENCON, Allschwil (CH); Caroline DEYMIER, Allschwil (CH); Jens-Uwe PETERS, Allschwil (CH); Romain SIEGRIST, Allschwil (CH); Jean-Philippe SURIVET, Allschwil (CH)

(21) Appl. No.: 17/997,483

(22) PCT Filed: Apr. 27, 2021
(86) PCT No.: PCT/EP2021/060918

§ 371 Date: Oct. 28, 2022

(30) Foreign Application Priority Data
Apr. 29, 2020 (WO) PCT/EP2020/061911

(51) Int. Cl. C07C 275/26; C07D 213/16; C07D 217/26; C07D 239/26; C07D 257/04; C07D 255/02; C07C 317/28

(52) U.S. Cl. CPC C07C 275/26; C07D 213/16; C07D 217/26; C07D 239/26; C07D 257/04; C07D 255/02; C07C 317/28

(57) ABSTRACT

The invention relates to compounds of Formula (I)

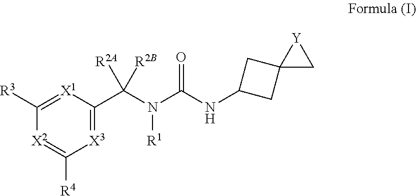

wherein X¹, X², X³, Y, R¹, R^{2A}, R^{2B}, R³, and R⁴ are as described in the description; to their preparation, to pharmaceutically acceptable salts thereof, to pharmaceutical compositions containing one or more compounds of Formula (I), and to the use of such compounds as medicaments, especially as Kv7 openers.

The present invention relates to novel spirourea derivatives of Formula (I), and their use as pharmaceuticals. The invention also concerns related aspects including processes for the preparation of the compounds, pharmaceutical compositions containing one or more compounds of Formula (I), and especially their use as Kv7 potassium channel openers.

Kᵥ channels are composed of tetramers of α-subunits. Each α-subunit consists of six transmembrane α-helical structures (S1-S6), with an intracellular localization of both the NH₂ and COOH termini. The regions S1-S4 constitute the voltage-sensing domain, whereas the S5 and S6 regions and their linker form the ion-selective pore. Additionally, ancillary proteins are either cytosolic subunits or transmembrane subunits (β).

The Kᵥ7 family comprises five α-subunit Kᵥ7.1-5, encoded by the genes KCNQ1-5. These α-subunits are arranged as homotetramers (Kᵥ7.1, Kᵥ7.2, or Kᵥ7.4) or heterotetramers (Kᵥ7.2/3, Kᵥ7.3/5, or Kᵥ7.4/5). Kᵥ7.1 is mainly localized in cardiomyocytes, gastrointestinal epithelium, skeletal muscles, vascular smooth muscles and the inner ear. In cardiomyocytes, they slowly activate I_{KS} current which plays a central role in ventricular repolarization. Kv7.2-Kv7.5 are widely expressed in neuronal tissue with Kᵥ7.2 and Kᵥ7.3 playing a dominant role and found as Kv7.2 homotetramer or Kv7.2/7.3 and Kv7.3/7.5 heterotetramers. They underlie the M-current, which stabilizes the resting membrane potential and reduces action potential firing. Kv7.4 is expressed in outer hair cells and in neurons of the central auditory pathway nuclei. Kv7.4 and Kv7.5 are both also widely expressed in visceral, vascular and airway smooth muscle, skeletal muscle as Kv7.4 homotetramer or Kv7.4/7.5 heterotetramer. They control auditory physiology and contractility of smooth muscle cells notably. Finally, Kv7.5 is only found in heterotetramers, as discussed previously (Miceli et al. Curr. Med. Chem., 2018, 25, 2637-2660).

Activation of the Kv7 channels occurs at potential around −60 mV and results in potassium efflux and membrane hyperpolarization. Dysfunctions or mutations in the Kv7 channels can result physiologically in various channelopathies (C. Bock, A. Link, Future Med. Chem. 2019, 11, 337-355). The neuronal Kv7 channels are responsible for the M-current which regulates neuronal excitability. Due to the dominant role of the M-current in controlling action potential firing, Kv7 openers might be a potential therapy in diseases where enhanced neuronal excitability is a significant aspect of the pathology, such as epilepsy, myokymia, tinnitus, neuropathic and inflammatory pain and psychiatric disorders such as anxiety, schizophrenia, mania, autism (Maljevic et al, 2008, J Physiol 586(7): 1791-1801; Diao et al, 2017, Neuropsychiatry 7(1): 26-31; Rivera-Arconada et al., 2017, Oncotarget 8(8): 12554-12555; Maljevic et al, 2010, Pflugers Arch 460(2):277-88). Due to the wide distribution of Kᵥ7 channels in other tissues Kᵥ7 openers might be also useful in diseases affecting the visceral smooth muscles such as functional dyspepsia, irritable bowel syndrome and overactive bladder, in diseases affecting the vascular smooth muscles such as hypertension and cerebral vasospasm, in diseases affecting the airway smooth muscles such as asthma and chronic obstructive pulmonary disease and in hearing disorder (Haick and Byron 2016, Pharmacol Ther 165: 14-25; Fosmo and Skraastad 2017, Front Cardiovasc Med 4: 75).

In addition, Kᵥ7 openers might be a potential therapy in disorders associated with KCNQ2, KCNQ3, KCNQ4, KCNQ5 and disorders associated with mutations in KCNQ2, KCNQ3, KCNQ4, KCNQ5 (Dedek, Kunath et al. 2001, Proc Natl Acad Sci USA 98(21): 12272-12277; Wuttke, Jurkat-Rott et al. 2007, Neurology 69(22): 2045-2053; Millichap, Park et al. 2016, Neurol Genet 2(5): e96; Allen et al 2020, Eur J Paediatr Neurol 2020; 24:105-116).

More specifically, Kᵥ7 openers are suitable antiepileptics drugs, as demonstrated with the FDA-approved drug retigabine/ezogabine. Retigabine/ezogabine activates the potassium current of the different Kᵥ7 channels by binding near the channel gate leading to a stabilization of the channel open state and to a shift of the voltage-dependence of KCNQ activation to more hyperpolarized potentials (Gunthorpe, Large et al. 2012, Epilepsia 53(3): 412-424). Retigabine/ezogabine reduces seizure activity in various rodent models including acute seizure models, genetic models of enhanced seizure sensitivity such as the audiogenic seizure-sensitive DBA2 mice showing generalized tonic-clonic seizures and models of induced epilepsy such as the rat kindling model presenting with focal onset seizures that propagate to bilateral tonic-clonic seizures (Rostock et al. 1996, Epilepsy Res 23(3): 211-223; Tober et al. 1996, Eur J Pharmacol 303(3): 163-169; De Sarro G, Di Paola E G et al. 2001, Naunyn-Schmiedeberg's Arch Pharmacol 363: 330-336). In two phase three trials retigabine/ezogabine significantly reduced seizure frequency in patients with drug-resistant focal-onset seizures (Brodie, Lerche et al. 2010, Neurology 75(20): 1817-1824; French, Abou-Khalil et al. 2011, Neurology 76(18): 1555-1563).

Moreover, mutations in KCNQ2 and KCNQ3 were recently identified in patients that had been diagnosed with epileptic encephalopathy, infantile/childhood epilepsy syndrome or neurodevelopmental disorders with epilepsy (Helbig and Tayoun 2016, Mol Syndromol 7(4): 172-181; Heyne, Singh et al. 2018, Nat Genet 50(7): 1048-1053). Knock-in mice carrying a KCNQ2 or KCNQ3 variant known to cause reduction of the wild-type potassium current and identified in patients diagnosed with an early onset epileptic syndromes show spontaneous seizures, reduced seizure thresholds, and seizures that are attenuated by retigabine/ezogabine (Singh, Otto et al. 2008, J Physiol 586(14): 3405-3423; Otto, Singh et al. 2009, Epilepsia 50(7): 1752-1759; Tomonoh, Deshimaru et al. 2014, PLoS One 9(2): e88549; Ihara, Tomonoh et al. 2016, PLoS One 11(2): e0150095; Milh, Roubertoux et al. 2020, Epilepsia, doi: 10.1111/epi.16494).

Therefore, Kᵥ7 opener might be a potential therapy in epilepsy including epilepsy with focal onset seizures with or without impaired awareness, with focal onset seizures with motor or nonmotor onset symptoms and with or without focal seizures that develop into bilateral tonic-clonic seizures. Kᵥ7 opener might be a potential therapy in epilepsy with generalized seizures with motor onset symptoms, as well as epilepsy with unknown seizure onset or epilepsy with traumatic brain injury-induced seizures (Diao et al, 2017, Neuropsychiatry 7(1): 26-31; Vigil, Bozdemir et al. 2019, J Cereb Blood Flow Metab: 271678X19857818).

Kᵥ7 opener might be a potential therapy in neonatal onset epilepsy with or without neurodevelopmental impairment including early onset epileptic encephalopathy such as Othahara syndrome or early infantile epileptic encephalopathy, early myoclonic encephalopathy and epilepsy with suppression-burst pattern, but also including benign or self-limiting familial neonatal epilepsy (Singh, Westenskow et al. 2003, Brain 126(Pt 12): 2726-2737; Weckhuysen, Mandelstam et al. 2012, Ann Neurol 71(1): 15-25; Olson, Kelly et al. 2017, Ann Neurol 81(3): 419-429; Milh, Roubertoux et al. 2020, Epilepsia, doi: 10.1111/epi.16494).

Kᵥ7 opener might be a potential therapy in infantile/childhood epilepsy syndromes including epilepsy with neurodevelopmental impairment, focal epilepsies of childhood and idiopathic epilepsy syndromes (Neubauer, Waldegger et al. 2008, Kato, Yamagata et al. 2013, Lesca and Depienne 2015, Heyne, Singh et al. 2018, Lindy, Stosser et al. 2018).

WO2019/161877 discloses alcohol derivatives which activate the Kᵥ7 potassium channels and are claimed to treat disorders responsive to the activation of Kᵥ7 potassium channels. Different cyclic amides, acetamides and ureas which are useful as potassium channel openers, have been disclosed in EP3366683A1 and WO2018/158256 and pentacyclothienyl and indanyl urea derivatives in EP3567034A1.

In the context of a phenotypic screening program aimed at identifying anticonvulsive compounds, new spirourea derivatives were identified, which were found to act pharmacologically as Kᵥ7 opener and which may be useful for the treatment of diseases which are modulated by the KCNQ potassium channels.

1) In a first embodiment, the present invention relates to compounds of Formula (I)

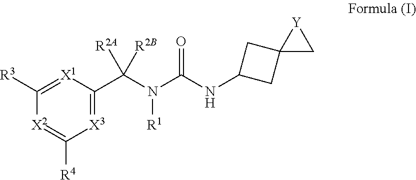

wherein

X¹ represents nitrogen or CR^{X1}; wherein R^{X1} represents hydrogen, halogen, (C₁₋₄)alkyl, or (C₁₋₄)alkoxy;

X² represents nitrogen or CR^{X2}; wherein R^{X2} represents hydrogen, halogen, (C₁₋₄)alkyl, or (C₁₋₄)alkoxy;

X³ represents nitrogen or CR^{X3}; wherein R^{X3} represents hydrogen, halogen, (C₁₋₄)alkyl, (C₁₋₄)alkoxy, or hydroxy;
R¹ represents hydrogen, or methyl; or
R¹ and R^{X1} together represent a —CH₂CH₂— bridge;

Y represents —C(R^{Y1})(R^{Y2})—, or *—CH₂—C(R^{Y3})(R^{Y4})— wherein the asterisk indicates the bond which is linked to the cyclobutyl ring;

R^{Y1} represents hydrogen, or fluoro;

R^{Y2} represents hydrogen or fluoro;

R^{Y3} represents hydrogen, fluoro or iodo;

R^{Y4} represents hydrogen or fluoro;
R^{2A} represents hydrogen; (C₁₋₄)alkyl; (C₂₋₄)alkenyl; (C₂₋₄)alkynyl; (C₃₋₆)cycloalkyl; (C₁₋₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋₂)alkyl; (C₁₋₂)alkyl-S—(C₁₋₂)alkyl; (C₁₋₂)alkyl-(SO₂)—(C₁₋₂)alkyl; cyano; (C₁₋₂)cyanoalkyl; H₂N—C(O)—(C₁₋₂)alkyl; (R^{N1})₂N—(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl;
and R^{2B} represents hydrogen, or methyl; or
R^{2A} and R^{2B} form, together with the carbon atom to which they are attached, a ring of 3- to 6 members, wherein the members needed to complete said ring are each independently selected from —CH₂— and —O— and wherein said ring does not contain more than one —O— member; or
R^{2A} and R^{X3} together represent a —CH₂—O—* bridge, wherein the asterisk indicates the bond which is linked to the aromatic carbon atom, and
R^{2B} represents hydrogen;

R³ represents
SF₅;
((C₁₋₂)alkyl-S(O)₂—; or
a fragment

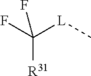

wherein
   R³¹ represents hydrogen, or fluoro; and
   L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋₄)alkyl (especially methyl), CH₃—O—CH₂—, or (CH₃)₂NCH₂—; wherein the asterisks indicate the bond which is linked to the aromatic carbon atom; or

R³ and R^{X1} together represent a —O—CF₂—O— bridge; or

R³ and R^{X2} together represent a —O—CF₂—O— bridge; and

R⁴ represents hydrogen, halogen (especially fluoro, chloro), or (C₁₋₄)alkyl (especially methyl); and to the salts (in particular pharmaceutically acceptable salts) of such compounds.

Definitions provided herein are intended to apply uniformly to the compounds of Formula (I) as defined in any one of embodiments 1) to 37), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The compounds of Formula (I) as defined in any one of embodiments 1) to 37), may contain one or more stereogenic or asymmetric centers, such as one or more asymmetric carbon atoms. The compounds of Formula (I) may thus be present as mixtures of stereoisomers or in stereoisomerically enriched form, preferably as pure stereoisomers. Mixtures of stereoisomers may be separated in a manner known to a person skilled in the art.

The term “enriched”, for example when used in the context of enantiomers, is understood in the context of the present invention to mean especially that the respective enantiomer is present in a ratio (mutatis mutandis:purity) of at least 70:30, and notably of at least 90:10 (mutatis mutandis:purity of 70%/90%) with respect to the respective other enantiomer. Preferably the term refers to the respective essentially pure enantiomer. The term “essentially”, for example when used in a term such as “essentially pure” is understood in the context of the present invention to mean especially that the respective stereoisomer/composition/compound etc. consists in an amount of at least 90, especially of at least 95, and notably of at least 99 percent by weight of the respective pure stereoisomer/composition/compound etc.

Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and/or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s)/is a carbocycle/or the like), or the respective optional heteroatom(s), or the like, are present as explicitly defined.

In this patent application, a dotted line shows the point of attachment of the radical drawn. For example, the radical

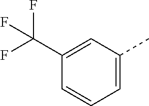

is a 3-(trifluoromethyl)phenyl group.

The term “halogen” means fluorine, chlorine, or bromine, preferably fluorine or chlorine. In case R^{X1} or R^{X2} represents halogen the term means preferably a fluoro- or chloro-substituent; in case of R^{X3} the term means preferably a fluoro-, chloro-, or bromo-substituent. In case of R⁴ representing halogen, the term preferably refers to a fluoro- or chloro-substituent.

The term “alkyl”, used alone or in combination, refers to a straight or branched saturated hydrocarbon chain containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, n-propyl, iso-propyl, n-butyl, iso-butyl, sec.-butyl and tert.-butyl. Examples of (C₁₋₂)alkyl groups are methyl and ethyl. In case R^{2A} represents “(C₁₋₄)alkyl” the term means preferably methyl. In case R⁴, R^{L}, R^{X1}, R^{X2}, or R^{X3} represents “(C₁₋₄)alkyl” the term means preferably methyl.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O— in which the term “(C₁₋₄)alkyl” has the previously given significance. Examples of (C₁₋₄)alkoxy groups are methoxy, ethoxy, n-propoxy, iso-propoxy, n-butoxy, iso-butoxy, sec.-butoxy and tert.-butoxy. Examples of (C₁₋₂)alkoxy groups are methoxy and ethoxy. In case R^{X1}, R^{X2}, or R^{X3} represents “(C₁₋₄)alkoxy” the term preferably means methoxy.

The term “(C_{xa-ya})alkoxy-(C_{x-y})alkyl” (x, xa, y and ya each being an integer) refers to an alkyl group as defined before wherein one hydrogen atom has been replaced by (C_{xa-ya})alkoxy as defined before containing xa to ya carbon atoms. In case R^{2A} represents “(C₁₋₄)alkoxy-(C₁₋₂)alkyl” the term means preferably methoxymethyl, ethoxyethyl and iso-propoxymethyl and more preferably methoxymethyl.

The term “(C_{xa-ya})alkoxy-(C_{x-y})alkoxy” (x, xa, y and ya each being an integer) refers to an alkoxy group as defined before containing x to y carbon atoms wherein one hydrogen atom has been replaced with (C_{xa-ya})alkoxy as defined before containing xa to ya carbon atoms. For example a “(C₁₋₂)alkoxy-(C₁₋₂)alkoxy group” refers to an (C₁₋₂)alkoxy group as defined before containing one or two carbon atoms wherein one hydrogen atom has been replaced with (C₁₋₂)alkoxy as defined before containing one or two carbon atoms. It is preferred that the oxygen-atom of the (C_{x-y})alkoxy group and the oxygen atom of the (C_{xa-ya})alkoxy group are attached to different carbon-atoms of the (C_{x-y})alkoxy group. Representative examples of (C₁₋₂)alkoxy-(C₁₋₂)alkoxy groups include methoxy-methoxy, 2-methoxy-ethoxy, ethoxy-methoxy, and 2-ethoxy-ethoxy.

The term “(C_{xa-ya})alkoxy-(C_{xb-yb})alkoxy-(C_{x-y})alkyl” (x, xa, xb, y, ya and yb each being an integer) refers to an alkyl group as defined before wherein one hydrogen atom has been replaced by (C_{xa-ya})alkoxy-(C_{xb-yb})alkoxy as defined before. In case R^{2A} represents “(C₁₋₂)alkoxy-(C₁₋₂)alkoxy-(C₁₋₂)alkyl” the term means preferably 2-methoxy-ethoxy-methyl.

The term “(C₁₋₄)fluoroalkyl” refers to an alkyl group as defined before containing one to four carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₄)fluoroalkyl group contains from one to four carbon atoms in which one to nine hydrogen atoms have been replaced with fluorine. Representative examples of (C₁₋₄)fluoroalkyl groups include difluoromethyl, trifluoromethyl, 2-fluoroethyl, 2,2-difluoroethyl, and 2,2,2-trifluoroethyl. Preferred are (C₁)fluoroalkyl groups such as trifluoromethyl or difluoromethyl. In case R^{2A} represents “(C₁₋₄)fluoroalkyl” the term means preferably trifluoromethyl, difluoromethyl and 2,2-difluoroethyl.

The term “cycloalkyl”, used alone or in combination, refers to a saturated carbocyclic ring containing three to six carbon atoms. The term “(C_{x-y})cycloalkyl” (x and y each being an integer), refers to a cycloalkyl group as defined before containing x to y carbon atoms. For example a (C₃₋₆)cycloalkyl group contains from three to six carbon atoms. Examples of cycloalkyl groups are cyclopropyl, cyclobutyl, cyclopentyl, and cyclohexyl. In case R^{2A} represents “(C₃₋₆)cycloalkyl” the term means preferably cyclopropyl.

The term “alkenyl”, used alone or in combination, refers to a straight or branched hydrocarbon chain containing two to five carbon atoms and one carbon-carbon double bond. The term “(C_{x-y})alkenyl” (x and y each being an integer), refers to an alkenyl group as defined before containing x to y carbon atoms. For example a (C₂₋₄)alkenyl group contains from two to four carbon atoms. Representative examples of “(C₂₋₄)alkenyl” group are vinyl, prop-1-en-1-yl, prop-2-en-1-yl, but-2-en-1-yl, but-1-en-1-yl, and but-3-en-1-yl. In case R^{2A} represents “(C₂₋₄)alkenyl” the term means preferably prop-2-en-1-yl.

The term “alkynyl”, used alone or in combination, refers to a straight or branched chain hydrocarbon group containing one to six (especially one to four) carbon atoms wherein said hydrocarbon group contains at least one carbon-carbon triple bond. The term “(C_{x-y})alkynyl” (x and y each being an integer), refers to an alkynyl group as defined before, containing x to y carbon atoms. For example a (C₂₋₄)alkynyl group contains from two to four carbon atoms. Representative examples of “(C₂₋₄)alkynyl” group are ethynyl, prop-1-yn-1-yl, prop-2-yn-1-yl, but-2-yn-1-yl, but-1-yn-1-yl, and but-3-yn-1-yl.

The term “cyano” refers to a group —CN.

The term “(C_{x-y})cyanoalkyl” (x and y each being an integer) refers to an alkyl group as defined before containing x to y carbon atoms wherein one hydrogen atom has been replaced by a cyano group. Representative examples of “(C₁₋₂)cyanoalkyl” are cyanomethyl and 2-cyanoethyl. In case R^{2A} represents “(C₁₋₂)cyanoalkyl” the term means preferably cyanomethyl.

—(SO₂)— refers to a sulfonyl group and —C(O)— refers to a carbonyl group. In case R^{2A} represents “(C₁₋₂)alkyl-(SO₂)—(C₁₋₂)alkyl” the term means preferably methylsulfonyl-methyl and 2-methylsulfonylethyl.

In case R^{2A} represents “(C₁₋₂)alkyl-S—(C₁₋₂)alkyl” the term means preferably 2-methylthio-ethyl.

In case R^{2A} represents “H₂N—C(O)—(C₁₋₂)alkyl” the term means preferably 3-amino-3-oxopropyl; “(R^{N1})₂N—(C₁₋₂)alkyl” means preferably dimethylamino-methyl; and “(R^{N1})₂N—C(O)—” means preferably aminocarbonyl, and methylamino-carbonyl.

In case R^{2A} represents “(C₁₋₄)hydroxyalkyl” the term means preferably hydroxymethyl, hydroxyethyl and 2-hydroxyprop-2-yl.

The term “heteroaryl”, used alone or in combination, refers to a heteroaryl-group as specifically defined which group may be unsubstituted or substituted as specifically defined.

Examples of “5-membered heteroaryl group containing one to four nitrogen atoms” are pyrrolyl, imidazolyl, pyrazolyl, triazolyl, tetrazolyl. Preferred examples of R^{2A} representing 5-membered heteroaryl group containing one to four nitrogen atoms are triazolyl and tetrazolyl; in particular 1H-1,2,4-triazol-3-yl, 1H-1,2,3-triazol-4-yl, 1H-1,2,3-triazol-5-yl, 2H-tetrazol-5-yl, and 1H-tetrazol-5-yl. Said 5-membered heteroaryl groups are unsubstituted or substituted as explicitly defined.

Preferred examples where “R^{2A} and R^{2B} form, together with the carbon atom to which they are attached, a ring of 3- to 6 members, wherein the members needed to complete said ring are each independently selected from —CH₂— and —O— and wherein said ring does not contain more than one —O— member” are cycloprop-1,1-diyl, cyclobut-1,1-diyl, oxetane-3,3-diyl and tetrahydropyran-4,4-diyl.

The term “oxo” refers to a group ═O which is preferably attached to a chain or ring carbon atom as for example in a carbonyl group —C(O)—.

Whenever two substituents together represent a “bridge”, it is to be understood that the atoms to which said substituents are attached, are connected via a —CH₂CH₂—, —CH₂—O—, or —O—CF₂—O— bridge as explicitly defined.

2) A second embodiment relates to compounds according to embodiment 1), wherein R¹ represents hydrogen; and to the salts (in particular pharmaceutically acceptable salts) of such compounds.

3) Another embodiment relates to compounds according to any one of embodiments 1) or 2), wherein Y represents —C(R^{Y1})(R^{Y2})—; and to the salts (in particular pharmaceutically acceptable salts) of such compounds.

4) Another embodiment relates to compounds according to any one of embodiments 1) or 2), wherein Y represents *—CH₂—C(R^{Y3})(R^{Y4})— wherein the asterisk indicates the bond which is linked to the cyclobutyl ring; and to the salts (in particular pharmaceutically acceptable salts) of such compounds.

5) Another embodiment relates to compounds according to any one of embodiments 1) to 4), wherein R^{Y1}, R^{Y2}, R^{Y3} and R^{Y4} all represent hydrogen; and to the salts (in particular pharmaceutically acceptable salts) of such compounds.

6) Another embodiment relates to compounds according to any one of embodiments 1) to 4), wherein R^{Y1}, R^{Y2}, R^{Y3} and R^{Y4} all represent fluoro; and to the salts (in particular pharmaceutically acceptable salts) of such compounds.

7) Another embodiment relates to compounds according to any one of embodiments 1) to 6), wherein
R^{2A} represents hydrogen, (C₁₋₄)alkyl, (C₃₋₆)cycloalkyl, (C₁₋₄)fluoroalkyl, (C₁₋₄)hydroxyalkyl, (C₁₋₄)alkoxy-(C₁₋₂)alkyl, (C₁₋₂)alkyl-S—(C₁₋₂)alkyl, (C₁₋₂)alkyl-(SO₂)—(C₁₋₂)alkyl, cyano, (C₁₋₂)cyanoalkyl, H₂N—C(O)—(C₁₋₂)alkyl, (R^{N1})₂N—(C₁₋₂)alkyl, or (R^{N1})₂N—C(O)—, wherein R^{N1} independently represents hydrogen or (C₁₋₂)alkyl;
and R^{2B} represents hydrogen; or
R^{2A} and R^{2B} form, together with the carbon atom to which they are attached, a ring of 3- to 6 members selected from cycloprop-1,1-diyl, cyclobut-1,1-diyl, oxetane-3,3-diyl and tetrahydropyran-4,4-diyl;

and to the salts (in particular pharmaceutically acceptable salts) of such compounds.

8) Another embodiment relates to compounds according to any one of embodiments 1) to 6), wherein

R^{2A} represents hydrogen, (C₁₋₄)alkyl, or (C₁₋₄)hydroxyalkyl;

and R^{2B} represents hydrogen;

and to the salts (in particular pharmaceutically acceptable salts) of such compounds.

9) Another embodiment relates to compounds according to any one of embodiments 1) to 6), wherein R^{2A} represents hydrogen, methyl, or hydroxymethyl (especially hydrogen or hydroxymethyl); and R^{2B} represents hydrogen;

and to the salts (in particular pharmaceutically acceptable salts) of such compounds.

10) Another embodiment relates to compounds according to any one of embodiments 1) to 9), wherein R³ represents a fragment

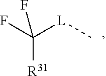

wherein

R³¹ represents hydrogen, or fluoro; and

L represents a direct bond, —CHR^{L}—O—*, —O—CH₂—*, —CH₂—NH—*, —CH₂—N(CH₃)—*, or —O—; wherein R^{L} represents hydrogen, methyl, CH₃—O—CH₂—, or (CH₃)₂NCH₂—; wherein the asterisks indicate the bond which is linked to the aromatic carbon atom;

and to the salts (in particular pharmaceutically acceptable salts) of such compounds.

11) Another embodiment relates to compounds according to any one of embodiments 1) to 9), wherein R³ represents a fragment

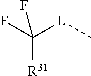

wherein

R³¹ represents fluoro; and

L represents a direct bond, —CH₂—O—*, or —CH(CH₃)—O—*; wherein the asterisks indicate the bond which is linked to the aromatic carbon atom;

and to the salts (in particular pharmaceutically acceptable salts) of such compounds.

12) Another embodiment relates to compounds according to any one of embodiments 1) to 11), wherein R⁴ represents hydrogen; and to the salts (in particular pharmaceutically acceptable salts) of such compounds.

13) Another embodiment relates to compounds according to any one of embodiments 1) to 12), wherein R^{X1}, R^{X2}, and R^{X3} all represent hydrogen; and to the salts (in particular pharmaceutically acceptable salts) of such compounds.

14) Another embodiment relates to compounds according to any one of embodiments 1) to 12), wherein the fragment

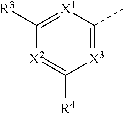

represents:

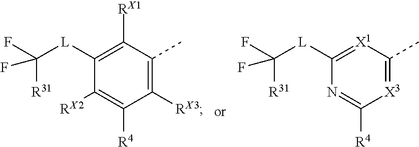

wherein
X³ represents nitrogen and X¹ represents CR^{X1};
X¹ represents nitrogen and X³ represents CR^{X3}; or
X¹ represents CR^{X1} and X³ represents CR^{X3};

and to the salts (in particular pharmaceutically acceptable salts) of such compounds.

15) Another embodiment relates to compounds according to any one of embodiments 1) to 9), wherein the fragment

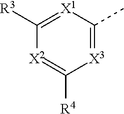

represents:

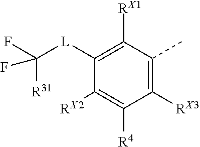

wherein R^{X1} represents hydrogen, fluoro, chloro, methyl, or methoxy; R^{X2} represents hydrogen, fluoro, chloro, methyl, or methoxy; R^{X3} represents hydrogen, fluoro, chloro, bromo, hydroxy or methoxy; R⁴ represents hydrogen, fluoro, chloro, or (C₁₋₄)alkyl (especially methyl); R³¹ represents hydrogen, or fluoro; and L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, or methyl; wherein the asterisks indicate the bond which is linked to the aromatic carbon atom; or

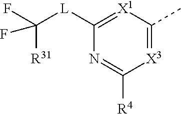

wherein
   X³ represents nitrogen and X¹ represents CR^{X1};
   X¹ represents nitrogen and X³ represents CR^{X3}; or
   X¹ represents CR^{X1} and X³ represents CR^{X3};
wherein R^{X1} represents hydrogen, or methoxy; R^{X3} represents hydrogen; R⁴ represents hydrogen, or (C₁₋₄)alkyl (especially methyl); R³¹ represents hydrogen, or fluoro; and L represents a direct bond, —CHR^{L}—O—*, —CH₂—NH—*, —CH₂—N(CH₃)—*, or —O— (especially —CHR^{L}—O—*, —CH₂—NH—*, —CH₂—N(CH₃)—*, or —O—); wherein R^{L} represents hydrogen, methyl, CH₃—O—CH₂—, or (CH₃)₂NCH₂—; wherein the asterisks indicate the bond which is linked to the aromatic carbon atom;

and to the salts (in particular pharmaceutically acceptable salts) of such compounds.

16) Another embodiment relates to compounds according to any one of embodiments 1) to 9), wherein the fragment

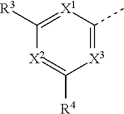

represents:

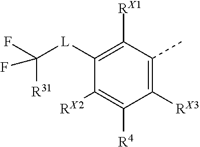

wherein R^{X1} represents hydrogen, fluoro, chloro, methyl, or methoxy; R^{X2} represents hydrogen; R^{X3} represents hydrogen, fluoro, or chloro; R⁴ represents hydrogen, fluoro, chloro, or methyl; R³¹ represents hydrogen, or fluoro; and L represents a direct bond, —CHR^{L}—O—*, —CH₂—NH—*, —CH₂—N(CH₃)—*, —O—, or; wherein R^{L} represents hydrogen, or methyl; wherein the asterisks indicate the bond which is linked to the aromatic carbon atom; or

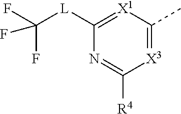

wherein
   X³ represents nitrogen and X¹ represents CR^{X1};
   X¹ represents nitrogen and X³ represents CR^{X3}; or
   X¹ represents CR^{X1} and X³ represents CR^{X3};
wherein R^{X1} represents hydrogen, or methoxy (especially hydrogen); R^{X3} represents hydrogen; R⁴ represents hydrogen, or methyl (especially hydrogen); and L represents —CHR^{L}—O—*, —CH₂—NH—*, or —CH₂—N(CH₃)—*; wherein R^{L} represents hydrogen, methyl, CH₃—O—CH₂—, or (CH₃)₂NCH₂—; wherein the asterisks indicate the bond which is linked to the aromatic carbon atom;

and to the salts (in particular pharmaceutically acceptable salts) of such compounds.

17) Another embodiment relates to compounds according to any one of embodiments 14) to 16), wherein the fragment

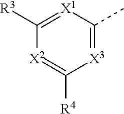

represents:

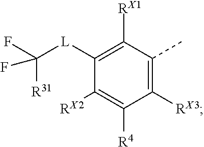

and to the salts (in particular pharmaceutically acceptable salts) of such compounds.

18) Another embodiment relates to compounds according to any one of embodiments 14) to 16), wherein the fragment

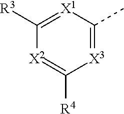

represents:

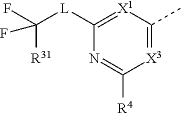

wherein
X³ represents nitrogen and X¹ represents CR^{X1};
X¹ represents nitrogen and X³ represents CR^{X3}; or
X¹ represents CR^{X1} and X³ represents CR^{X3};

and to the salts (in particular pharmaceutically acceptable salts) of such compounds.

19) Another embodiment relates to compounds according to embodiment 18), wherein X³ represents nitrogen and X¹ represents CR^{X1}; and to the salts (in particular pharmaceutically acceptable salts) of such compounds

20) Another embodiment relates to compounds according to any one of embodiments 1) to 9), wherein the fragment:

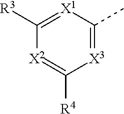

represents a ring independently selected from the following groups A) to E):

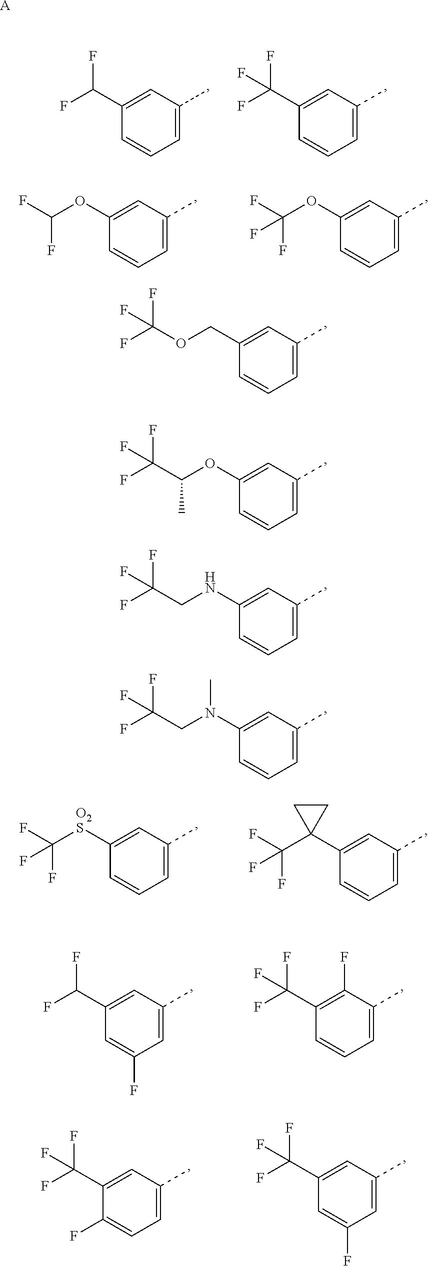

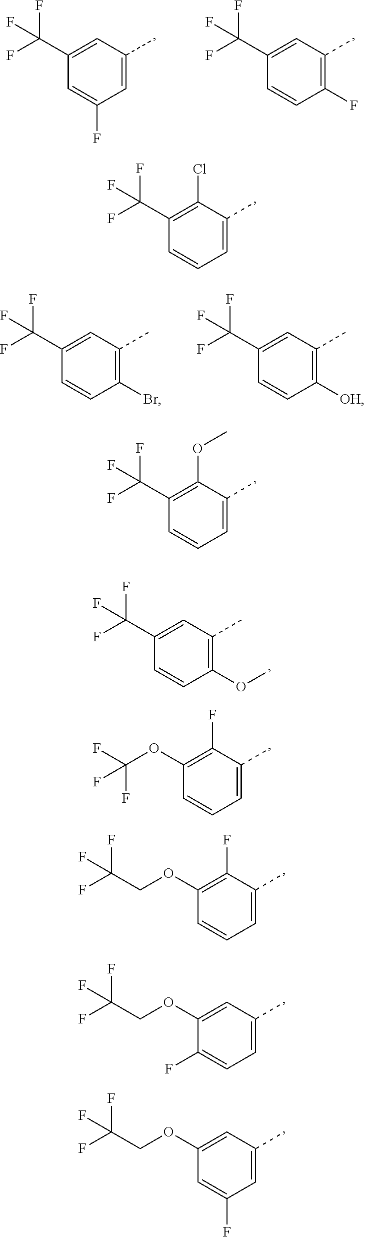

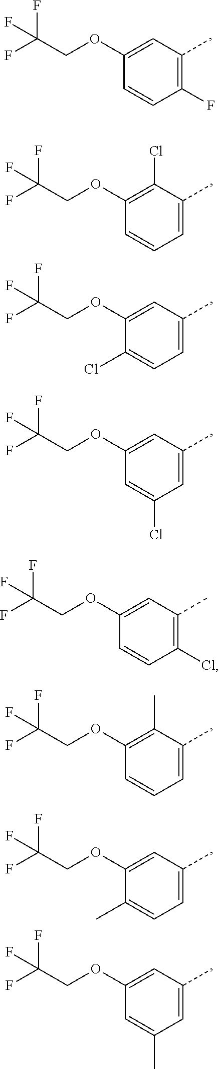

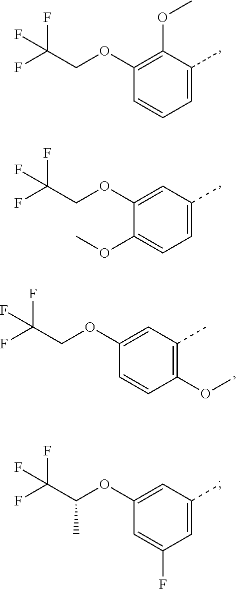

especially

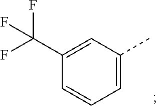

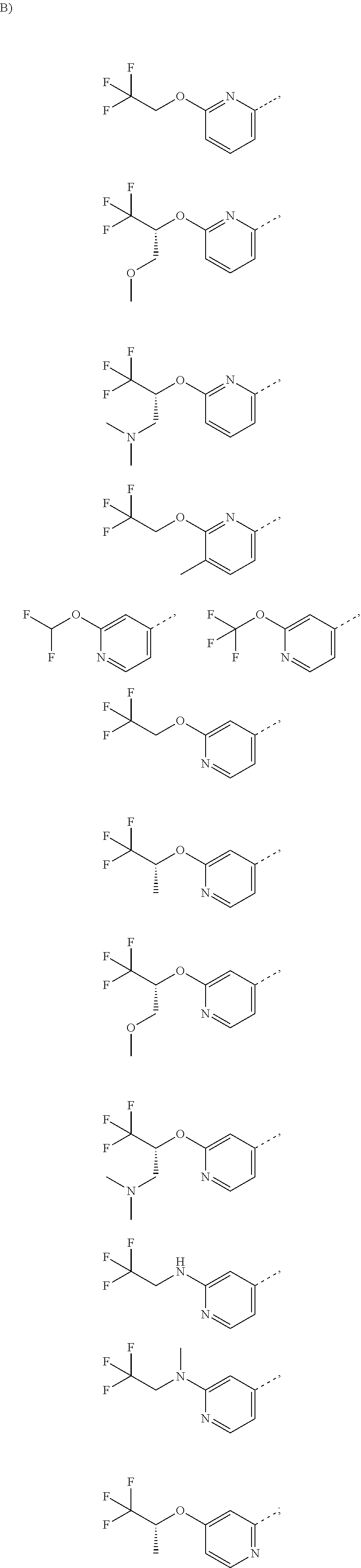

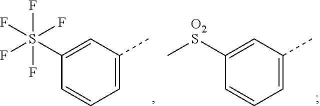

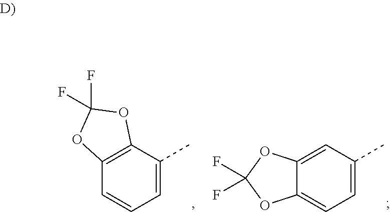

or

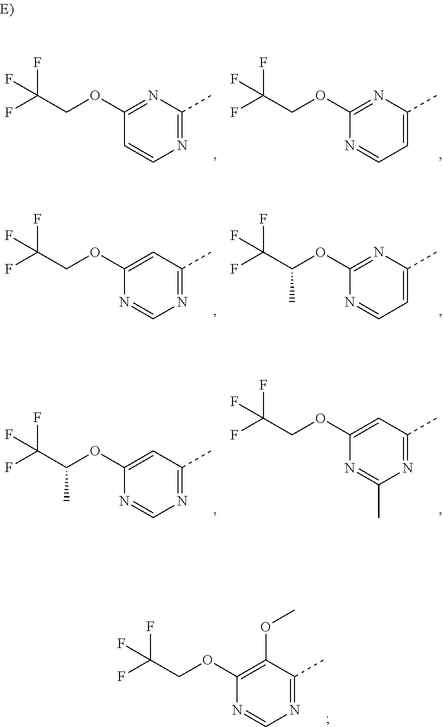

especially

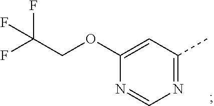

wherein each of the above groups A) and E) form a particular sub-embodiment;

and to the salts (in particular pharmaceutically acceptable salts) of such compounds.

21) Another embodiment of the invention relates to compounds of Formula (I) according to any one of embodiments 1) to 20), wherein, in case R^{2B} represents hydrogen and R^{2A} is different from hydrogen, the carbon atom to which said substituents R^{2A} and R^{2B} are attached is (R)-configurated; and to the salts (in particular pharmaceutically acceptable salts) of such compounds.

22) Another embodiment of the invention relates to compounds of Formula (I) according to any one of embodiments 1) to 20), wherein in case R^{2B} represents hydrogen and R^{2A} is different from hydrogen, the carbon atom to which said substituents R^{2A} and R^{2B} are attached is (S)-configurated; and to the salts (in particular pharmaceutically acceptable salts) of such compounds.

23) Another embodiment of the invention relates to compounds of Formula (I) according to any one of embodiments 1) to 10) and 12) to 22), wherein in case L represents —CHR^{L}—O— and R^{L} is different from hydrogen, the carbon atom to which said substituent R^{L} is attached is (R)-configurated; and to the salts (in particular pharmaceutically acceptable salts) of such compounds.

24) Another embodiment of the invention relates to compounds of Formula (I) according to any one of embodiments 1) to 10) and 12) to 22), wherein in case L represents —CHR^{L}—O— and R^{L} is different from hydrogen, the carbon atom to which said substituent R^{L} is attached is (S)-configurated; and to the salts (in particular pharmaceutically acceptable salts) of such compounds.

25) Another embodiment of the invention relates to compounds of Formula (I) according to embodiment 1), wherein

X¹ represents CR^{X1}; wherein R^{X1} represents hydrogen;

X² represents nitrogen or CR^{X2}; wherein R^{X2} represents hydrogen;

X³ represents nitrogen or CR^{X3}; wherein R^{X3} represents hydrogen;

R¹ represents hydrogen;

Y represents —CH₂—, or —CH₂—CH₂—;

R^{2A} represents hydrogen, or hydroxymethyl;

R^{2B} represents hydrogen;

R³ represents trifluoromethyl or 2,2,2-trifluoroethoxy; and

R⁴ represents hydrogen;

and to the salts (in particular pharmaceutically acceptable salts) of such compounds.

26) Another embodiment of the invention relates to compounds of Formula (I) according to embodiment 25), wherein

X² represents CR^{X2}; wherein R^{X2} represents hydrogen; and

X³ represents CR^{X3}; wherein R^{X3} represents hydrogen;

and to the salts (in particular pharmaceutically acceptable salts) of such compounds.

27) Another embodiment of the invention relates to compounds of Formula (I) according to embodiment 25), wherein X² and X³ represent nitrogen; and to the salts (in particular pharmaceutically acceptable salts) of such compounds.

28) Another embodiment of the invention relates to compounds of Formula (I) according to any one of embodiments 25) to 27), wherein Y represents —CH₂—; and to the salts (in particular pharmaceutically acceptable salts) of such compounds.

29) Another embodiment of the invention relates to compounds of Formula (I) according to any one of embodiments 25) to 27), wherein Y represents —CH₂—CH₂—; and to the salts (in particular pharmaceutically acceptable salts) of such compounds.

30) Another embodiment of the invention relates to compounds of Formula (I) according to any one of embodiments 25) to 29), wherein R^{2A} represents hydrogen; and to the salts (in particular pharmaceutically acceptable salts) of such compounds.

31) Another embodiment of the invention relates to compounds of Formula (I) according to any one of embodiments 25) to 29), wherein R^{2A} represents hydroxymethyl; and to the salts (in particular pharmaceutically acceptable salts) of such compounds.

32) A proffered embodiment of the invention relates to compounds of Formula (I) according to embodiment 31), wherein the carbon atom to which substituent R^{2A} is attached is (R)-configurated; and to the salts (in particular pharmaceutically acceptable salts) of such compounds.

33) Another embodiment of the invention relates to compounds of Formula (I) according to embodiment 31), wherein the carbon atom to which substituent R^{2A} is attached is (S)-configurated; and to the salts (in particular pharmaceutically acceptable salts) of such compounds.

34) Another embodiment of the invention relates to compounds of Formula (I) according to any one of embodiments 25) to 33), wherein R³ represents trifluoromethyl; and to the salts (in particular pharmaceutically acceptable salts) of such compounds.

35) Another embodiment of the invention relates to compounds of Formula (I) according to any one of embodiments 25) to 33), wherein R³ represents 2,2,2-trifluoroethoxy; and to the salts (in particular pharmaceutically acceptable salts) of such compounds.

36) The invention, thus, relates to compounds of the Formula (I) as defined in embodiment 1), and to such compounds further limited by the characteristics of any one of embodiments 2) to 35), under consideration of their respective dependencies; to pharmaceutically acceptable salts thereof; and to the use of such compounds as further described below. For avoidance of doubt, especially the following embodiments relating to the compounds of Formula (I) are thus possible and intended and herewith specifically disclosed in individualized form:

1, 2+1, 3+1, 3+2+1, 4+1, 4+2+1, 5+1, 5+2+1, 5+3+1, 5+3+2+1, 5+4+1, 5+4+2+1, 7+1, 7+2+1, 7+3+1, 7+3+2+1, 7+4+1, 7+4+2+1, 7+5+1, 7+5+2+1, 7+5+3+1, 7+5+3+2+1, 7+5+4+1, 7+5+4+2+1, 9+1, 9+2+1, 9+3+1, 9+3+2+1, 9+4+1, 9+4+2+1, 9+5+1, 9+5+2+1, 9+5+3+1, 9+5+3+2+1, 9+5+4+1, 9+5+4+2+1, 10+1, 10+2+1, 10+3+1, 10+3+2+1, 10+4+1, 10+4+2+1, 10+5+1, 10+5+2+1, 10+5+3+1, 10+5+3+2+1, 10+5+4+1, 10+5+4+2+1, 10+7+1, 10+7+2+1, 10+7+3+1, 10+7+3+2+1, 10+7+4+1, 10+7+4+2+1, 10+7+5+1, 10+7+5+2+1, 10+7+5+3+1, 10+7+5+3+2+1, 10+7+5+4+1, 10+7+5+4+2+1, 10+9+1, 10+9+2+1, 10+9+3+1, 10+9+3+2+1, 10+9+4+1, 10+9+4+2+1, 10+9+5+1, 10+9+5+2+1, 10+9+5+3+1, 10+9+5+3+2+1, 10+9+5+4+1, 10+9+5+4+2+1, 12+1, 12+2+1, 12+3+1, 12+3+2+1, 12+4+1, 12+4+2+1, 12+5+1, 12+5+2+1, 12+5+3+1, 12+5+3+2+1, 12+5+4+1, 12+5+4+2+1, 12+7+1, 12+7+2+1, 12+7+3+1, 12+7+3+2+1, 12+7+4+1, 12+7+4+2+1, 12+7+5+1, 12+7+5+2+1, 12+7+5+3+1, 12+7+5+3+2+1, 12+7+5+4+1, 12+7+5+4+2+1, 12+9+1, 12+9+2+1, 12+9+3+1, 12+9+3+2+1, 12+9+4+1, 12+9+4+2+1, 12+9+5+1, 12+9+5+2+1, 12+9+5+3+1, 12+9+5+3+2+1, 12+9+5+4+1, 12+9+5+4+2+1, 12+10+1, 12+10+2+1, 12+10+3+1, 12+10+3+2+1, 12+10+4+1, 12+10+4+2+1, 12+10+5+1, 12+10+5+2+1, 12+10+5+3+1, 12+10+5+3+2+1, 12+10+5+4+1, 12+10+5+4+2+1, 12+10+7+1, 12+10+7+2+1, 12+10+7+3+1, 12+10+7+3+2+1, 12+10+7+4+1, 12+10+7+4+2+1, 12+10+7+5+1, 12+10+7+5+2+1, 12+10+7+5+3+1, 12+10+7+5+3+2+1, 12+10+7+5+4+1, 12+10+7+5+4+2+1, 12+10+9+1, 12+10+9+2+1, 12+10+9+3+1, 12+10+9+3+2+1, 12+10+9+4+1, 12+10+9+4+2+1, 12+10+9+5+1, 12+10+9+5+2+1, 12+10+9+5+3+1, 12+10+9+5+3+2+1, 12+10+9+5+4+1, 12+10+9+5+4+2+1, 13+1, 13+2+1, 13+3+1, 13+3+2+1, 13+4+1, 13+4+2+1, 13+5+1, 13+5+2+1, 13+5+3+1, 13+5+3+2+1, 13+5+4+1, 13+5+4+2+1, 13+7+1, 13+7+2+1, 13+7+3+1, 13+7+3+2+1, 13+7+4+1, 13+7+4+2+1, 13+7+5+1, 13+7+5+2+1, 13+7+5+3+1, 13+7+5+3+2+1, 13+7+5+4+1, 13+7+5+4+2+1, 13+9+1, 13+9+2+1, 13+9+3+1, 13+9+3+2+1, 13+9+4+1, 13+9+4+2+1, 13+9+5+1, 13+9+5+2+1, 13+9+5+3+1, 13+9+5+3+2+1, 13+9+5+4+1, 13+9+5+4+2+1, 13+10+1, 13+10+2+1, 13+10+3+1, 13+10+3+2+1, 13+10+4+1, 13+10+4+2+1, 13+10+5+1, 13+10+5+2+1, 13+10+5+3+1, 13+10+5+3+2+1, 13+10+5+4+1, 13+10+5+4+2+1, 13+10+7+1, 13+10+7+2+1, 13+10+7+3+1, 13+10+7+3+2+1, 13+10+7+4+1, 13+10+7+4+2+1, 13+10+7+5+1, 13+10+7+5+2+1, 13+10+7+5+3+1, 13+10+7+5+3+2+1, 13+10+7+5+4+1, 13+10+7+5+4+2+1, 13+10+9+1, 13+10+9+2+1, 13+10+9+3+1, 13+10+9+3+2+1, 13+10+9+4+1, 13+10+9+4+2+1, 13+10+9+5+1, 13+10+9+5+2+1, 13+10+9+5+3+1, 13+10+9+5+3+2+1, 13+10+9+5+4+1, 13+10+9+5+4+2+1, 13+12+1, 13+12+2+1, 13+12+3+1, 13+12+3+2+1, 13+12+4+1, 13+12+4+2+1, 13+12+5+1, 13+12+5+2+1, 13+12+5+3+1, 13+12+5+3+2+1, 13+12+5+4+1, 13+12+5+4+2+1, 13+12+7+1, 13+12+7+2+1, 13+12+7+3+1, 13+12+7+3+2+1, 13+12+7+4+1, 13+12+7+4+2+1, 13+12+7+5+1, 13+12+7+5+2+1, 13+12+7+5+3+1, 13+12+7+5+3+2+1, 13+12+7+5+4+1, 13+12+7+5+4+2+1, 13+12+9+1, 13+12+9+2+1, 13+12+9+3+1, 13+12+9+3+2+1, 13+12+9+4+1, 13+12+9+4+2+1, 13+12+9+5+1, 13+12+9+5+2+1, 13+12+9+5+3+1, 13+12+9+5+3+2+1, 13+12+9+5+4+1, 13+12+9+5+4+2+1, 13+12+10+1, 13+12+10+2+1, 13+12+10+3+1, 13+12+10+3+2+1, 13+12+10+4+1, 13+12+10+4+2+1, 13+12+10+5+1, 13+12+10+5+2+1, 13+12+10+5+3+1, 13+12+10+5+3+2+1, 13+12+10+5+4+1, 13+12+10+5+4+2+1, 13+12+10+7+1, 13+12+10+7+2+1, 13+12+10+7+3+1, 13+12+10+7+3+2+1, 13+12+10+7+4+1, 13+12+10+7+4+2+1, 13+12+10+7+5+1, 13+12+10+7+5+2+1, 13+12+10+7+5+3+1, 13+12+10+7+5+3+2+1, 13+12+10+7+5+4+1, 13+12+10+7+5+4+2+1, 13+12+10+9+1, 13+12+10+9+2+1, 13+12+10+9+3+1, 13+12+10+9+3+2+1, 13+12+10+9+4+1, 13+12+10+9+4+2+1, 13+12+10+9+5+1, 13+12+10+9+5+2+1, 13+12+10+9+5+3+1, 13+12+10+9+5+3+2+1, 13+12+10+9+5+4+1, 13+12+10+9+5+4+2+1, 14+1, 14+2+1, 14+3+1, 14+3+2+1, 14+4+1, 14+4+2+1, 14+5+1, 14+5+2+1, 14+5+3+1, 14+5+3+2+1, 14+5+4+1, 14+5+4+2+1, 14+7+1, 14+7+2+1, 14+7+3+1, 14+7+3+2+1, 14+7+4+1, 14+7+4+2+1, 14+7+5+1, 14+7+5+2+1, 14+7+5+3+1, 14+7+5+3+2+1, 14+7+5+4+1, 14+7+5+4+2+1, 14+9+1, 14+9+2+1, 14+9+3+1, 14+9+3+2+1, 14+9+4+1, 14+9+4+2+1, 14+9+5+1, 14+9+5+2+1, 14+9+5+3+1, 14+9+5+3+2+1, 14+9+5+4+1, 14+9+5+4+2+1, 14+10+1, 14+10+2+1, 14+10+3+1, 14+10+3+2+1, 14+10+4+1, 14+10+4+2+1, 14+10+5+1, 14+10+5+2+1, 14+10+5+3+1, 14+10+5+3+2+1, 14+10+5+4+1, 14+10+5+4+2+1, 14+10+7+1, 14+10+7+2+1, 14+10+7+3+1, 14+10+7+3+2+1, 14+10+7+4+1, 14+10+7+4+2+1, 14+10+7+5+1, 14+10+7+5+2+1, 14+10+7+5+3+1, 14+10+7+5+3+2+1, 14+10+7+5+4+1, 14+10+7+5+4+2+1, 14+10+9+1, 14+10+9+2+1, 14+10+9+3+1, 14+10+9+3+2+1, 14+10+9+4+1, 14+10+9+4+2+1, 14+10+9+5+1, 14+10+9+5+2+1, 14+10+9+5+3+1, 14+10+9+5+3+2+1, 14+10+9+5+4+1, 14+10+9+5+4+2+1, 14+12+1, 14+12+2+1, 14+12+3+1, 14+12+3+2+1, 14+12+4+1, 14+12+4+2+1, 14+12+5+1, 14+12+5+2+1, 14+12+5+3+1, 14+12+5+3+2+1, 14+12+5+4+1, 14+12+5+4+2+1, 14+12+7+1, 14+12+7+2+1, 14+12+7+3+1, 14+12+7+3+2+1, 14+12+7+4+1, 14+12+7+4+2+1, 14+12+7+5+1, 14+12+7+5+2+1, 14+12+7+5+3+1, 14+12+7+5+3+2+1, 14+12+7+5+4+1, 14+12+7+5+4+2+1, 14+12+9+1, 14+12+9+2+1, 14+12+9+3+1, 14+12+9+3+2+1, 14+12+9+4+1, 14+12+9+4+2+1, 14+12+9+5+1, 14+12+9+5+2+1, 14+12+9+5+3+1, 14+12+9+5+3+2+1, 14+12+9+5+4+1, 14+12+9+5+4+2+1, 14+12+10+1, 14+12+10+2+1, 14+12+10+3+1, 14+12+10+3+2+1, 14+12+10+4+1, 14+12+10+4+2+1, 14+12+10+5+1, 14+12+10+5+2+1, 14+12+10+5+3+1, 14+12+10+5+3+2+1, 14+12+10+5+4+1, 14+12+10+5+4+2+1, 14+12+10+7+1, 14+12+10+7+2+1, 14+12+10+7+3+1, 14+12+10+7+3+2+1, 14+12+10+7+4+1, 14+12+10+7+4+2+1, 14+12+10+7+5+1, 14+12+10+7+5+2+1, 14+12+10+7+5+3+1, 14+12+10+7+5+3+2+1, 14+12+10+7+5+4+1, 14+12+10+7+5+4+2+1, 14+12+10+9+1, 14+12+10+9+2+1, 14+12+10+9+3+1, 14+12+10+9+3+2+1, 14+12+10+9+4+1, 14+12+10+9+4+2+1, 14+12+10+9+5+1, 14+12+10+9+5+2+1, 14+12+10+9+5+3+1, 14+12+10+9+5+3+2+1, 14+12+10+9+5+4+1, 14+12+10+9+5+4+2+1, 16+1, 16+2+1, 16+3+1, 16+3+2+1, 16+4+1, 16+4+2+1, 16+5+1, 16+5+2+1, 16+5+3+1, 16+5+3+2+1, 16+5+4+1, 16+5+4+2+1, 16+7+1, 16+7+2+1, 16+7+3+1, 16+7+3+2+1, 16+7+4+1, 16+7+4+2+1, 16+7+5+1, 16+7+5+2+1, 16+7+5+3+1, 16+7+5+3+2+1, 16+7+5+4+1, 16+7+5+4+2+1, 16+9+1, 16+9+2+1, 16+9+3+1, 16+9+3+2+1, 16+9+4+1, 16+9+4+2+1, 16+9+5+1, 16+9+5+2+1, 16+9+5+3+1, 16+9+5+3+2+1, 16+9+5+4+1, 16+9+5+4+2+1, 20+1, 20+2+1, 20+3+1, 20+3+2+1, 20+4+1, 20+4+2+1, 20+5+1, 20+5+2+1, 20+5+3+1, 20+5+3+2+1, 20+5+4+1, 20+5+4+2+1, 20+7+1, 20+7+2+1, 20+7+3+1, 20+7+3+2+1, 20+7+4+1, 20+7+4+2+1, 20+7+5+1, 20+7+5+2+1, 20+7+5+3+1, 20+7+5+3+2+1, 20+7+5+4+1, 20+7+5+4+2+1, 20+9+1, 20+9+2+1, 20+9+3+1, 20+9+3+2+1, 20+9+4+1, 20+9+4+2+1, 20+9+5+1, 20+9+5+2+1, 20+9+5+3+1, 20+9+5+3+2+1, 20+9+5+4+1, 20+9+5+4+2+1, 21+1, 21+2+1, 21+3+1, 21+3+2+1, 21+4+1, 21+4+2+1, 21+5+1, 21+5+2+1, 21+5+3+1, 21+5+3+2+1, 21+5+4+1, 21+5+4+2+1, 21+7+1, 21+7+2+1, 21+7+3+1, 21+7+3+2+1, 21+7+4+1, 21+7+4+2+1, 21+7+5+1, 21+7+5+2+1, 21+7+5+3+1, 21+7+5+3+2+1, 21+7+5+4+1, 21+7+5+4+2+1, 21+10+1, 21+10+2+1, 21+10+3+1, 21+10+3+2+1, 21+10+4+1, 21+10+4+2+1, 21+10+5+1, 21+10+5+2+1, 21+10+5+3+1, 21+10+5+3+2+1, 21+10+5+4+1, 21+10+5+4+2+1, 21+10+7+1, 21+10+7+2+1, 21+10+7+3+1, 21+10+7+3+2+1, 21+10+7+4+1, 21+10+7+4+2+1, 21+10+7+5+1, 21+10+7+5+2+1, 21+10+7+5+3+1, 21+10+7+5+3+2+1, 21+10+7+5+4+1, 21+10+7+5+4+2+1, 21+10+9+1, 21+10+9+2+1, 21+10+9+3+1, 21+10+9+3+2+1, 21+10+9+4+1, 21+10+9+4+2+1, 21+10+9+5+1, 21+10+9+5+2+1, 21+10+9+5+3+1, 21+10+9+5+3+2+1, 21+10+9+5+4+1, 21+10+9+5+4+2+1, 21+20+1, 21+20+2+1, 21+20+3+1, 21+20+3+2+1, 21+20+4+1, 21+20+4+2+1, 21+20+5+1, 21+20+5+2+1, 21+20+5+3+1, 21+20+5+3+2+1, 21+20+5+4+1, 21+20+5+4+2+1, 21+20+7+1, 21+20+7+2+1, 21+20+7+3+1, 21+20+7+3+2+1, 21+20+7+4+1, 21+20+7+4+2+1, 21+20+7+5+1, 21+20+7+5+2+1, 21+20+7+5+3+1, 21+20+7+5+3+2+1, 21+20+7+5+4+1, 21+20+7+5+4+2+1, 21+20+9+1, 21+20+9+2+1, 21+20+9+3+1, 21+20+9+3+2+1, 21+20+9+4+1, 21+20+9+4+2+1, 21+20+9+5+1, 21+20+9+5+2+1, 21+20+9+5+3+1, 21+20+9+5+3+2+1, 21+20+9+5+4+1, 21+20+9+5+4+2+1, 22+1, 22+2+1, 22+3+1, 22+3+2+1, 22+4+1, 22+4+2+1, 22+5+1, 22+5+2+1, 22+5+3+1, 22+5+3+2+1, 22+5+4+1, 22+5+4+2+1, 22+7+1, 22+7+2+1, 22+7+3+1, 22+7+3+2+1, 22+7+4+1, 22+7+4+2+1, 22+7+5+1, 22+7+5+2+1, 22+7+5+3+1, 22+7+5+3+2+1, 22+7+5+4+1, 22+7+5+4+2+1, 22+10+1, 22+10+2+1, 22+10+3+1, 22+10+3+2+1, 22+10+4+1, 22+10+4+2+1, 22+10+5+1, 22+10+5+2+1, 22+10+5+3+1, 22+10+5+3+2+1, 22+10+5+4+1, 22+10+5+4+2+1, 22+10+7+1, 22+10+7+2+1, 22+10+7+3+1, 22+10+7+3+2+1, 22+10+7+4+1, 22+10+7+4+2+1, 22+10+7+5+1, 22+10+7+5+2+1, 22+10+7+5+3+1, 22+10+7+5+3+2+1, 22+10+7+5+4+1, 22+10+7+5+4+2+1, 22+10+9+1, 22+10+9+2+1, 22+10+9+3+1, 22+10+9+3+2+1, 22+10+9+4+1, 22+10+9+4+2+1, 22+10+9+5+1, 22+10+9+5+2+1, 22+10+9+5+3+1, 22+10+9+5+3+2+1, 22+10+9+5+4+1, 22+10+9+5+4+2+1, 22+20+1, 22+20+2+1, 22+20+3+1, 22+20+3+2+1, 22+20+4+1, 22+20+4+2+1, 22+20+5+1, 22+20+5+2+1, 22+20+5+3+1, 22+20+5+3+2+1, 22+20+5+4+1, 22+20+5+4+2+1, 22+20+7+1, 22+20+7+2+1, 22+20+7+3+1, 22+20+7+3+2+1, 22+20+7+4+1, 22+20+7+4+2+1, 22+20+7+5+1, 22+20+7+5+2+1, 22+20+7+5+3+1, 22+20+7+5+3+2+1, 22+20+7+5+4+1, 22+20+7+5+4+2+1, 22+20+9+1, 22+20+9+2+1, 22+20+9+3+1, 22+20+9+3+2+1, 22+20+9+4+1, 22+20+9+4+2+1, 22+20+9+5+1, 22+20+9+5+2+1, 22+20+9+5+3+1, 22+20+9+5+3+2+1, 22+20+9+5+4+1, 22+20+9+5+4+2+1, 23+1, 23+2+1, 23+3+1, 23+3+2+1, 23+4+1, 23+4+2+1, 23+5+1, 23+5+2+1, 23+5+3+1, 23+5+3+2+1, 23+5+4+1, 23+5+4+2+1, 23+7+1, 23+7+2+1, 23+7+3+1, 23+7+3+2+1, 23+7+4+1, 23+7+4+2+1, 23+7+5+1, 23+7+5+2+1, 23+7+5+3+1, 23+7+5+3+2+1, 23+7+5+4+1, 23+7+5+4+2+1, 23+10+1, 23+10+2+1, 23+10+3+1, 23+10+3+2+1, 23+10+4+1, 23+10+4+2+1, 23+10+5+1, 23+10+5+2+1, 23+10+5+3+1, 23+10+5+3+2+1, 23+10+5+4+1, 23+10+5+4+2+1, 23+10+7+1, 23+10+7+2+1, 23+10+7+3+1, 23+10+7+3+2+1, 23+10+7+4+1, 23+10+7+4+2+1, 23+10+7+5+1, 23+10+7+5+2+1, 23+10+7+5+3+1, 23+10+7+5+3+2+1, 23+10+7+5+4+1, 23+10+7+5+4+2+1, 23+10+9+1, 23+10+9+2+1, 23+10+9+3+1, 23+10+9+3+2+1, 23+10+9+4+1, 23+10+9+4+2+1, 23+10+9+5+1, 23+10+9+5+2+1, 23+10+9+5+3+1, 23+10+9+5+3+2+1, 23+10+9+5+4+1, 23+10+9+5+4+2+1, 23+20+1, 23+20+2+1, 23+20+3+1, 23+20+3+2+1, 23+20+4+1, 23+20+4+2+1, 23+20+5+1, 23+20+5+2+1, 23+20+5+3+1, 23+20+5+3+2+1, 23+20+5+4+1, 23+20+5+4+2+1, 23+20+7+1, 23+20+7+2+1, 23+20+7+3+1, 23+20+7+3+2+1, 23+20+7+4+1, 23+20+7+4+2+1, 23+20+7+5+1, 23+20+7+5+2+1, 23+20+7+5+3+1, 23+20+7+5+3+2+1, 23+20+7+5+4+1, 23+20+7+5+4+2+1, 23+20+9+1, 23+20+9+2+1, 23+20+9+3+1, 23+20+9+3+2+1, 23+20+9+4+1, 23+20+9+4+2+1, 23+20+9+5+1, 23+20+9+5+2+1, 23+20+9+5+3+1, 23+20+9+5+3+2+1, 23+20+9+5+4+1, 23+20+9+5+4+2+1, 24+1, 24+2+1, 24+3+1, 24+3+2+1, 24+4+1, 24+4+2+1, 24+5+1, 24+5+2+1, 24+5+3+1, 24+5+3+2+1, 24+5+4+1, 24+5+4+2+1, 24+7+1, 24+7+2+1, 24+7+3+1, 24+7+3+2+1, 24+7+4+1, 24+7+4+2+1, 24+7+5+1, 24+7+5+2+1, 24+7+5+3+1, 24+7+5+3+2+1, 24+7+5+4+1, 24+7+5+4+2+1, 24+10+1, 24+10+2+1, 24+10+3+1, 24+10+3+2+1, 24+10+4+1, 24+10+4+2+1, 24+10+5+1, 24+10+5+2+1, 24+10+5+3+1, 24+10+5+3+2+1, 24+10+5+4+1, 24+10+5+4+2+1, 24+10+7+1, 24+10+7+2+1, 24+10+7+3+1, 24+10+7+3+2+1, 24+10+7+4+1, 24+10+7+4+2+1, 24+10+7+5+1, 24+10+7+5+2+1, 24+10+7+5+3+1, 24+10+7+5+3+2+1, 24+10+7+5+4+1, 24+10+7+5+4+2+1, 24+10+9+1, 24+10+9+2+1, 24+10+9+3+1, 24+10+9+3+2+1, 24+10+9+4+1, 24+10+9+4+2+1, 24+10+9+5+1, 24+10+9+5+2+1, 24+10+9+5+3+1, 24+10+9+5+3+2+1, 24+10+9+5+4+1, 24+10+9+5+4+2+1, 24+20+1, 24+20+2+1, 24+20+3+1, 24+20+3+2+1, 24+20+4+1, 24+20+4+2+1, 24+20+5+1, 24+20+5+2+1, 24+20+5+3+1, 24+20+5+3+2+1, 24+20+5+4+1, 24+20+5+4+2+1, 24+20+7+1, 24+20+7+2+1, 24+20+7+3+1, 24+20+7+3+2+1, 24+20+7+4+1, 24+20+7+4+2+1, 24+20+7+5+1, 24+20+7+5+2+1, 24+20+7+5+3+1, 24+20+7+5+3+2+1, 24+20+7+5+4+1, 24+20+7+5+4+2+1, 24+20+9+1, 24+20+9+2+1, 24+20+9+3+1, 24+20+9+3+2+1, 24+20+9+4+1, 24+20+9+4+2+1, 24+20+9+5+1, 24+20+9+5+2+1, 24+20+9+5+3+1, 24+20+9+5+3+2+1, 24+20+9+5+4+1, 24+20+9+5+4+2+1, 25+1, 26+25+1, 27+25+1, 28+25+1, 28+26+25+1, 28+27+25+1, 29+25+1, 29+26+25+1, 29+27+25+1, 30+25+1, 30+26+25+1, 30+27+25+1, 30+28+25+1, 30+28+26+25+1, 30+28+27+25+1, 30+29+25+1, 30+29+26+25+1, 30+29+27+25+1, 31+25+1, 31+26+25+1, 31+27+25+1, 31+28+25+1, 31+28+26+25+1, 31+28+27+25+1, 31+29+25+1, 31+29+26+25+1, 31+29+27+25+1, 32+31+25+1, 32+31+26+25+1, 32+31+27+25+1, 32+31+28+25+1, 32+31+28+26+25+1, 32+31+28+27+25+1, 32+31+29+25+1, 32+31+29+26+25+1, 32+31+29+27+25+1, 33+31+25+1, 33+31+26+25+1, 33+31+27+25+1, 33+31+28+25+1, 33+31+28+26+25+1, 33+31+28+27+25+1, 33+31+29+25+1, 33+31+29+26+25+1, 33+31+29+27+25+1, 34+25+1, 34+26+25+1, 34+27+25+1, 34+28+25+1, 34+28+26+25+1, 34+28+27+25+1, 34+29+25+1, 34+29+26+25+1, 34+29+27+25+1, 34+30+25+1, 34+30+26+25+1, 34+30+27+25+1, 34+30+28+25+1, 34+30+28+26+25+1, 34+30+28+27+25+1, 34+30+29+25+1, 34+30+29+26+25+1, 34+30+29+27+25+1, 34+31+25+1, 34+31+26+25+1, 34+31+27+25+1, 34+31+28+25+1, 34+31+28+26+25+1, 34+31+28+27+25+1, 34+31+29+25+1, 34+31+29+26+25+1, 34+31+29+27+25+1, 34+32+31+25+1, 34+32+31+26+25+1, 34+32+31+27+25+1, 34+32+31+28+25+1, 34+32+31+28+26+25+1, 34+32+31+28+27+25+1, 34+32+31+29+25+1, 34+32+31+29+26+25+1, 34+32+31+29+27+25+1, 34+33+31+25+1, 34+33+31+26+25+1, 34+33+31+27+25+1, 34+33+31+28+25+1, 34+33+31+28+26+25+1, 34+33+31+28+27+25+1, 34+33+31+29+25+1, 34+33+31+29+26+25+1, 34+33+31+29+27+25+1, 35+25+1, 35+26+25+1, 35+27+25+1, 35+28+25+1, 35+28+26+25+1, 35+28+27+25+1, 35+29+25+1, 35+29+26+25+1, 35+29+27+25+1, 35+30+25+1, 35+30+26+25+1, 35+30+27+25+1, 35+30+28+25+1, 35+30+28+26+25+1, 35+30+28+27+25+1, 35+30+29+25+1, 35+30+29+26+25+1, 35+30+29+27+25+1, 35+31+25+1, 35+31+26+25+1, 35+31+27+25+1, 35+31+28+25+1, 35+31+28+26+25+1, 35+31+28+27+25+1, 35+31+29+25+1, 35+31+29+26+25+1, 35+31+29+27+25+1, 35+32+31+25+1, 35+32+31+26+25+1, 35+32+31+27+25+1, 35+32+31+28+25+1, 35+32+31+28+26+25+1, 35+32+31+28+27+25+1, 35+32+31+29+25+1, 35+32+31+29+26+25+1, 35+32+31+29+27+25+1, 35+33+31+25+1, 35+33+31+26+25+1, 35+33+31+27+25+1, 35+33+31+28+25+1, 35+33+31+28+26+25+1, 35+33+31+28+27+25+1, 35+33+31+29+25+1, 35+33+31+29+26+25+1, 35+33+31+29+27+25+1.

In the list above the numbers refer to the embodiments according to their numbering provided hereinabove whereas “+” indicates the dependency from another embodiment. The different individualized embodiments are separated by commas. In other words, “20+9+4+1” for example refers to embodiment 20) depending on embodiment 9), depending on embodiment 4), depending on embodiment 1), i.e. embodiment “20+9+4+1” corresponds to the compounds of embodiment 1) further limited by the features of the embodiments 4), 9) and 20).

37) Another embodiment relates to compounds of Formula (I) according to embodiment 1), which are selected from the following compounds:
1-Spiro[3.3]hept-2-yl-3-(3-trifluoromethyl-benzyl)-urea;
1-Spiro[2.3]hex-5-yl-3-(3-trifluoromethyl-benzyl)-urea;
1-(1,1-Difluoro-spiro[2.3]hex-5-yl)-3-(3-trifluoromethyl-benzyl)-urea;
1-((3r,5r)-1,1-Difluoro-spiro[2.3]hex-54)-3-(3-trifluoromethyl-benzyl)-urea;
1-((3s,5s)-1,1-Difluoro-spiro[2.3]hex-5-yl)-3-(3-trifluoromethyl-benzyl)-urea;
1-(6,6-Difluoro-spiro[3.3]hept-2-yl)-3-(3-trifluoromethyl-benzyl)-urea;
1-[1-(3-Difluoromethoxy-phenyl)-cyclopropyl]-3-spiro[3.3]hept-2-yl-urea;
1-(2-Fluoro-3-trifluoromethoxy-benzyl)-3-spiro[3.3]hept-2-yl-urea;
1-[2-Methoxy-1-(3-trifluoromethyl-phenyl)-ethyl]-3-spiro[3.3]hept-2-yl-urea;
1-[1-(2-Difluoromethoxy-pyridin-4-yl)-2-methoxy-ethyl]-3-spiro[3.3]hept-2-yl-urea;
1-Spiro[3.3]hept-2-yl-3-(5-trifluoromethoxy-2,3-dihydro-benzofuran-3-yl)-urea;
1-(2-Fluoro-3-trifluoromethyl-benzyl)-3-spiro[3.3]hept-2-yl-urea;
1-(3-Difluoromethoxy-benzyl)-3-spiro[3.3]hept-2-yl-urea;
1-Spiro[3.3]hept-2-yl-3-[3-(2,2,2-trifluoro-ethoxy)-benzyl]-urea;
1-(3-Difluoromethyl-benzyl)-3-spiro[3.3]hept-2-yl-urea;
1-(2-Fluoro-5-trifluoromethyl-benzyl)-3-spiro[3.3]hept-2-yl-urea;
1-(4-Fluoro-3-trifluoromethyl-benzyl)-3-spiro[3.3]hept-2-yl-urea;
1-(3-Fluoro-5-trifluoromethyl-benzyl)-3-spiro[3.3]hept-2-yl-urea;
1-[1-(2-Bromo-5-trifluoromethyl-phenyl)-ethyl]-3-spiro[3.3]hept-2-yl-urea;
1-[1-(2-Methoxy-5-trifluoromethyl-phenyl)-ethyl]-3-spiro[3.3]hept-2-yl-urea;
1-[1-(2-Methoxy-3-trifluoromethyl-phenyl)-ethyl]-3-spiro[3.3]hept-2-yl-urea;
1-(2-Methoxy-3-trifluoromethyl-benzyl)-3-spiro[3.3]hept-2-yl-urea;
1-Spiro[3.3]hept-2-yl-3-[1-(3-trifluoromethyl-phenyl)-ethyl]-urea;
1-[Cyclopropyl-(3-trifluoromethyl-phenyl)-methyl]-3-spiro[3.3]hept-2-yl-urea;
1-[Cyclopropyl-(3-difluoromethoxy-phenyl)-methyl]-3-spiro[3.3]hept-2-yl-urea;
1-{1-[4-Methyl-3-(2,2,2-trifluoro-ethoxy)-phenyl]-ethyl}-3-spiro[3.3]hept-2-yl-urea;
1-{1-[4-Chloro-3-(2,2,2-trifluoro-ethoxy)-phenyl]-ethyl}-3-spiro[3.3]hept-2-yl-urea;
1-{1-[4-Methoxy-3-(2,2,2-trifluoro-ethoxy)-phenyl]-ethyl}-3-spiro[3.3]hept-2-yl-urea;
1-{1-[2-Methyl-3-(2,2,2-trifluoro-ethoxy)-phenyl]-ethyl}-3-spiro[3.3]hept-2-yl-urea;
1-{1-[2-Chloro-3-(2,2,2-trifluoro-ethoxy)-phenyl]-ethyl}-3-spiro[3.3]hept-2-yl-urea;
1-{1-[2-Methoxy-3-(2,2,2-trifluoro-ethoxy)-phenyl]-ethyl}-3-spiro[3.3]hept-2-yl-urea;
1-{1-[3-Methyl-5-(2,2,2-trifluoro-ethoxy)-phenyl]-ethyl}-3-spiro[3.3]hept-2-yl-urea;
1-{1-[3-Chloro-5-(2,2,2-trifluoro-ethoxy)-phenyl]-ethyl}-3-spiro[3.3]hept-2-yl-urea;
1-(1-(3-(pentafluoro-16-sulfaneyl)phenyl)ethyl)-3-(spiro[3.3]heptan-2-yl)urea;
1-(3-(pentafluoro-16-sulfaneyl)benzyl)-3-(spiro[3.3]heptan-2-yl)urea;
1-[1-(3-Methanesulfonyl-phenyl)-ethyl]-3-spiro[3.3]hept-2-yl-urea;
1-Spiro[3.3]hept-2-yl-3-[3-(1-trifluoromethyl-cyclopropyl)-benzyl]-urea;
1-(2-Hydroxy-5-trifluoromethyl-benzyl)-3-spiro[3.3]hept-2-yl-urea;
1-{1-[2-Chloro-5-(2,2,2-trifluoro-ethoxy)-phenyl]-ethyl}-3-spiro[3.3]hept-2-yl-urea;
1-{1-[2-Methoxy-5-(2,2,2-trifluoro-ethoxy)-phenyl]-ethyl}-3-spiro[3.3]hept-2-yl-urea;
1-Spiro[3.3]hept-2-yl-3-(3-trifluoromethoxy-benzyl)-urea;
1-Spiro[3.3]hept-2-yl-3-(3-trifluoromethanesulfonyl-benzyl)-urea;
1-Spiro[3.3]hept-2-yl-3-(3-trifluoromethoxymethyl-benzyl)-urea;
1-Spiro[3.3]hept-2-yl-3-[3-(3-trifluoromethyl-phenyl)-oxetan-3-yl]-urea;
1-[2-Hydroxy-2-methyl-1-(3-trifluoromethyl-phenyl)-propyl]-3-spiro[3.3]hept-2-yl-urea;
1-(2,2-Difluoro-benzo[1,3]dioxol-4-ylmethyl)-3-spiro[3.3]hept-2-yl-urea;
1-[1-(2,2-Difluoro-benzo[1,3]dioxol-4-yl)-ethyl]-3-spiro[3.3]hept-2-yl-urea;
1-{2-Dimethylamino-1-[6-(2,2,2-trifluoro-ethoxy)-pyridin-2-yl]-ethyl}-3-spiro[3.3]hept-2-yl-urea;
1-Spiro[3.3]hept-2-yl-3-{4-[2-(2,2,2-trifluoro-ethoxy)-pyridin-4-yl]-tetrahydro-pyran-4-yl}-urea;
1-{2-Methoxy-1-[2-(2,2,2-trifluoro-ethoxy)-pyridin-4-yl]-ethyl}-1-methyl-3-spiro[3.3]hept-2-yl-urea;
1-[1-(2-Difluoromethoxy-pyridin-4-yl)-cyclobutyl]-3-spiro[3.3]hept-2-yl-urea;
1-[1-(2-Difluoromethoxy-pyridin-4-yl)-1-methyl-ethyl]-3-spiro[3.3]hept-2-yl-urea;
5-(2,2,2-Trifluoro-ethoxy)-3,4-dihydro-1H-[2,6]naphthyridine-2-carboxylic acid spiro[3.3]hept-2-ylamide;
1-Methoxymethyl-5-(2,2,2-trifluoro-ethoxy)-3,4-dihydro-1H-isoquinoline-2-carboxylic acid spiro[3.3]hept-2-ylamide;
1-[Cyano-(3-trifluoromethyl-phenyl)-methyl]-3-spiro[3.3]hept-2-yl-urea;
1-Spiro[3.3]hept-2-yl-3-[(1H-[1,2,4]triazol-3-yl)-(3-trifluoromethyl-phenyl)-methyl]-urea;
1-[2,2-Difluoro-1-(3-trifluoromethyl-phenyl)-ethyl]-3-spiro[3.3]hept-2-yl-urea;
1-Spiro[3.3]hept-2-yl-3-[2,2,2-trifluoro-1-(3-trifluoromethyl-phenyl)-ethyl]-urea;
1-Spiro[3.3]hept-2-yl-3-[6-(2,2,2-trifluoro-ethoxy)-pyrimidin-4-ylmethyl]-urea;
1-[2-Methyl-6-(2,2,2-trifluoro-ethoxy)-pyrimidin-4-ylmethyl]-3-spiro[3.3]hept-2-yl-urea;
1-Spiro[3.3]hept-2-yl-3-{1-[6-(2,2,2-trifluoro-ethoxy)-pyrimidin-4-yl]-ethyl}-urea;
2-(3-Spiro[3.3]hept-2-yl-ureido)-2-(3-trifluoromethyl-phenyl)-acetamide;
N-Methyl-2-(3-spiro[3.3]hept-2-yl-ureido)-2-(3-trifluoromethyl-phenyl)-acetamide;
1-[2-Methanesulfonyl-1-(3-trifluoromethoxy-phenyl)-ethyl]-3-spiro[3.3]hept-2-yl-urea;
1-[1-(2,2-Difluoro-benzo[1,3]dioxol-5-yl)-ethyl]-3-spiro[3.3]hept-2-yl-urea;
1-[2-Cyano-1-(3-trifluoromethoxy-phenyl)-ethyl]-3-spiro[3.3]hept-2-yl-urea;
1-[2-Cyano-1-(3-trifluoromethyl-phenyl)-ethyl]-3-spiro[3.3]hept-2-yl-urea;
1-{1-[2-Methyl-6-(2,2,2-trifluoro-ethoxy)-pyrimidin-4-yl]-ethyl)-3-spiro[3.3]hept-2-yl-urea;
1-[2-Methanesulfonyl-1-(3-trifluoromethyl-phenyl)-ethyl]-3-spiro[3.3]hept-2-yl-urea;
1-Spiro[3.3]hept-2-yl-3-[2-(2,2,2-trifluoro-ethoxy)-pyrimidin-4-ylmethyl]-urea;
1-[2-(2-Methoxy-ethoxy)-1-(3-trifluoromethyl-phenyl)-ethyl]-3-spiro[3.3]hept-2-yl-urea;
1-[2-Isopropoxy-1-(3-trifluoromethyl-phenyl)-ethyl]-3-spiro[3.3]hept-2-yl-urea;
1-[3-Ethoxy-1-(3-trifluoromethyl-phenyl)-propyl]-3-spiro[3.3]hept-2-yl-urea;
1-[3-Hydroxy-1-(3-trifluoromethyl-phenyl)-propyl]-3-spiro[3.3]hept-2-yl-urea;
1-[3-Methylsulfanyl-1-(3-trifluoromethyl-phenyl)-propyl]-3-spiro[3.3]hept-2-yl-urea;
1-[2-Hydroxy-1-(3-trifluoromethyl-phenyl)-ethyl]-3-spiro[3.3]hept-2-yl-urea;
1-{3-Ethoxy-1-[2-(2,2,2-trifluoro-ethoxy)-pyridin-4-yl]-propyl)-3-spiro[3.3]hept-2-yl-urea;
1-{2-Methoxy-1-[6-(2,2,2-trifluoro-ethoxy)-pyrimidin-4-yl]-ethyl}-3-spiro[3.3]hept-2-yl-urea;
4-(3-Spiro[3.3]hept-2-yl-ureido)-4-(3-trifluoromethyl-phenyl)-butyramide;
1-Spiro[3.3]hept-2-yl-3-[1-(3-trifluoromethyl-phenyl)-but-3-enyl]-urea;
1-[3-Methanesulfonyl-1-(3-trifluoromethyl-phenyl)-propyl]-3-spiro[3.3]hept-2-yl-urea;
1-{2-Hydroxy-1-[2-methyl-6-(2,2,2-trifluoro-ethoxy)-pyrimidin-4-yl]-ethyl}-3-spiro[3.3]hept-2-yl-urea;
1-{2-Hydroxy-1-[6-(2,2,2-trifluoro-ethoxy)-pyrimidin-4-yl]-ethyl}-3-spiro[3.3]hept-2-yl-urea;
1-[3,3-Difluoro-1-(3-trifluoromethyl-phenyl)-propyl]-3-spiro[3.3]hept-2-yl-urea;
1-{3-Hydroxy-1-[6-(2,2,2-trifluoro-ethoxy)-pyrimidin-4-yl]-propyl}-3-spiro[3.3]hept-2-yl-urea;
1-[1-(3-Methanesulfonyl-phenyl)-2-methoxy-ethyl]-3-spiro[3.3]hept-2-yl-urea;
1-{2-Hydroxy-1-[3-(2,2,2-trifluoro-ethoxy)-phenyl]-ethyl}-3-spiro[3.3]hept-2-yl-urea;
1-{3-Hydroxy-1-[3-(2,2,2-trifluoro-ethoxy)-phenyl]-propyl)-3-spiro[3.3]hept-2-yl-urea;
1-[Cyano-(3-trifluoromethyl-phenyl)-methyl]-3-spiro[3.3]hept-2-yl-urea;
1-Spiro[3.3]hept-2-yl-3-{1-[3-(2,2,2-trifluoro-ethoxy)-phenyl]-ethyl)-urea;
1-{2-Methoxy-1-[3-(2,2,2-trifluoro-ethoxy)-phenyl]-ethyl}-3-spiro[3.3]hept-2-yl-urea;
1-{2-Methoxy-1-[2-(2,2,2-trifluoro-ethoxy)-pyridin-4-yl]-ethyl}-3-spiro[3.3]hept-2-yl-urea;
1-Spiro[3.3]hept-2-yl-3-{1-[2-(2,2,2-trifluoro-ethoxy)-pyridin-4-yl]-ethyl}-urea;
1-{2-Dimethylamino-1-[2-(2,2,2-trifluoro-ethoxy)-pyridin-4-yl]-ethyl}-3-spiro[3.3]hept-2-yl-urea;
1-{1-[3-Fluoro-5-(2,2,2-trifluoro-ethoxy)-phenyl]-ethyl}-3-spiro[3.3]hept-2-yl-urea;
1-Spiro[3.3]hept-2-yl-3-(2-trifluoromethoxy-pyridin-4-ylmethyl)-urea;
1-Spiro[3.3]hept-2-yl-3-[1-(2-trifluoromethoxy-pyridin-4-yl)-ethyl]-urea;
1-{1-[2-Fluoro-3-(2,2,2-trifluoro-ethoxy)-phenyl]-ethyl}-3-spiro[3.3]hept-2-yl-urea; 1-{1-[2-Fluoro-5-(2,2,2-trifluoro-ethoxy)- phenyl]-ethyl}-3-spiro[3.3]hept-2-yl-urea;
1-[1-(2-Difluoromethoxy-pyridin-4-yl)-ethyl]-3-spiro[3.3]hept-2-yl-urea;
1-(Spiro[3.3]heptan-2-yl)-3-(1-(2-((1,1,1-trifluoropropan-2-yl)oxy)pyridin-4-yl)ethyl)urea;
1-(Spiro[3.3]heptan-2-yl)-3-(1-(3-((1,1,1-trifluoropropan-2-yl)oxy)phenyl)ethyl)urea;
1-Spiro[3.3]hept-2-yl-3-[2-(2,2,2-trifluoro-ethoxy)-pyridin-4-ylmethyl]-urea;
1-Spiro[3.3]hept-2-yl-3-[6-(2,2,2-trifluoro-1-methoxymethyl-ethoxy)-pyridin-2-ylmethyl]-urea;
1-[6-(1-Dimethylaminomethyl-2,2,2-trifluoro-ethoxy)-pyridin-2-ylmethyl]-3-spiro[3.3]hept-2-yl-urea;
1-[2-(1-Dimethylaminomethyl-2,2,2-trifluoro-ethoxy)-pyridin-4-ylmethyl]-3-spiro[3.3]hept-2-yl-urea;
1-Spiro[3.3]hept-2-yl-3-[2-(2,2,2-trifluoro-1-methoxymethyl-ethoxy)-pyridin-4-ylmethyl]-urea;
1-[5-Methyl-6-(2,2,2-trifluoro-ethoxy)-pyridin-2-ylmethyl]-3-spiro[3.3]hept-2-yl-urea;
1-[4-Methyl-3-(2,2,2-trifluoro-ethoxy)-benzyl]-3-spiro[3.3]hept-2-yl-urea;
1-[3-Fluoro-5-(2,2,2-trifluoro-1-methyl-ethoxy)-benzyl]-3-spiro[3.3]hept-2-yl-urea;
1-Spiro[3.3]hept-2-yl-3-{1-[3-(2,2,2-trifluoro-1-methyl-ethoxy)-phenyl]-cyclopropyl}-urea;
1-Spiro[3.3]hept-2-yl-3-[4-(2,2,2-trifluoro-ethoxy)-pyrimidin-2-ylmethyl]-urea;
1-{1-[4-Fluoro-3-(2,2,2-trifluoro-ethoxy)-phenyl]-ethyl}-3-spiro[3.3]hept-2-yl-urea;
1-[4-Fluoro-3-(2,2,2-trifluoro-ethoxy)-benzyl]-3-spiro[3.3]hept-2-yl-urea;
1-{2-[Methyl-(2,2,2-trifluoro-ethyl)-amino]-pyridin-4-ylmethyl}-3-spiro[3.3]hept-2-yl-urea;
1-Spiro[3.3]hept-2-yl-3-[2-(2,2,2-trifluoro-ethylamino)-pyridin-4-ylmethyl]-urea;
1-Spiro[3.3]hept-2-yl-3-[4-(2,2,2-trifluoro-1-methyl-ethoxy)-pyridin-2-ylmethyl]-urea;
1-Spiro[3.3]hept-2-yl-3-[2-(2,2,2-trifluoro-1-methyl-ethoxy)-pyrimidin-4-ylmethyl]-urea;
1-Spiro[3.3]hept-2-yl-3-[1-(3-trifluoromethoxy-phenyl)-ethyl]-urea;
1-Spiro[2.3]hex-5-yl-3-[6-(2,2,2-trifluoro-1-methyl-ethoxy)-pyrimidin-4-ylmethyl]-urea;
1-[1-(2,2-Difluoro-benzo[1,3]dioxol-4-yl)-ethyl]-3-spiro[2.3]hex-5-yl-urea;
1-{2-Hydroxy-1-[6-(2,2,2-trifluoro-ethoxy)-pyrimidin-4-yl]-ethyl}-3-spiro[2.3]hex-5-yl-urea;
1-{2-Hydroxy-1-[2-methyl-6-(2,2,2-trifluoro-ethoxy)-pyrimidin-4-yl]-ethyl}-3-spiro[2.3]hex-5-yl-urea;
1-[1-(3-Difluoromethoxy-phenyl)-ethyl]-3-spiro[2.3]hex-5-yl-urea;
1-(2-Methoxy-5-trifluoromethyl-benzyl)-3-spiro[2.3]hex-5-yl-urea;
1-Spiro[2.3]hex-5-yl-3-[4-(2,2,2-trifluoro-ethoxy)-pyrimidin-2-ylmethyl]-urea;
1-(1-(3-(pentafluoro-16-sulfaneyl)phenyl)ethyl)-3-(spiro[2.3]hexan-5-yl)urea;
1-(2-Methoxy-3-trifluoromethyl-benzyl)-3-spiro[2.3]hex-5-yl-urea;
1-{1-[2-Fluoro-5-(2,2,2-trifluoro-ethoxy)-phenyl]-ethyl}-3-spiro[2.3]hex-5-yl-urea;
1-(3-(pentafluoro-16-sulfaneyl)benzyl)-3-(spiro[2.3]hexan-5-yl)urea;
1-{1-[2-Fluoro-3-(2,2,2-trifluoro-ethoxy)-phenyl]-ethyl}-3-spiro[2.3]hex-5-yl-urea;
1-{1-[3-Fluoro-5-(2,2,2-trifluoro-ethoxy)-phenyl]-ethyl}-3-spiro[2.3]hex-5-yl-urea;
1-[1-(2-Methoxy-3-trifluoromethyl-phenyl)-ethyl]-3-spiro[2.3]hex-5-yl-urea;
1-(2-Fluoro-3-trifluoromethoxy-benzyl)-3-spiro[2.3]hex-5-yl-urea;
1-Spiro[2.3]hex-5-yl-3-{1-[3-(2,2,2-trifluoro-1-methyl-ethoxy)-phenyl]-cyclopropyl)-urea;
1-[1-(3-Difluoromethoxy-phenyl)-cyclopropyl]-3-spiro[2.3]hex-5-yl-urea;
1-[1-(2-Methoxy-5-trifluoromethyl-phenyl)-ethyl]-3-spiro[2.3]hex-5-yl-urea;
1-[3-Hydroxy-1-(3-trifluoromethoxy-phenyl)-propyl]-3-spiro[2.3]hex-5-yl-urea;
1-[1-(2-Difluoromethoxy-pyridin-4-yl)-ethyl]-3-spiro[2.3]hex-5-yl-urea;
1-Spiro[2.3]hex-5-yl-3-[1-(3-trifluoromethyl-phenyl)-ethyl]-urea;
1-Spiro[2.3]hex-5-yl-3-[1-(3-trifluoromethoxy-phenyl)-ethyl]-urea;
1-(3-Fluoro-5-trifluoromethyl-benzyl)-3-spiro[2.3]hex-5-yl-urea;
1-[2-Hydroxy-1-(3-trifluoromethyl-phenyl)-ethyl]-3-spiro[2.3]hex-5-yl-urea;
1-Spiro[2.3]hex-5-yl-3-{1-[6-(2,2,2-trifluoro-ethoxy)-pyrimidin-4-yl]-ethyl}-urea;
1-{1-[2-Methyl-6-(2,2,2-trifluoro-ethoxy)-pyrimidin-4-yl]-ethyl}-3-spiro[2.3]hex-5-yl-urea;
1-[2,2-Difluoro-1-(3-trifluoromethyl-phenyl)-ethyl]-3-spiro[2.3]hex-5-yl-urea;
1-[2-Methoxy-1-(3-trifluoromethyl-phenyl)-ethyl]-3-spiro[2.3]hex-5-yl-urea;
1-(4-Fluoro-3-trifluoromethyl-benzyl)-3-spiro[2.3]hex-5-yl-urea;
1-(2-Fluoro-3-trifluoromethyl-benzyl)-3-spiro[2.3]hex-5-yl-urea;
1-(2-Chloro-3-trifluoromethyl-benzyl)-3-spiro[2.3]hex-5-yl-urea;
1-(2-Fluoro-5-trifluoromethyl-benzyl)-3-spiro[2.3]hex-5-yl-urea;
1-{1-[5-Methoxy-6-(2,2,2-trifluoro-ethoxy)-pyrimidin-4-yl]-ethyl}-3-spiro[2.3]hex-5-yl-urea;
1-Spiro[2.3]hex-5-yl-3-(2-trifluoromethoxy-pyridin-4-ylmethyl)-urea;
1-{2-Methoxy-1-[2-(2,2,2-trifluoro-ethoxy)-pyridin-4-yl]-ethyl}-3-spiro[2.3]hex-5-yl-urea;
1-(3-Difluoromethoxy-benzyl)-3-spiro[2.3]hex-5-yl-urea;
1-Spiro[2.3]hex-5-yl-3-(3-trifluoromethoxy-benzyl)-urea;
1-Spiro[2.3]hex-5-yl-3-[3-(2,2,2-trifluoro-ethoxy)-benzyl]-urea;
1-(3-Difluoromethyl-benzyl)-3-spiro[2.3]hex-5-yl-urea;
1-Spiro[2.3]hex-5-yl-3-[2-(2,2,2-trifluoro-ethoxy)-pyridin-4-ylmethyl]-urea;
1-(6,6-Difluoro-spiro[3.3]hept-2-yl)-3-[2-(2,2,2-trifluoro-ethoxy)-pyridin-4-ylmethyl]-urea;
1-(6,6-Difluoro-spiro[3.3]hept-2-yl)-3-[1-(3-trifluoromethyl-phenyl)-ethyl]-urea;
1-Spiro[3.3]hept-2-yl-3-[(1H-[1,2,3]triazol-4-yl)-(3-trifluoromethyl-phenyl)-methyl]-urea;
1-[(1-Methyl-1H-tetrazol-5-yl)-(3-trifluoromethyl-phenyl)-methyl]-3-spiro[3.3]hept-2-yl-urea;
1-[(2-Methyl-2H-tetrazol-5-yl)-(3-trifluoromethyl-phenyl)-methyl]-3-spiro[3.3]hept-2-yl-urea;
1-[(3-Methyl-3H-[1,2,3]triazol-4-yl)-(3-trifluoromethyl-phenyl)-methyl]-3-spiro[3.3]hept-2-yl-urea;
1-[1-(2-Difluoromethoxy-pyridin-4-yl)-ethyl]-3-(6-iodo-spiro[3.3]hept-2-yl)-urea;
1-[(R)-1-(2-Difluoromethoxy-pyridin-4-yl)-ethyl]-3-spiro[3.3]hept-2-yl-urea;
1-[(S)-1-(2-Difluoromethoxy-pyridin-4-yl)-ethyl]-3-spiro[3.3]hept-2-yl-urea;
1-Spiro[3.3]hept-2-yl-3-{1-[3-((R)-2,2,2-trifluoro-1-methyl-ethoxy)-phenyl]-cyclopropyl}-urea;
1-Spiro[3.3]hept-2-yl-3-{1-[3-((S)-2,2,2-trifluoro-1-methyl-ethoxy)-phenyl]-cyclopropyl}-urea;
1-Spiro[3.3]hept-2-yl-3-[2-((R)-2,2,2-trifluoro-1-methoxymethyl-ethoxy)-pyridin-4-ylmethyl]-urea;
1-Spiro[3.3]hept-2-yl-3-[2-((S)-2,2,2-trifluoro-1-methoxymethyl-ethoxy)-pyridin-4-ylmethyl]-urea;
1-[(S)-1-(3-Difluoromethoxy-phenyl)-ethyl]-3-spiro[3.3]hept-2-yl-urea;
1-[(R)-1-(3-Difluoromethoxy-phenyl)-ethyl]-3-spiro[3.3]hept-2-yl-urea;
1-Spiro[3.3]hept-2-yl-3-[(S)-1-(3-trifluoromethyl-phenyl)-ethyl]-urea;
1-Spiro[3.3]hept-2-yl-3-{(S)-1-[3-(2,2,2-trifluoro-ethoxy)-phenyl]-ethyl}-urea;
1-{(S)-1-[4-Fluoro-3-(2,2,2-trifluoro-ethoxy)-phenyl]-ethyl}-3-spiro[3.3]hept-2-yl-urea;
1-Spiro[3.3]hept-2-yl-3-[(R)-2,2,2-trifluoro-1-(3-trifluoromethyl-phenyl)-ethyl]-urea;
1-Spiro[3.3]hept-2-yl-3-[(R)-(1H-[1,2,4]triazol-3-yl)-(3-trifluoromethyl-phenyl)-methyl]-urea;
1-Spiro[3.3]hept-2-yl-3-[2-((R)-2,2,2-trifluoro-1-methyl-ethoxy)-pyrimidin-4-ylmethyl]-urea;
1-Spiro[3.3]hept-2-yl-3-[2-((S)-2,2,2-trifluoro-1-methyl-ethoxy)-pyrimidin-4-ylmethyl]-urea;
1-Spiro[3.3]hept-2-yl-3-{(S)-1-[6-(2,2,2-trifluoro-ethoxy)-pyrimidin-4-yl]-ethyl}-urea;
1-Spiro[3.3]hept-2-yl-3-{(R)-1-[6-(2,2,2-trifluoro-ethoxy)-pyrimidin-4-yl]-ethyl}-urea;
1-[(R)-2-Hydroxy-1-(3-trifluoromethyl-phenyl)-ethyl]-3-spiro[3.3]hept-2-yl-urea;
1-[(S)-2-Hydroxy-1-(3-trifluoromethyl-phenyl)-ethyl]-3-spiro[3.3]hept-2-yl-urea;
1-[(R)-2-Hydroxy-1-(3-trifluoromethyl-phenyl)-ethyl]-3-spiro[2.3]hex-5-yl-urea;
1-[(R)-1-(2-Difluoromethoxy-pyridin-4-yl)-2-methoxy-ethyl]-3-spiro[3.3]hept-2-yl-urea;
1-{(R)-2-Methoxy-1-[2-(2,2,2-trifluoro-ethoxy)-pyridin-4-yl]-ethyl}-3-spiro[2.3]hex-5-yl-urea;
1-[(S)-1-(2-Difluoromethoxy-pyridin-4-yl)-ethyl]-3-spiro[2.3]hex-5-yl-urea; and
1-[(S)-1-(2-Bromo-5-trifluoromethyl-phenyl)-ethyl]-3-spiro[3.3]hept-2-yl-urea;

and to the salts (in particular pharmaceutically acceptable salts) of such compounds.

It is to be understood for any of the above listed compounds, that a stereogenic center, which is not specifically assigned, may be in absolute (R)- or absolute (S)-configuration; for example a compound listed as 1-{1-[2-Methyl-6-(2,2,2-trifluoro-ethoxy)-pyrimidin-4-yl]-ethyl}-3-spiro[3.3]hept-2-yl-urea may be (S)-1-{1-[2-Methyl-6-(2,2,2-trifluoro-ethoxy)-pyrimidin-4-yl]-ethyl}-3-spiro[3.3]hept-2-yl-urea, (R)-1-{1-[2-Methyl-6-(2,2,2-trifluoro-ethoxy)-pyrimidin-4-yl]-ethyl}-3-spiro[3.3]hept-2-yl-urea or any mixture thereof.

Where the plural form is used for compounds, salts, pharmaceutical compositions, diseases or the like, this is intended to mean also a single compound, salt, disease or the like.

Any reference to a compound of Formula (I) as defined in any one of embodiments 1) to 37) is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quere (Eds.), RSC Publishing, 2012.

The present invention also includes isotopically labelled, especially ²H (deuterium) labelled compounds of Formula (I), which compounds are identical to the compounds of Formula (I) except that one or more atoms have each been replaced by an atom having the same atomic number but an atomic mass different from the atomic mass usually found in nature. Isotopically labelled, especially ²H (deuterium) labelled compounds of Formula (I) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of Formula (I) are not isotopically labelled, or they are labelled only with one or more deuterium atoms. In a sub-embodiment, the compounds of Formula (I) are not isotopically labelled at all. Isotopically labelled compounds of Formula (I) may be prepared in analogy to the methods described hereinafter, but using the appropriate isotopic variation of suitable reagents or starting materials.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.

The compounds of formula (I) as defined in any one of embodiments 1) to 37) and their pharmaceutically acceptable salts can be used as medicaments, e.g. in the form of pharmaceutical compositions for enteral (such especially oral) or parenteral administration (including topical application or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of Formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention/prophylaxis or treatment of a disease or disorder mentioned herein comprising administering to a subject a pharmaceutically active amount of a compound of Formula (I) as defined in any one of embodiments 1) to 37).

For avoidance of any doubt, if compounds are described as useful for the prevention or treatment of certain diseases, such compounds are likewise suitable for use in the preparation of a medicament for the prevention or treatment of said diseases.

Another aspect of the invention concerns a method for the prevention/prophylaxis or the treatment of a disease or disorder as mentioned below in a patient comprising the administration to said patient of a pharmaceutically active amount of a compound of Formula (I) as defined in any one of embodiments 1) to 37) or a pharmaceutically acceptable salt thereof.

The compounds according to Formula (I) as defined in any one of embodiments 1) to 37) are useful for the prevention/prophylaxis or treatment of diseases or disorders associated with KCNQ2, KCNQ3, KCNQ4, KCNQ5 and/or diseases or disorders associated with mutations in KCNQ2, KCNQ3, KCNQ4, KCNQ5.

Such diseases or disorders associated with KCNQ2, KCNQ3, KCNQ4, KCNQ5 and/or diseases or disorders associated with mutations in KCNQ2, KCNQ3, KCNQ4, KCNQ5 may in particular be defined as comprising epilepsy, myokymia, tinnitus, neuropathic and inflammatory pain, psychiatric disorders, and diseases affecting the smooth muscles.

Epilepsy may be defined as comprising:
epilepsy with focal onset seizures (with or without impaired awareness, with motor or nonmotor onset symptoms);
(epilepsy with generalized seizures with motor onset symptoms;
epilepsy with unknown seizure onset;
epilepsy with traumatic brain injury-induced seizures;
neonatal epilepsy including early onset epileptic encephalopathy with or without neurodevelopmental impairment (such as Othahara syndrome, early infantile epileptic encephalopathy, early myoclonic encephalopathy, epilepsy with suppression-burst pattern, benign or self-limiting familial neonatal epilepsy);
infantile/childhood epilepsy syndromes including epilepsy with neurodevelopmental impairment, focal epilepsies of childhood and idiopathic epilepsy syndromes.

Diseases affecting the smooth muscles may be defined as comprising diseases affecting the visceral smooth muscles (such as functional dyspepsia, irritable bowel syndrome and overactive bladder), diseases affecting the vascular smooth muscles (such as hypertension, and cerebral vasospasm), diseases affecting the airway smooth muscles (such as asthma and chronic obstructive pulmonary disease) and hearing disorders.

Psychiatric disorders may be defined as comprising anxiety, schizophrenia, mania, and autism.

Especially, compounds of Formula (I) according to any one of embodiments 1) to 37), or pharmaceutically acceptable salts thereof, are suitable for the prevention/prophylaxis or treatment of epilepsy; and especially of epilepsy with focal seizures, epilepsy with generalized seizures, epilepsy with unknown onset, neonatal epilepsy, and/or infantile/childhood epilepsy syndromes with or without neurodevelopmental decline.

Preparation of Compounds of Formula (I)

A further aspect of the invention is a process for the preparation of compounds of Formula (I). Compounds according to Formula (I) of the present invention can be prepared from commercially available or well known starting materials according to the methods described in the experimental part; by analogous methods; or according to the general sequence of reactions outlined below, wherein R¹, R^{2A}, R^{2B}, R³, R⁴, X¹, X², X³, and Y are as defined for Formula (I). Other abbreviations used herein are explicitly defined, or are as defined in the experimental section. In some instances the generic groups R¹, R^{2A}, R^{2B}, R³, R⁴, X¹, X², X³, and Y might be incompatible with the assembly illustrated in the schemes below and so will require the use of protecting groups (PG). The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups as necessary are in place. The compounds obtained may also be converted into salts, especially pharmaceutically acceptable salts thereof in a manner known per se.

General Preparation Routes:

Generally, compounds of Formula I can be synthesised by treating an amine of Structure 2 (or the corresponding salt, like HCl or TFA salts) with an isocyanate 3 in the presence of a base such as NEt₃ or DIPEA in solvent such as DCM or MeCN. Alternatively, an isocyanate of Structure 4 can be reacted with an amine 5 (or the corresponding salt, like HCl or TFA salts) in the presence of a base such as NEt₃ or DIPEA in solvent such as DCM or MeCN to afford compounds of Formula I-A (Scheme 1).

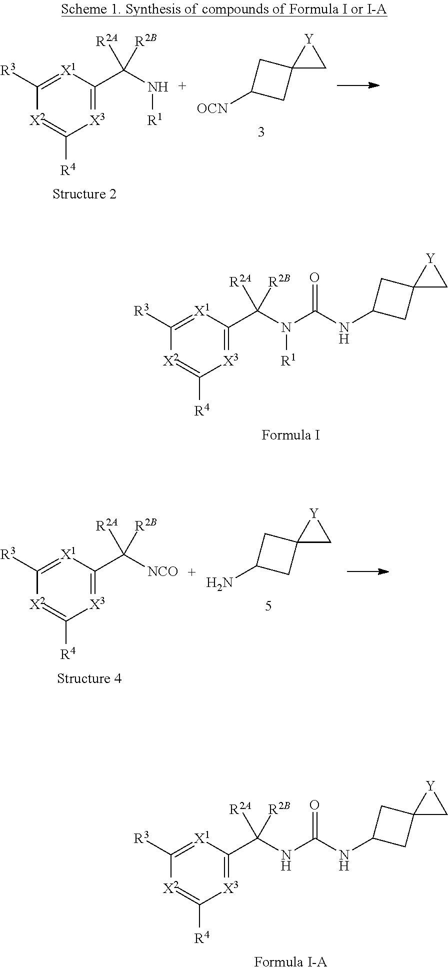

Alternatively, an amine of Structure 2 (or the corresponding salt, like HCl or TFA salts) is condensed with 4-nitrophenyl chloroformate in the presence of a base like NEt₃ or DIPEA to give a carbamate 6 (Scheme 2). The carbamate 6 is then treated with an amine 5 (or the corresponding salt, like HCl or TFA salts) in the presence of a base like NEt₃ in a solvent like THF to yield a compound of Formula I. The sequence can also start by first reacting an amine (or the corresponding salt, like HCl or TFA salts) with 4-nitrophenyl chloroformate in the presence of a base like NEt₃ or DIPEA to give a carbamate 7 (Scheme 2). The carbamate 7 is then treated with an amine of Structure 2 (or the corresponding salt, like HCl or TFA salts) in the presence of a base like NEt₃ in solvent like THF to yield a compound of Formula I.

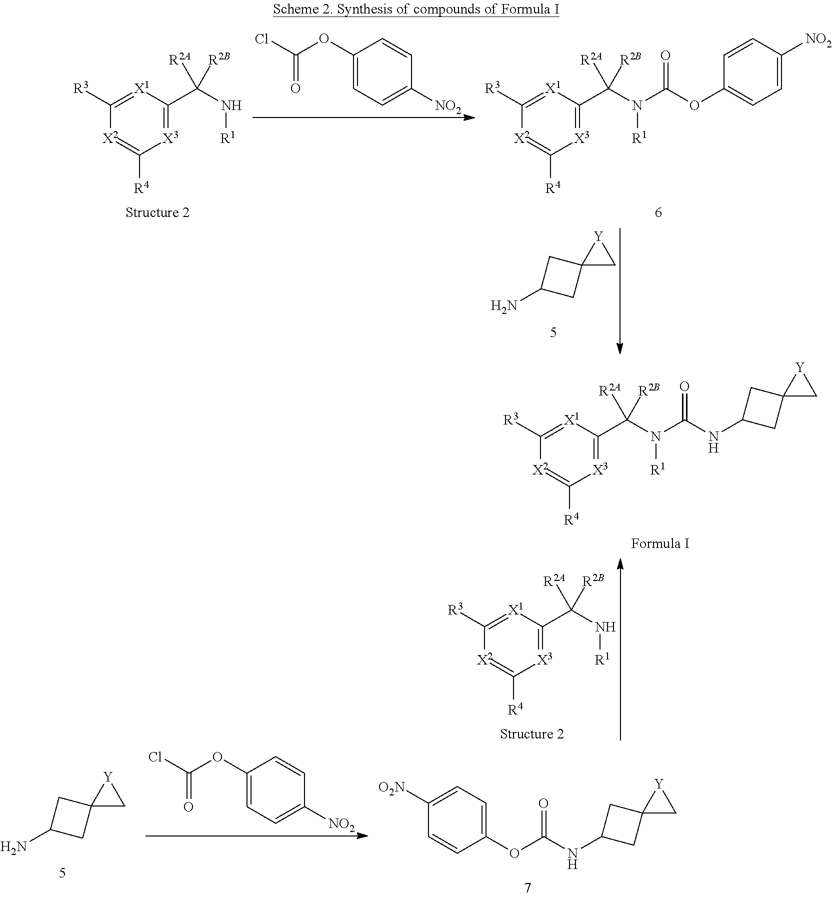

In another aspect, an amine of Structure 2 (or the corresponding salt, like HCl or TFA salts) is activated with an activating agent like CDI, triphosgene, or trifluoroethoxycarbonate and the activated intermediate is in-situ treated with an amine 5 (or the corresponding salt, like HCl or TFA salts) in the presence of a base like NEt₃ or DIPEA to yield a compound of Formula I (Scheme 3). Conversely, an amine 5 (or the corresponding salt, like HCl or TFA salts) can be activated with an activating agent like CDI, triphosgene, or trifluoroethoxycarbonate and the activated intermediate is in-situ treated with an amine of Structure 2 (or the corresponding salt, like HCl or TFA salts) in the presence of a base like NEt₃ or DIPEA to yield a compound of Formula I.

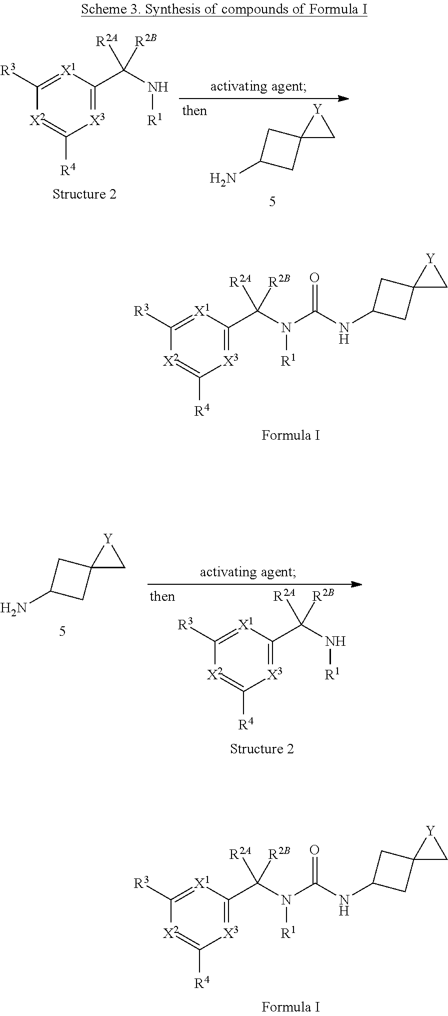

Amines of Structure 2-A or 2-B can be synthesized by taking advantage of the Ellman's auxiliary (Scheme 4). Thus, an aldehyde 8 is treated with tert-butanesulfinamide 9 in the presence of Ti(OEt)₄ to provide a tert-butanesulfinyl imine 10. Compound 10 is then treated with a nucleophile such as a Grignard reagent 11 to afford a protected amine 12. The tert-butanesulfinyl group is then cleaved under mild acidic conditions like HCl in MeOH to afford an amine of Structure 2-A (or the corresponding HCl salt). Alternatively, imine 10 can be reduced with a reducing agent like NaBH₄ in MeOH to yield a protected amine 13. The tert-butanesulfinyl group is then cleaved under mild acidic conditions like HCl in MeOH to afford an amine of Structure 2-B (or the corresponding HCl salt). Alternatively, a ketone 14 can be reacted with tert-butanesulfinamide 9 in the presence of Ti(OEt)₄ to provide a tert-butanesulfinyl imine 15. Compound 15 is then treated with a Grignard or lithiated reagent 16 to afford a protected amine 17. The tert-butanesulfinyl group is then cleaved under mild acidic conditions like HCl in MeOH to afford an amine of Structure 2-C (or the corresponding HCl salt).

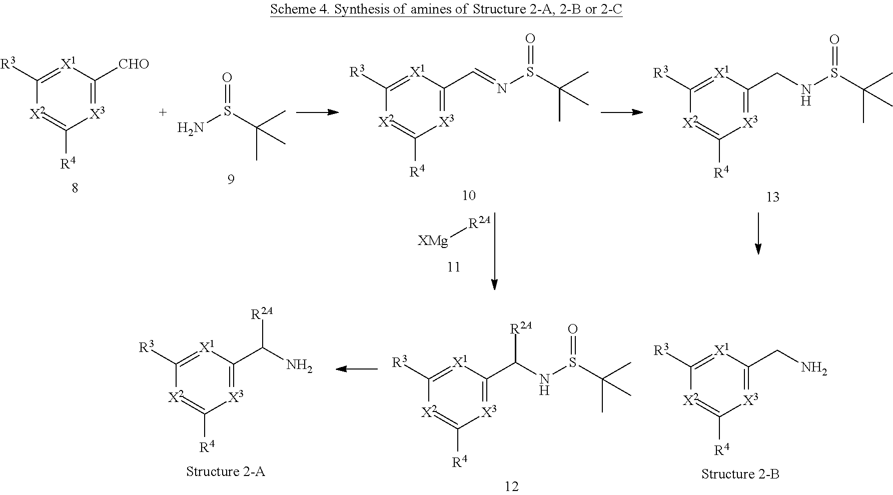

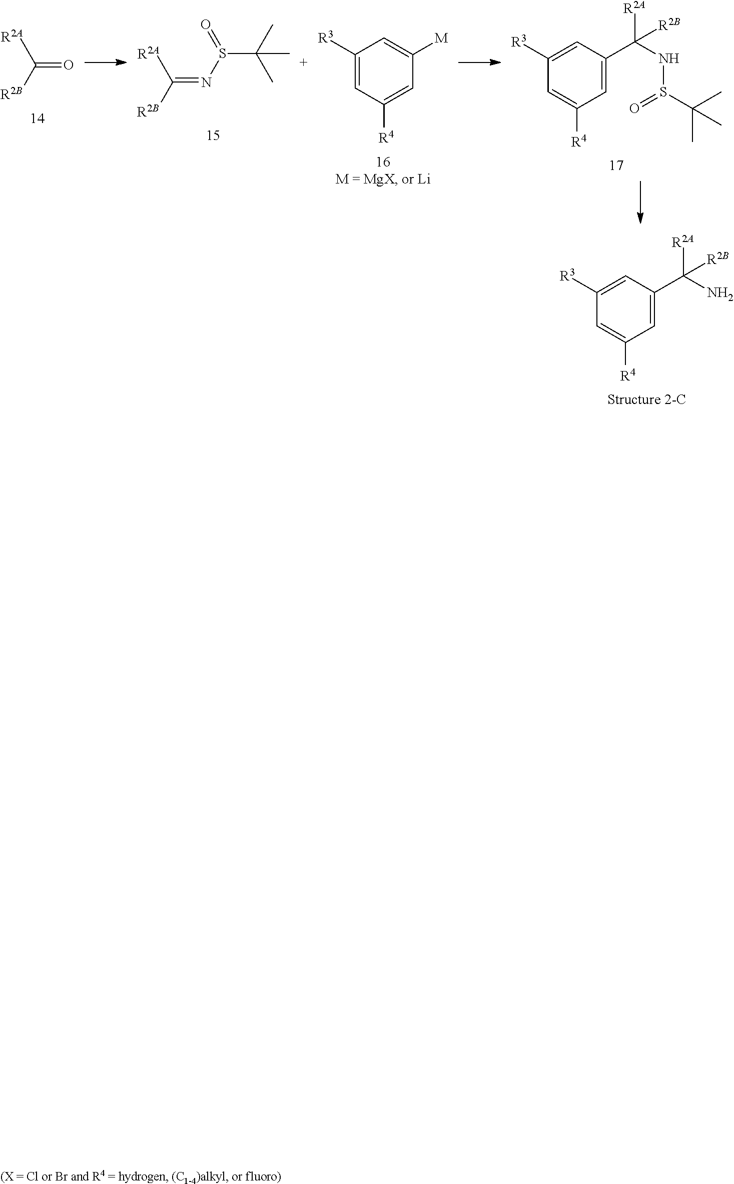

In another aspect, an amine of Structure 2-A can be synthesized using photoredox catalysis (Scheme 5). A bromide 18 is reacted with a Boc-protected amino acid 19 in the presence of an iridium catalyst like [Ir{dF(CF₃)ppy}₂(dtbpy)]PF₆ and a nickel catalyst like NiCl₂.glyme in a solvent like DMSO or DMA under blue LED irradiation to give a Boc-protected amine 20 (Science 2014, 345, 437-440). The Boc-protecting group is then cleaved under acidic conditions like TFA in DCM or 4M HCl in dioxane to give an amine of Structure 2-A (or the corresponding salt, like HCl or TFA salts).

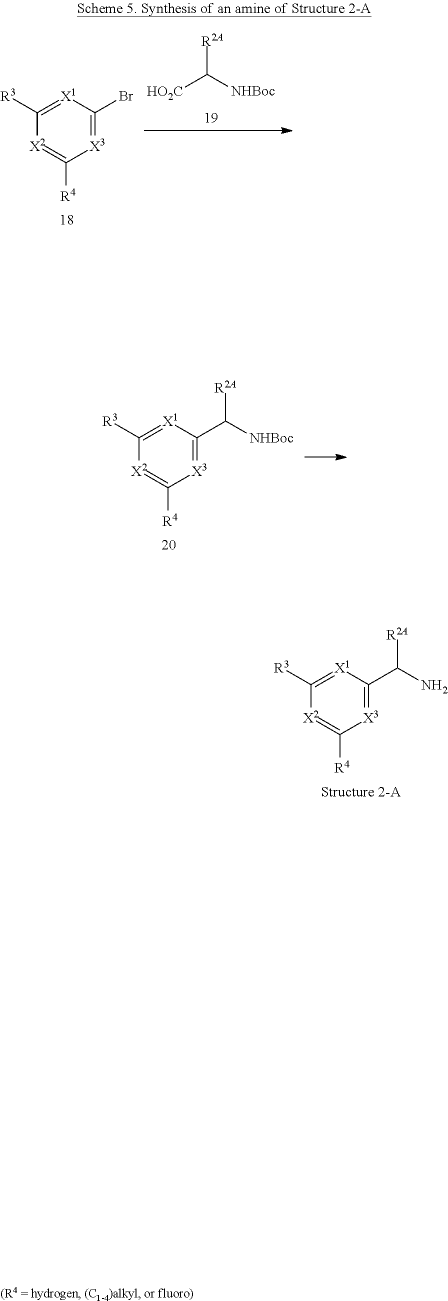

An amine of Structure 2-B can also be obtained from the corresponding nitrile 21 (Scheme 6). A solution of a nitrile 21 in MeOH can be reduced using a catalyst like Ra/Ni under an H₂-atmosphere (in flow or batch mode) or LiAlH₄ in a solvent like THE to give an amine of Structure 2-B. Alternatively, nitrile 21 can be reduced using a nickel catalyst like NiCl₂.6H₂O and NaBH₄ in the presence of Boc₂O to give a Boc-protected amine 22. Deprotection under acidic conditions like TFA in DCM or HCl in dioxane yield an amine of Structure 2-B (or the corresponding HCl or TFA salt). Nitrile 21 can also be converted to the corresponding ketones 23 using MeMgBr in a solvent like Et₂O. Ketone 23 can undergo a reductive amination in a solvent like MeOH with for example ammonium acetate and sodium cyanoborohydride to give an amine of Structure 2-A (where R^{2A} is methyl). Nitrile 21 can also be subjected to a Kulinkovich reaction in Et₂O using EtMgBr in the presence of a titanium salt like Ti(OiPr)₄ and borontrifluoride to give an amine of Structure 2-D. Finally, nitrile 21 can react with a Boc-protected amino acid in the presence of cesium fluoride and an iridium catalyst like Ir(p-F(t-Bu)-ppy)₃ in a solvent like DMSO or DMA under blue LED irradiation to give a Boc-protected amine 24 (JACS 2014, 136, 5257-5260). The protecting group can then be cleaved under acidic conditions like TFA in DCM or HCl in dioxane to give an amine of Structure 2-E (or the corresponding HCl or TFA salt).

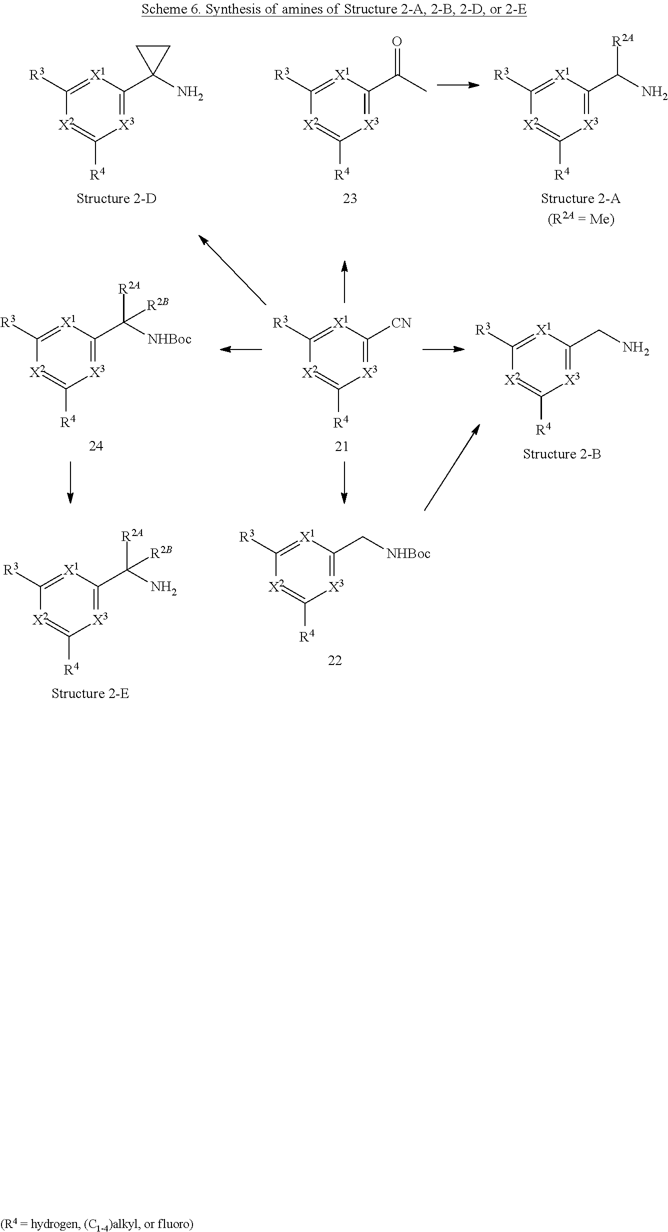

Aldehydes 8-A can be prepared as described in Scheme 7. Thus, alcohol 25 can be reacted with an alkylating agent like alkylsulfonate, alkylbromide, or alkyliodide in the presence of a base like Cs₂CO₃ or K₂CO₃ in a solvent like DMF to give an aldehyde 8-A. Similarly, an alcohol 26 can be converted into the corresponding bromide 18-A.

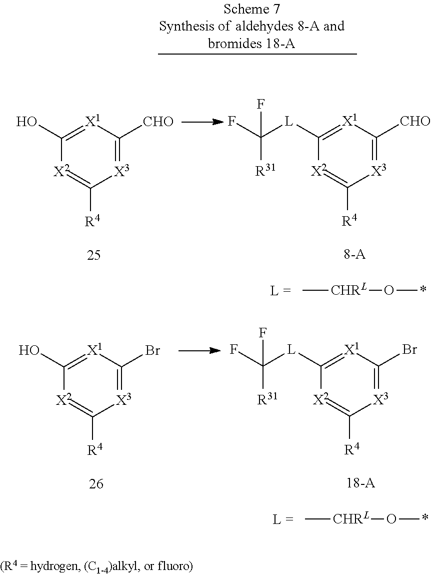

Nitriles 21-A are obtained through a S_{N}Ar reaction between a chloro or fluoro nitrile 27 and an alcohol like trifluoroethanol in the presence of a base like sodium hydride in a solvent like THE (Scheme 8). Alternatively, nitrile 27 can undergo a S_{N}Ar reaction with an amine (or the corresponding HCl salt) in a solvent like NMP and a base like NEt₃ under microwave irradiation to yield a nitrile 21-B. Finally, a cyanation between chloro or bromo derivative 28 and ZnCN₂ in the presence of a palladium catalyst like Pd₂(dba)₃ and a ligand like dppf in a solvent like DMF give a nitrile 21.

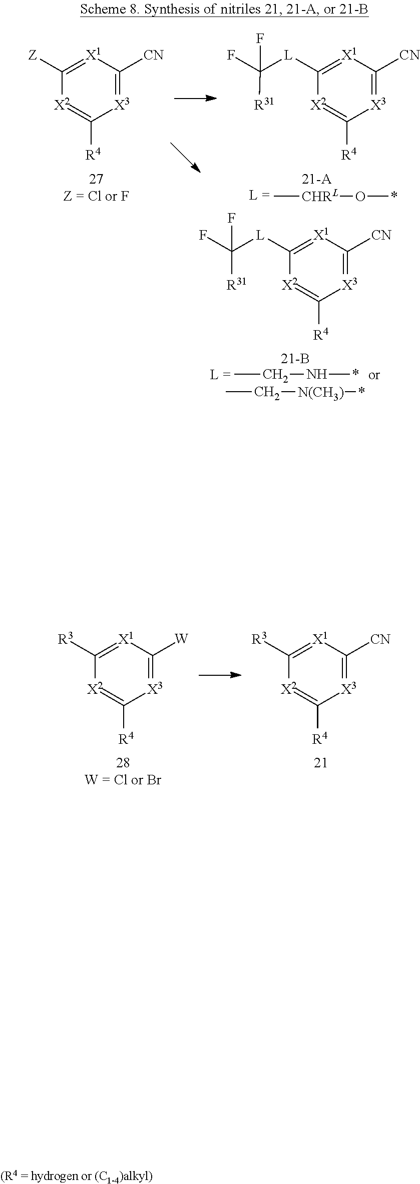

An amine of Structure 2 can also be prepared by methods described in Scheme 9. Thus, a Boc-protected amine 30 can be treated with an alkylating agent like alkyl bromide or alkyl iodide in the presence of a base or a silver salt like Ag₂O to give a Boc-protected amine 31. The Boc-protecting group is then cleaved under acidic conditions like TFA in DCM or 4M HCl in dioxane to give an amine of Structure 2 (or the corresponding salt, like HCl or TFA salt). Alternatively, an aldehyde 8 can undergo a reductive amination with an amine 32 in a solvent like DCM and in the presence of a reducing agent like NaHB(OAc)₃ and a base like DIPEA to give an amine of Structure 2, wherein R^{2A} and R^{2B} represent hydrogen.

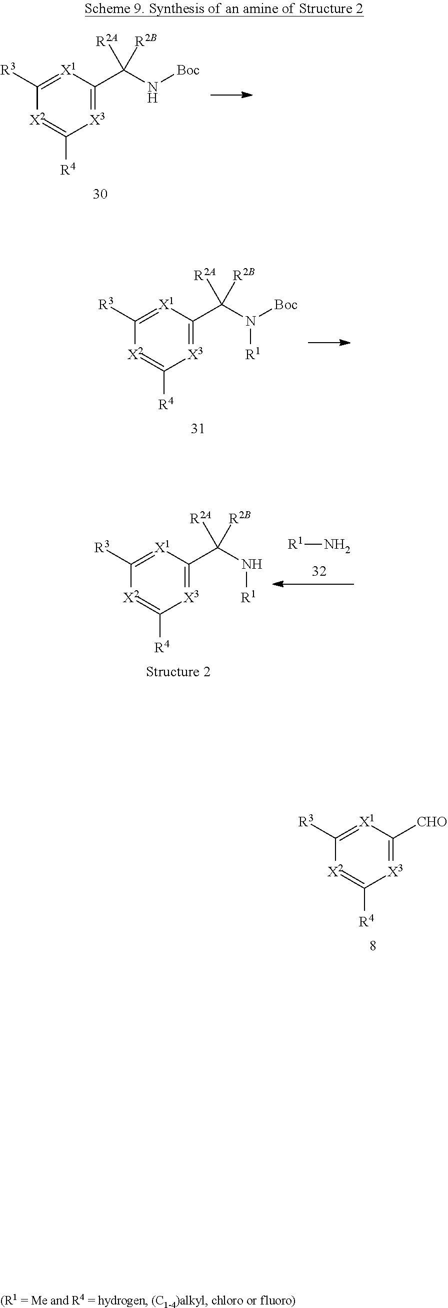

### EXPERIMENTAL SECTION

### Abbreviations (as Used Herein and in the Description Above)

anh. anhydrous
Ac acetyl
aq. aqueous
Boc tert.-butyloxycarbonyl
Bu butyl
CD 1,1′-carbonyldiimidazole
comb. Combined
dba dibenzylideneacetone
DCM dichloromethane
DIPEA N-ethyldiisopropylamine
DMA dimethylacetamide
DMF dimethylformamide
DMSO dimethylsulfoxide
dppf 1,1′-ferrocenediyl-bis(diphenylphosphine)
eq equivalent
Et ethyl
FBS fetal bovine serum
FLIPR Fluorescent imaging plate reader
Fluo-8-AM bis(acetoxymethyl) 2,2′-((4-(6-(acetoxymethoxy)-3-oxo-3H-xanthen-9-yl)-2-(2-(bis(2-acetoxymethoxy)-2-oxoethyl)amino)phenoxy)ethoxy)phenyl)azanediyl)diacetate
h hour(s)
HATU 2-(7-Aza-1H-benzotriazole-1-yl)-1,1,3,3-tetramethyluronium hexafluorophosphate
HBSS Hank's balanced salt solution
HEK293 Human embryonic kidney 293 cells
HEPES 4-(2-hydroxyethyl)-piperazine-1-ethanesulfonic acid
Hept heptanes
HV High vacuum
HPLC high performance liquid chromatography
iPr isopropyl
Ir(p-F(t-Bu)-ppy)₃ tris (2-(3-tert-butylphenyl)-4-tert-butylpyridine) iridium
[Ir{dF(CF₃)ppy₂(dtbpy)]PF₆ [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
LC liquid chromatography
LED light-emitting diode
M molarity [mol L⁻¹]
Me methyl
MS mass spectroscopy
min minute(s)
N normality
NMDA N-methyl-D-aspartate
NMP N-methyl-2-pyrrolidone
org. organic
PBS phosphate-buffered saline
PG protecting group
Ph phenyl
Prep. preparative
rpm rotations per minute
rt room temperature
sat. saturated
sec second(s)
SFC supercritical fluid chromatography
soln. solution
TBME tert-butyl methyl ether
tBu tert-butyl
TFA trifluoroacetic acid
THE tetrahydrofuran
t_{R} retention time
UPLC Ultra performance liquid chromatography
UV ultraviolet
XE-991 10,10-bis(4-pyridinylmethyl)-9(10H)-anthracenone

I. Chemistry

The following examples illustrate the preparation of biologically active compounds of the invention but do not at all limit the scope thereof.

General remarks: All solvents and reagents are used as obtained from commercial sources unless otherwise indicated. Temperatures are indicated in degrees Celsius (° C.). Unless otherwise indicated, the reactions take place at room temperature (rt) under an argon or nitrogen atmosphere and are run in a flame dried round-bottomed flask equipped with a magnetic stir bar. In mixtures, relations of parts of solvent or eluent or reagent mixtures in liquid form are given as volume relations (v/v), unless indicated otherwise.

Characterization Methods Used:

LC-MS 1

LC-MS-conditions: Analytical. Pump: Waters Acquity Binary, Solvent Manager, MS: Waters SQ Detector or Xevo TQD, DAD: Acquity UPLC PDA Detector. Column: Acquity UPLC CSH C18 1.7 um, 2.1×50 mm from Waters, thermostated in the Acquity UPLC Column Manager at 60° C. Eluents: A1: H₂O+0.05% formic acid; B1: MeCN+0.045% formic acid. Method: Gradient: 2% B to 98% B over 2.0 min. Flow: 1.0 mL/min. Detection at 214 nm and MS, retention time t_{R} is given in min.

LC-MS 2 to 3

UPLC/MS analyses are performed on Acquity UPLC setup. The column temperature is 40° C.

The LC retention times are obtained using the following elution conditions:
LC-MS 2: Analytical UPLC on a Agilent Zorbax RRHD SB-Aq (2.1×50 mm, 1.8 um); detection at 210 nM and MS; Gradient of water/0.04% TFA (A) and MeCN (B). The eluent flow rate was 0.8 mL/min and the characteristics of the eluting mixture proportion in function of the time t from start of the elution are summarized in the table below (a linear gradient being used between two consecutive time points):

t (min)

| | 0 | 1.2 | 1.9 | 2.1 |
|---|---|---|---|---|
| Solvent A (%) | 95 | 5 | 5 | 95 |
| Solvent B (%) | 5 | 95 | 95 | 5 |

LC-MS 3: Analytical UPLC on a Waters BEH C18 (2.1×50 mm, 2.5 um); detection at 210 nM and MS; Gradient of water/0.04% NH₃ [c(NH₃)=13 mmol/l] (A) and MeCN (B). The eluent flow rate was 0.8 mL/min and the characteristics of the eluting mixture proportion in function of the time t from start of the elution are summarized in the table below (a linear gradient being used between two consecutive time points):

t (min)

| | 0 | 1.2 | 1.9 | 2.1 |
|---|---|---|---|---|
| Solvent A (%) | 95 | 5 | 5 | 95 |
| Solvent B (%) | 5 | 95 | 95 | 5 |

Preparative LC-MS Methods Used:

Preparative HPLC/MS purifications are performed on a Gilson HPLC system, equipped with a Gilson 215 autosampler, Gilson 333/334 pumps, Finnigan AQA MS detector system, and a Dionex UV detector, using a Waters Xbridge C18 or an Waters Atlantis column, with a linear gradient of water/formic acid 0.02% (A) and MeCN (B) (acidic conditions) or water/ammonia 0.02% (A) and MeCN (B) (basic conditions).

Combiflash

Flash column chromatography was performed using a combiflash from Teledyne ISCO.

Preparative Chiral HPLC/SFC Methods Used:

Preparative chiral HPLC purifications are performed on a HPLC system equipped with a Gilson 215 autosampler, Varian SD-1 pumps and a Dionex UV detector. Following parameters were used:

Preparative chiral HPLC 1: A chiral ChiralPak AY-H column (30×250 mm, 5 um) was used. The following parameters were used: Hept/EtOH+0.1% DEA 9:1, flow 34 mL/min.

Preparative chiral SFC purifications are performed on a Sepiatec Prep SFC 360 system.

Following parameters were used:

Preparative chiral SFC 1: A ChiralPak AS-H column (30×250 mm, 5 um) was used. The modifier was EtOH (10%), run for 12 min and at a flow rate of 160 mL/min. The following system settings were used: backpressure 100 bar, temperature pumphead 5° C., temperature fraction module 20° C., and temperature column department 40° C.

Preparative chiral SFC 2: A ChiralCel OZ-H column (30×250 mm, 5 um) was used. The modifier was EtOH (15%), run for 4 min and at a flow rate of 160 mL/min. The following system settings were used: backpressure 100 bar, temperature pumphead 4° C., temperature fraction module 20° C., and temperature column department 40° C.

Preparative chiral SFC 3: A Chiralpak AY-H column (30×250 mm, 5 um) was used. The modifier was EtOH (10%), run for 10 min and at a flow rate of 160 mL/min. The following system settings were used: backpressure 100 bar, temperature pumphead 5° C., temperature fraction module 20° C., and temperature column department 40° C.

Preparative chiral SFC 4: A (R,R) Whelk-01 column (30×250 mm, 5 um) was used. The modifier was EtOH (50%), run for 2 min and at a flow rate of 160 mL/min. The following system settings were used: backpressure 100 bar, temperature pumphead 5° C., temperature fraction module 20° C., and temperature column department 40° C.

Preparative chiral SFC 5: A ChiralPak AS-H column (30×250 mm, 5 um) was used. The modifier was EtOH/MeCN 1:1 (10%), run for 17 min and at a flow rate of 160 mL/min. The following system settings were used: backpressure 120 bar, temperature pumphead 5° C., temperature fraction module 15° C., and temperature column department 40° C.

Preparative chiral SFC 6: A (R,R) Whelk-01 column (30×250 mm, 5 um) was used. The modifier was EtOH (25%), run for 2.3 min and at a flow rate of 160 mL/min. The following system settings were used: backpressure 100 bar, temperature pumphead 5° C., temperature fraction module 20° C., and temperature column department 40° C.

Preparative chiral SFC 7: A ChiralPak IC column (30×250 mm, 5 um) was used. The modifier was EtOH (20%), run for 2.5 min and at a flow rate of 160 mL/min. The following system settings were used: backpressure 100 bar, temperature pumphead 5° C., temperature fraction module 20° C., and temperature column department 40° C.

Preparative chiral SFC 8: A ChiralPak IC column (30×250 mm, 5 um) was used. The modifier was iPrOH (25%), run for 5 min and at a flow rate of 160 mL/min. The following system settings were used: backpressure 100 bar, temperature pumphead 5° C., temperature fraction module 20° C., and temperature column department 40° C.

Preparative chiral SFC 9: A ChiralPak IF column (30×250 mm, 5 um) was used. The modifier was EtOH (35%), run for 2.5 min and at a flow rate of 160 mL/min. The following system settings were used: backpressure 100 bar, temperature pumphead 5° C., temperature fraction module 20° C., and temperature column department 40° C.

Preparative chiral SFC 10: A ChiralCel OD-H column (30×250 mm, 5 um) was used. The modifier was EtOH (15%), run for 3.5 min and at a flow rate of 160 mL/min. The following system settings were used: backpressure 100 bar, temperature pumphead 5° C., temperature fraction module 20° C., and temperature column department 40° C.

Preparative chiral SFC 11: A ChiralPak IH column (30×250 mm, 5 um) was used. The modifier was MeOH (25%), run for 2.5 min and at a flow rate of 160 mL/min. The following system settings were used: backpressure 100 bar, temperature pumphead 5° C., temperature fraction module 20° C., and temperature column department 40° C.

Preparative chiral SFC 12: A ChiralPak AD-H column (30×250 mm, 5 um) was used. The modifier was EtOH (15%), run for 4 min and at a flow rate of 160 mL/min. The following system settings were used: backpressure 100 bar, temperature pumphead 5° C., temperature fraction module 20° C., and temperature column department 40° C.

Preparative chiral SFC 13: A ChiralPak IH column (30×250 mm, 5 um) was used. The modifier was EtOH (20%), run for 3 min and at a flow rate of 160 mL/min. The following system settings were used: backpressure 100 bar, temperature pumphead 5° C., temperature fraction module 20° C., and temperature column department 40° C.

Preparative chiral SFC 14: A (R,R) Whelk-01 column (30×250 mm, 5 um) was used. The modifier was EtOH (45%), run for 2.5 min and at a flow rate of 160 mL/min. The following system settings were used: backpressure 100 bar, temperature pumphead 5° C., temperature fraction module 20° C., and temperature column department 40° C.

### Example 1: 1-Spiro[3.3]hept-2-yl-3-(3-trifluoromethyl-benzyl)-urea

To an ice-cooled solution of spiro[3.3]heptan-2-amine hydrochloride (31 mg, 0.20 mmol, 1.0 eq) and NEt₃ (34 μL, 0.24 mmol, 1.2 eq) in MeCN (1 mL), 1-(isocyanatomethyl)-3-(trifluoromethyl)benzene (42 mg, 0.2 mmol, 1.0 eq) was added. The mixture was stirred at rt for 18 hours. Water was added. The mixture was filtered and the filtrate was purified by prep. HPLC (column: Waters X-bridge, 19×50 mm, 10 um, UV/MS, basic conditions). LC-MS (1): t_{R}=1.15 min; [M+H]⁺: 313.1.

### Example 2: 1-Spiro[2.3]hex-5-yl-3-(3-trifluoromethyl-benzyl)-urea

The product was synthesized using spiro[2.3]hex-5-ylamine hydrochloride and following the procedure described in Example 1. LC-MS (1): t_{R}=1.07 min; [M+H]⁺: 299.1.

### Example 3: 1-(1,1-Difluoro-spiro[2.3]hex-5-yl)-3-(3-trifluoromethyl-benzyl)-urea

To a solution of 1,1-difluorospiro[2.3]hexan-5-amine hydrochloride (433 mg, 2.42 mmol, 1.1 eq) and NEt₃ (1.24 mL, 8.82 mmol, 4.0 eq) in THF (16 mL), 4-nitrophenyl (3-(trifluoromethyl)benzyl)carbamate (750 mg, 2.20 mmol, 1.0 eq) was added portionwise. The resulting mixture was stirred at rt for 2 h. The reaction was diluted with water (50 mL) and EtOAc (50 mL). The layers were separated. The aq. phase was extracted with EtOAc (2×50 mL). The comb. org. phases were washed with sat. aq. NaCl soln. (2×50 mL), dried over MgSO₄ and concentrated in vacuo. The residue was purified by prep. HPLC (column: Zorbax, 19×50 mm, 10 um, UV/MS, acidic conditions). LC-MS (1): t_{R}=1.06 min; [M+H]⁺: 335.0.

### Example 4: 1-(1,1-Difluoro-spiro[2.3]hex-5-yl)-3-(3-trifluoromethyl-benzyl)-urea (isomer 1) and Example 5: 1-(1,1-Difluoro-spiro[2.3]hex-5-yl)-3-(3-trifluoromethyl-benzyl)-urea (isomer 2)

1-(1,1-Difluoro-spiro[2.3]hex-5-yl)-3-(3-trifluoromethyl-benzyl)-urea was separated by the preparative chiral SFC 1 method to give Example 4 (first eluting, t_{R}=3.8 min) and Example 5 (second eluting, t_{R}=5.6 min). Example 4 (LC-MS (1): t_{R}=1.06 min; [M+H]⁺: 335.0) and Example 5 (LC-MS (1): t_{R}=1.06 min; [M+H]⁺: 335.0).

### Example 6: 1-(6,6-Difluoro-spiro[3.3]hept-2-yl)-3-(3-trifluoromethyl-benzyl)-urea

The product was synthesized using 6,6-difluorospiro[3.3]heptan-2-amine hydrochloride and following the procedure described in Example 3. LC-MS (1): t_{R}=1.08 min; [M+H]⁺: 349.1.

### Example 7: 1-[1-(3-Difluoromethoxy-phenyl)-cyclopropyl]-3-spiro[3.3]hept-2-yl-urea

To a solution of 1-(3-(difluoromethoxy)phenyl)cyclopropanamine hydrochloride (20 mg, 0.09 mmol, 1 eq) and NEt₃ (48 μL, 0.34 mmol, 4 eq) in THF (1 mL), 4-nitrophenyl spiro[3.3]heptan-2-ylcarbamate (24 mg, 0.09 mmol, 1 eq) was added. The mixture was stirred at rt overnight and then were concentrated in vacuo. The residue was purified by prep. HPLC (column: Waters X-Bridge, 30×75 mm, 10 um, UV/MS, basic conditions). LC-MS (1): t_{R}=1.14 min; [M+H]⁺: 337.1.

Example 8 to Example 11 were synthesized using the appropriate amine or amine salt derivative and following the procedure described in Example 7. LC-MS data of Example 8 to Example 11 are listed in the table below. The LC-MS conditions used were LC-MS (1).

| Example No | Name | t_{R} | [M + H]⁺ |
|---|---|---|---|
| 8 | 1-(2-Fluoro-3-trifluoromethoxy-benzyl)-3-spiro[3.3]hept- | 1.19 | 347.1 |
| | 2-yl-urea | | |
| 9 | (±)-1-[2-Methoxy-1-(3-trifluoromethyl-phenyl)-ethyl]-3- | 1.17 | 357.1 |
| | spiro[3.3]hept-2-yl-urea | | |
| 10 | (±)-1-[1-(2-Difluoromethoxy-pyridin-4-yl)-2-methoxy- | 1.07 | 356.1 |
| | ethyl]-3-spiro[3.3]hept-2-yl-urea | | |
| 11 | (±)-1-Spiro[3.3]hept-2-yl-3-(5-trifluoromethoxy-2,3- | 1.21 | 357.1 |
| | dihydro-benzofuran-3-yl)-urea | | |

### Example 12: 1-(2-Fluoro-3-trifluoromethyl-benzyl)-3-spiro[3.3]hept-2-yl-urea

To a solution of 2-fluoro-3-(trifluoromethyl)benzylamine (19 mg, 0.10 mmol, 1.0 eq) and NEt₃ (70 μL, 0.50 mmol, 5.0 eq) in MeCN (1 mL), 2-isocyanatospiro[3.3]heptane (15 mg, 0.11 mmol, 1.1 eq) was added. The mixture was stirred at rt overnight. The mixture was purified by prep. HPLC (column: Waters X-Bridge, 30×75 mm, 10 um, UV/MS, basic conditions). LC-MS (1): t_{R}=1.17 min; [M+H]⁺: 331.1.

Example 13 to Example 89 were synthesized using the appropriate amine or amine salt (HCl or TFA) derivative and following the procedure described in Example 12. LC-MS data of Example 13 to Example 89 are listed in the table below. The LC-MS conditions used were LC-MS (1).

| Example No | Name | t_{R} | [M + H]⁺ |
|---|---|---|---|
| 13 | 1-(3-Difluoromethoxy-benzyl)-3-spiro[3.3]hept-2-yl-urea | 1.08 | 311.1 |
| 14 | 1-Spiro[3.3]hept-2-yl-3-[3-(2,2,2-trifluoro-ethoxy)-benzyl]- | 1.14 | 343.1 |
| | urea | | |
| 15 | 1-(3-Difluoromethyl-benzyl)-3-spiro[3.3]hept-2-yl-urea | 1.07 | 295.21 |
| 16 | 1-(2-Fluoro-5-trifluoromethyl-benzyl)-3-spiro[3.3]hept-2- | 1.16 | 331.1 |
| | yl-urea | | |
| 17 | 1-(4-Fluoro-3-trifluoromethyl-benzyl)-3-spiro[3.3]hept-2- | 1.16 | 331.0 |
| | yl-urea | | |
| 18 | 1-(3-Fluoro-5-trifluoromethyl-benzyl)-3-spiro[3.3]hept-2- | 1.18 | 331.0 |
| | yl-urea | | |
| 19 | (±)-1-[1-(2-Bromo-5-trifluoromethyl-phenyl)-ethyl]-3- | 1.28 | 405.0 |
| | spiro[3.3]hept-2-yl-urea | | |
| 20 | (±)-1-[1-(2-Methoxy-5-trifluoromethyl-phenyl)-ethyl]-3- | 1.22 | 357.1 |
| | spiro[3.3]hept-2-yl-urea | | |
| 21 | (±)-1-[1-(2-Methoxy-3-trifluoromethyl-phenyl)-ethyl]-3- | 1.22 | 357.1 |
| | spiro[3.3]hept-2-yl-urea | | |
| 22 | 1-(2-Methoxy-3-trifluoromethyl-benzyl)-3-spiro[3.3]hept- | 1.17 | 343.1 |
| | 2-yl-urea | | |
| 23 | (±)-1-Spiro[3.3]hept-2-yl-3-[1-(3-trifluoromethyl-phenyl)- | 1.19 | 327.1 |
| | ethyl]-urea | | |
| 24 | (±)-1-[Cyclopropyl-(3-trifluoromethyl-phenyl)-methyl]-3- | 1.25 | 353.1 |
| | spiro[3.3]hept-2-yl-urea | | |
| 25 | (±)-1-[Cyclopropyl-(3-difluoromethoxy-phenyl)-methyl]-3- | 1.19 | 351.1 |
| | spiro[3.3]hept-2-yl-urea | | |
| 26 | (±)-1-{1-[4-Methyl-3-(2,2,2-trifluoro-ethoxy)-phenyl]- | 1.26 | 371.1 |
| | ethyl}-3-spiro[3.3]hept-2-yl-urea | | |
| 27 | (±)-1-{1-[4-Chloro-3-(2,2,2-trifluoro-ethoxy)-phenyl]- | 1.25 | 391.1 |
| | ethyl}-3-spiro[3.3]hept-2-yl-urea | | |
| 28 | (±)-1-{1-[4-Methoxy-3-(2,2,2-trifluoro-ethoxy)-phenyl]- | 1.16 | 387.2 |
| | ethyl}-3-spiro[3.3]hept-2-yl-urea | | |
| 29 | (±)-1-{1-[2-Methyl-3-(2,2,2-trifluoro-ethoxy)-phenyl]- | 1.23 | 371.2 |
| | ethyl}-3-spiro[3.3]hept-2-yl-urea | | |
| 30 | (±)-1-{1-[2-Chloro-3-(2,2,2-trifluoro-ethoxy)-phenyl]- | 1.22 | 391.1 |
| | ethyl}-3-spiro[3.3]hept-2-yl-urea | | |
| 31 | (±)-1-{1-[2-Methoxy-3-(2,2,2-trifluoro-ethoxy)-phenyl]- | 1.20 | 387.1 |
| | ethyl}-3-spiro[3.3]hept-2-yl-urea | | |
| 32 | (±)-1-{1-[3-Methyl-5-(2,2,2-trifluoro-ethoxy)-phenyl]- | 1.24 | 371.2 |
| | ethyl}-3-spiro[3.3]hept-2-yl-urea | | |
| 33 | (±)-1-{1-[3-Chloro-5-(2,2,2-trifluoro-ethoxy)-phenyl]- | 1.27 | 391.1 |
| | ethyl}-3-spiro[3.3]hept-2-yl-urea | | |
| 34 | (±)-1-(1-(3-(pentafluoro-λ⁶-sulfaneyl)phenyl)ethyl)-3- | 1.23 | 385.1 |
| | (spiro[3.3]heptan-2-yl)urea | | |
| 35 | 1-(3-(pentafluoro-λ⁶-sulfaneyl)benzyl)-3- | 1.19 | 371.0 |
| | (spiro[3.3]heptan-2-yl)urea | | |
| 36 | (±)-1-[1-(3-Methanesulfonyl-phenyl)-ethyl]-3- | 0.92 | 337.1 |
| | spiro[3.3]hept-2-yl-urea | | |
| 37 | 1-Spiro[3.3]hept-2-yl-3-[3-(1-trifluoromethyl-cyclopropyl)- | 1.23 | 353.1 |
| | benzyl]-urea | | |
| 38 | 1-(2-Hydroxy-5-trifluoromethyl-benzyl)-3-spiro[3.3]hept- | 1.20 | 329.1 |
| | 2-yl-urea | | |
| 39 | (±)-1-{1-[2-Chloro-5-(2,2,2-trifluoro-ethoxy)-phenyl]- | 1.25 | 391.1 |
| | ethyl}-3-spiro[3.3]hept-2-yl-urea | | |
| 40 | (±)-1-{1-[2-Methoxy-5-(2,2,2-trifluoro-ethoxy)-phenyl]- | 1.21 | 387.1 |
| | ethyl}-3-spiro[3.3]hept-2-yl-urea | | |
| 41 | 1-Spiro[3.3]hept-2-yl-3-(3-trifluoromethoxy-benzyl)-urea | 1.17 | 329.1 |
| 42 | 1-Spiro[3.3]hept-2-yl-3-(3-trifluoromethanesulfonyl- | 1.14 | 377.0 |
| | benzyl)-urea | | |
| 43 | 1-Spiro[3.3]hept-2-yl-3-(3-trifluoromethoxymethyl- | 1.18 | 343.1 |
| | benzyl)-urea | | |
| 44 | 1-Spiro[3.3]hept-2-yl-3-[3-(3-trifluoromethyl-phenyl)- | 1.11 | 355.1 |
| | oxetan-3-yl]-urea | | |
| 45 | (±)-1-[2-Hydroxy-2-methyl-1-(3-trifluoromethyl-phenyl)- | 1.12 | 371.1 |
| | propyl]-3-spiro[3.3]hept-2-yl-urea | | |
| 46 | 1-(2,2-Difluoro-benzo[1,3]dioxol-4-ylmethyl)-3- | 1.15 | 325.0 |
| | spiro[3.3]hept-2-yl-urea | | |
| 47 | (±)-1-[1-(2,2-Difluoro-benzo[1,3]dioxol-4-yl)-ethyl]-3- | 1.20 | 339.1 |
| | spiro[3.3]hept-2-yl-urea | | |
| 48 | (±)-1-{2-Dimethylamino-1-[6-(2,2,2-trifluoro-ethoxy)- | 0.76 | 401.2 |
| | pyridin-2-yl]-ethyl}-3-spiro[3.3]hept-2-yl-urea | | |
| 49 | 1-Spiro[3.3]hept-2-yl-3-{4-[2-(2,2,2-trifluoro-ethoxy)- | 1.13 | 414.2 |
| | pyridin-4-yl]-tetrahydro-pyran-4-yl}-urea | | |
| 50 | (±)-1-{2-Methoxy-1-[2-(2,2,2-trifluoro-ethoxy)-pyridin-4- | 1.22 | 402.2 |
| | yl]-ethyl}-1-methyl-3-spiro[3.3]hept-2-yl-urea | | |
| 51 | 1-[1-(2-Difluoromethoxy-pyridin-4-yl)-cyclobutyl]-3- | 1.15 | 352.1 |
| | spiro[3.3]hept-2-yl-urea | | |
| 52 | 1-[1-(2-Difluoromethoxy-pyridin-4-yl)-1-methyl-ethyl]-3- | 1.13 | 340.1 |
| | spiro[3.3]hept-2-yl-urea | | |
| 53 | 5-(2,2,2-Trifluoro-ethoxy)-3,4-dihydro-1H- | 1.23 | 370.1 |
| | [2,6]naphthyridine-2-carboxylic acid spiro[3.3]hept-2- | | |
| | ylamide | | |
| 54 | (±)-1-Methoxymethyl-5-(2,2,2-trifluoro-ethoxy)-3,4- | 1.28 | 413.2 |
| | dihydro-1H-isoquinoline-2-carboxylic acid spiro[3.3]hept- | | |
| | 2-ylamide | | |
| 55 | 1-[Cyano-(3-trifluoromethyl-phenyl)-methyl]-3- | 1.17 | 338.1 |
| | spiro[3.3]hept-2-yl-urea | | |
| 56 | 1-Spiro[3.3]hept-2-yl-3-[(1H-[1,2,4]triazol-3-yl)-(3- | 1.01 | 380.1 |
| | trifluoromethyl-phenyl)-methyl]-urea | | |
| 57 | 1-[2,2-Difluoro-1-(3-trifluoromethyl-phenyl)-ethyl]-3- | 1.21 | 363.0 |
| | spiro[3.3]hept-2-yl-urea | | |
| 58 | 1-Spiro[3.3]hept-2-yl-3-[2,2,2-trifluoro-1-(3- | 1.28 | 381.1 |
| | trifluoromethyl-phenyl)-ethyl]-urea | | |
| 59 | 1-Spiro[3.3]hept-2-yl-3-[6-(2,2,2-trifluoro-ethoxy)- | 1.02 | 345.1 |
| | pyrimidin-4-ylmethyl]-urea | | |
| 60 | 1-[2-Methyl-6-(2,2,2-trifluoro-ethoxy)-pyrimidin-4- | 1.06 | 359.1 |
| | ylmethyl]-3-spiro[3.3]hept-2-yl-urea | | |
| 61 | (±)-1-Spiro[3.3]hept-2-yl-3-{1-[6-(2,2,2-trifluoro-ethoxy)- | 1.08 | 359.1 |
| | pyrimidin-4-yl]-ethyl}-urea | | |
| 62 | (±)-2-(3-Spiro[3.3]hept-2-yl-ureido)-2-(3-trifluoromethyl- | 1.01 | 356.1 |
| | phenyl)-acetamide | | |
| 63 | (±)-N-Methyl-2-(3-spiro[3.3]hept-2-yl-ureido)-2-(3- | 1.05 | 370.1 |
| | trifluoromethyl-phenyl)-acetamide | | |
| 64 | (±)-1-[2-Methanesulfonyl-1-(3-trifluoromethoxy-phenyl)- | 1.10 | 421.1 |
| | ethyl]-3-spiro[3.3]hept-2-yl-urea | | |
| 65 | (±)-1-[1-(2,2-Difluoro-benzo[1,3]dioxol-5-yl)-ethyl]-3- | 1.20 | 339.1 |
| | spiro[3.3]hept-2-yl-urea | | |
| 66 | (±)-1-[2-Cyano-1-(3-trifluoromethoxy-phenyl)-ethyl]-3- | 1.16 | 368.1 |
| | spiro[3.3]hept-2-yl-urea | | |
| 67 | (±)-1-[2-Cyano-1-(3-trifluoromethyl-phenyl)-ethyl]-3- | 1.13 | 352.1 |
| | spiro[3.3]hept-2-yl-urea | | |
| 68 | (±)-1-{1-[2-Methyl-6-(2,2,2-trifluoro-ethoxy)-pyrimidin-4- | 1.13 | 373.2 |
| | yl]-ethyl}-3-spiro[3.3]hept-2-yl-urea | | |
| 69 | (±)-1-[2-Methanesulfonyl-1-(3-trifluoromethyl-phenyl)- | 1.07 | 405.1 |
| | ethyl]-3-spiro[3.3]hept-2-yl-urea | | |
| 70 | 1-Spiro[3.3]hept-2-yl-3-[2-(2,2,2-trifluoro-ethoxy)- | 1.01 | 345.1 |
| | pyrimidin-4-ylmethyl]-urea | | |
| 71 | (±)-1-[2-(2-Methoxy-ethoxy)-1-(3-trifluoromethyl-phenyl)- | 1.18 | 401.2 |
| | ethyl]-3-spiro[3.3]hept-2-yl-urea | | |
| 72 | (±)-1-[2-Isopropoxy-1-(3-trifluoromethyl-phenyl)-ethyl]-3- | 1.30 | 385.2 |
| | spiro[3.3]hept-2-yl-urea | | |
| 73 | (±)-1-[3-Ethoxy-1-(3-trifluoromethyl-phenyl)-propyl]-3- | 1.26 | 385.2 |
| | spiro[3.3]hept-2-yl-urea | | |
| 74 | (±)-1-[3-Hydroxy-1-(3-trifluoromethyl-phenyl)-propyl]-3- | 1.06 | 357.1 |
| | spiro[3.3]hept-2-yl-urea | | |
| 75 | (±)-1-[3-Methylsulfanyl-1-(3-trifluoromethyl-phenyl)- | 1.27 | 387.1 |
| | propyl]-3-spiro[3.3]hept-2-yl-urea | | |
| 76 | (±)-1-[2-Hydroxy-1-(3-trifluoromethyl-phenyl)-ethyl]-3- | 1.05 | 343.1 |
| | spiro[3.3]hept-2-yl-urea | | |
| 77 | (±)-1-{3-Ethoxy-1-[2-(2,2,2-trifluoro-ethoxy)-pyridin-4-yl]- | 1.22 | 416.2 |
| | propyl}-3-spiro[3.3]hept-2-yl-urea | | |
| 78 | (±)-1-{2-Methoxy-1-[6-(2,2,2-trifluoro-ethoxy)-pyrimidin-4- | 1.08 | 389.1 |
| | yl]-ethyl}-3-spiro[3.3]hept-2-yl-urea | | |
| 79 | (±)-4-(3-Spiro[3.3]hept-2-yl-ureido)-4-(3-trifluoromethyl- | 1.00 | 384.2 |
| | phenyl)-butyramide | | |
| 80 | (±)-1-Spiro[3.3]hept-2-yl-3-[1-(3-trifluoromethyl-phenyl)- | 1.26 | 353.1 |
| | but-3-enyl]-urea | | |
| 81 | (±)-1-[3-Methanesulfonyl-1-(3-trifluoromethyl-phenyl)- | 1.08 | 419.1 |
| | propyl]-3-spiro[3.3]hept-2-yl-urea | | |
| 82 | (±)-1-{2-Hydroxy-1-[2-methyl-6-(2,2,2-trifluoro-ethoxy)- | 1.02 | 389.1 |
| | pyrimidin-4-yl]-ethyl}-3-spiro[3.3]hept-2-yl-urea | | |
| 83 | (±)-1-{2-Hydroxy-1-[6-(2,2,2-trifluoro-ethoxy)-pyrimidin-4- | 0.97 | 375.1 |
| | yl]-ethyl}-3-spiro[3.3]hept-2-yl-urea | | |
| 84 | (±)-1-[3,3-Difluoro-1-(3-trifluoromethyl-phenyl)-propyl]-3- | 1.22 | 377.1 |
| | spiro[3.3]hept-2-yl-urea | | |
| 85 | (±)-1-{3-Hydroxy-1-[6-(2,2,2-trifluoro-ethoxy)-pyrimidin-4- | 0.98 | 389.1 |
| | yl]-propyl}-3-spiro[3.3]hept-2-yl-urea | | |
| 86 | (±)-1-[1-(3-Methanesulfonyl-phenyl)-2-methoxy-ethyl]-3- | 0.91 | 367.1 |
| | spiro[3.3]hept-2-yl-urea | | |
| 87 | (±)-1-{2-Hydroxy-1-[3-(2,2,2-trifluoro-ethoxy)-phenyl]- | 1.05 | 373.1 |
| | ethyl}-3-spiro[3.3]hept-2-yl-urea | | |
| 88 | (±)-1-{3-Hydroxy-1-[3-(2,2,2-trifluoro-ethoxy)-phenyl]- | 1.07 | 387.1 |
| | propyl}-3-spiro[3.3]hept-2-yl-urea | | |
| 89 | 1-[(±)-Cyano-(3-trifluoromethyl-phenyl)-methyl]-3- | 1.17 | 338.1 |
| | spiro[3.3]hept-2-yl-urea | | |

### Example 90: (±)-1-Spiro[3.3]hept-2-yl-3-{1-[3-(2,2,2-trifluoro-ethoxy)-phenyl]-ethyl}-urea

To a solution of (±)-1-(3-(2,2,2-trifluoroethoxy)phenyl)ethan-1-amine trifluoroacetate (17 mg, 0.05 mmol, 1.0 eq) and NEt₃ (36 μL, 0.25 mmol, 5.0 eq) in MeCN (2 mL), CDI (10 mg, 0.06 mmol, 1.2 eq) was added. The reaction mixture was stirred at rt for 1 hour. Then spiro[3.3]heptan-2-amine hydrochloride (8 mg, 0.055 mmol, 1.1 eq) was added and the mixture was stirred at rt overnight. The mixture was purified by prep. HPLC (column: Waters X-Bridge, 30×75 mm, 10 um, UV/MS, basic conditions). LC-MS (1): t_{R}=1.18 min; [M+H]⁺: 357.1.

Example 91 to Example 109 were synthesized using the appropriate amine or amine salt (HCl or TFA) derivative and following the procedure described in Example 90. LC-MS data of Example 91 to Example 109 are listed in the table below. The LC-MS conditions used were LC-MS (1).

| Example No | Name | t_{R} | [M + H]⁺ |
|---|---|---|---|
| 91 | (±)-1-{2-Methoxy-1-[3-(2,2,2-trifluoro-ethoxy)-phenyl]- | 1.16 | 387.1 |
| | ethyl}-3-spiro[3.3]hept-2-yl-urea | | |
| 92 | (±)-1-{2-Methoxy-1-[2-(2,2,2-trifluoro-ethoxy)-pyridin-4- | 1.15 | 388.2 |
| | yl]-ethyl}-3-spiro[3.3]hept-2-yl-urea | | |
| 93 | (±)-1-Spiro[3.3]hept-2-yl-3-{1-[2-(2,2,2-trifluoro-ethoxy)- | 1.15 | 358.1 |
| | pyridin-4-yl]-ethyl}-urea | | |
| 94 | (±)-1-{2-Dimethylamino-1-[2-(2,2,2-trifluoro-ethoxy)- | 0.73 | 401.2 |
| | pyridin-4-yl]-ethyl}-3-spiro[3.3]hept-2-yl-urea | | |
| 95 | (±)-1-{1-[3-Fluoro-5-(2,2,2-trifluoro-ethoxy)-phenyl]- | 1.21 | 375.1 |
| | ethyl}-3-spiro[3.3]hept-2-yl-urea | | |
| 96 | 1-Spiro[3.3]hept-2-yl-3-(2-trifluoromethoxy-pyridin-4- | 1.06 | 330.1 |
| | ylmethyl)-urea | | |
| 97 | (±)-1-Spiro[3.3]hept-2-yl-3-[1-(2-trifluoromethoxy-pyridin- | 1.11 | 344.1 |
| | 4-yl)-ethyl]-urea | | |
| 98 | (±)-1-{1-[2-Fluoro-3-(2,2,2-trifluoro-ethoxy)-phenyl]- | 1.19 | 375.1 |
| | ethyl}-3-spiro[3.3]hept-2-yl-urea | | |
| 99 | (±)-1-{1-[2-Fluoro-5-(2,2,2-trifluoro-ethoxy)-phenyl]- | 1.20 | 375.1 |
| | ethyl}-3-spiro[3.3]hept-2-yl-urea | | |
| 100 | (±)-1-[1-(2-Difluoromethoxy-pyridin-4-yl)-ethyl]-3- | 1.08 | 326.1 |
| | spiro[3.3]hept-2-yl-urea | | |
| 101 | 1-(Spiro[3.3]heptan-2-yl)-3-(1-(2-((1,1,1-trifluoropropan- | 1.22 | 372.1 |
| | 2-yl)oxy)pyridin-4-yl)ethyl)urea | | |
| 102 | 1-(Spiro[3.3]heptan-2-yl)-3-(1-(3-((1,1,1-trifluoropropan- | 1.23 | 371.1 |
| | 2-yl)oxy)phenyl)ethyl)urea | | |
| 103 | 1-Spiro[3.3]hept-2-yl-3-[2-(2,2,2-trifluoro-ethoxy)-pyridin- | 1.11 | 344.1 |
| | 4-ylmethyl]-urea | | |
| 104 | (±)-1-Spiro[3.3]hept-2-yl-3-[6-(2,2,2-trifluoro-1- | 1.16 | 388.2 |
| | methoxymethyl-ethoxy)-pyridin-2-ylmethyl]-urea | | |
| 105 | (±)-1-[6-(1-Dimethylaminomethyl-2,2,2-trifluoro-ethoxy)- | 0.68 | 401.2 |
| | pyridin-2-ylmethyl]-3-spiro[3.3]hept-2-yl-urea | | |
| 106 | (±)-1-[2-(1-Dimethylaminomethyl-2,2,2-trifluoro-ethoxy)- | 0.66 | 401.2 |
| | pyridin-4-ylmethyl]-3-spiro[3.3]hept-2-yl-urea | | |
| 107 | (±)-1-Spiro[3.3]hept-2-yl-3-[2-(2,2,2-trifluoro-1- | 1.14 | 388.1 |
| | methoxymethyl-ethoxy)-pyridin-4-ylmethyl]-urea | | |
| 108 | 1-[5-Methyl-6-(2,2,2-trifluoro-ethoxy)-pyridin-2-ylmethyl]- | 1.23 | 358.1 |
| | 3-spiro[3.3]hept-2-yl-urea | | |
| 109 | 1-[4-Methyl-3-(2,2,2-trifluoro-ethoxy)-benzyl]-3- | 1.22 | 357.1 |
| | spiro[3.3]hept-2-yl-urea | | |

### Example 110: (±)-1-[3-Fluoro-5-(2,2,2-trifluoro-1-methyl-ethoxy)-benzyl]-3-spiro[3.3]hept-2-yl-urea

To a solution of (±)-(3-fluoro-5-((1,1,1-trifluoropropan-2-yl)oxy)phenyl)methanamine (49 mg, 0.2 mmol, 1.00 eq) in MeCN (1 mL), DIPEA (0.12 mL, 0.7 mmol, 3.50 eq) and CDI (34 mg, 0.21 mmol, 1.05 eq) were added in sequence. The resulting mixture was stirred at 50° C. for 1.5 h. 2-Aminospiro[3.3]heptane hydrochloride (30 mg, 0.2 mmol, 1 eq) was added and the reaction mixture was stirred at 80° C. overnight. The mixture was purified by prep. HPLC (column: Waters X-Bridge, 30×75 mm, 10 um, UV/MS, acidic conditions). LC-MS (1): t_{R}=1.22 min; [M+H]⁺: 375.1.

Example 111 to Example 119 were synthesized using the appropriate amine or amine salt (HCl or TFA) derivative and following the procedure described in Example 110. LC-MS data of Example 111 to Example 119 are listed in the table below. The LC-MS conditions used were LC-MS (1).

| Example No | Name | t_{R} | [M + H]⁺ |
|---|---|---|---|
| 111 | (±)-1-Spiro[3.3]hept-2-yl-3-{1-[3-(2,2,2-trifluoro-1-methyl- | 1.24 | 383.1 |
| | ethoxy)-phenyl]-cyclopropyl}-urea | | |
| 112 | 1-Spiro[3.3]hept-2-yl-3-[4-(2,2,2-trifluoro-ethoxy)- | 1.01 | 345.1 |
| | pyrimidin-2-ylmethyl]-urea | | |
| 113 | (±)-1-{1-[4-Fluoro-3-(2,2,2-trifluoro-ethoxy)-phenyl]- | 1.19 | 375.1 |
| | ethyl}-3-spiro[3.3]hept-2-yl-urea | | |
| 114 | 1-[4-Fluoro-3-(2,2,2-trifluoro-ethoxy)-benzyl]-3- | 1.15 | 361.1 |
| | spiro[3.3]hept-2-yl-urea | | |
| 115 | 1-{2-[Methyl-(2,2,2-trifluoro-ethyl)-amino]-pyridin-4- | 0.79 | 357.1 |
| | ylmethyl}-3-spiro[3.3]hept-2-yl-urea | | |
| 116 | 1-Spiro[3.3]hept-2-yl-3-[2-(2,2,2-trifluoro-ethylamino)- | 0.64 | 343.1 |
| | pyridin-4-ylmethyl]-urea | | |
| 117 | (±)-1-Spiro[3.3]hept-2-yl-3-[4-(2,2,2-trifluoro-1-methyl- | 0.76 | 358.1 |
| | ethoxy)-pyridin-2-ylmethyl]-urea | | |
| 118 | (±)-1-Spiro[3.3]hept-2-yl-3-[2-(2,2,2-trifluoro-1-methyl- | 1.07 | 359.1 |
| | ethoxy)-pyrimidin-4-ylmethyl]-urea | | |
| 119 | 1-Spiro[3.3]hept-2-yl-3-[1-(3-trifluoromethoxy-phenyl)- | 1.22 | 343.1 |
| | ethyl]-urea | | |

### Example 120: (±)-1-Spiro[2.3]hex-5-yl-3-[6-(2,2,2-trifluoro-1-methyl-ethoxy)-pyrimidin-4-ylmethyl]-urea

To a solution of spiro[2.3]hex-5-ylamine hydrochloride (10 mg, 0.08 mmol, 1 eq) and NEt₃ (32 μL, 0.23 mmol, 3 eq) in MeCN (2 mL), CDI (15 mg, 0.09 mmol, 1.2 eq) was added. The reaction mixture was stirred at rt for 1 hour. (±)-(6-((1,1,1-Trifluoropropan-2-yl)oxy)pyrimidin-4-yl)methanamine (17 mg, 0.08 mmol, 1 eq) was added and the mixture was stirred at rt overnight. The mixture was purified by prep. HPLC (column: Waters X-Bridge, 30×75 mm, 10 um, UV/MS, basic conditions). LC-MS (1): t_{R}=1.01 min; [M+H]⁺: 345.1.

Example 121 to Example 123 were synthesized using the appropriate amine or amine salt (HCl or TFA) derivative and following the procedure described in Example 120. LC-MS data of Example 121 to Example 123 are listed in the table below. The LC-MS conditions used were LC-MS (1).

| Example No | Name | t_{R} | [M + H]⁺ |
|---|---|---|---|
| 121 | (±)-1-[1-(2,2-Difluoro-benzo[1,3]dioxol-4-yl)-ethyl]-3- | 1.13 | 325.1 |
| | spiro[2.3]hex-5-yl-urea | | |
| 122 | (±)-1-{2-Hydroxy-1-[6-(2,2,2-trifluoro-ethoxy)-pyrimidin-4- | 0.89 | 361.1 |
| | yl]-ethyl}-3-spiro[2.3]hex-5-yl-urea | | |
| 123 | (±)-1-{2-Hydroxy-1-[2-methyl-6-(2,2,2-trifluoro-ethoxy)- | 0.94 | 375.1 |
| | pyrimidin-4-yl]-ethyl}-3-spiro[2.3]hex-5-yl-urea | | |

### Example 124: (±)-1-[1-(3-Difluoromethoxy-phenyl)-ethyl]-3-spiro[2.3]hex-5-yl-urea

To a solution of (±)-1-(3-(difluoromethoxy)phenyl)ethan-1-amine hydrochloride (37 mg, 0.1 mmol, 1 eq) in MeCN, DIPEA (51 μL, 0.3 mmol, 3 eq) and CDI (32 mg, 0.2 mmol, 2 eq) were added. The mixture was stirred at rt for 1 hour. A solution of spiro[2.3]hex-5-ylamine hydrochloride (0.1 mmol, 1 eq) in MeCN (0.4 mL) and H₂O (0.1 mL) was added. The reaction mixture was stirred at rt for 1 hour. The mixture was purified by prep. HPLC (column: Waters X-Bridge, 30×75 mm, 10 um, UV/MS, basic conditions). LC-MS (1): t_{R}=1.05 min; [M+H]⁺: 311.1.

### Example 125: 1-(2-Methoxy-5-trifluoromethyl-benzyl)-3-spiro[2.3]hex-5-yl-urea

To a solution of spiro[2.3]hex-5-ylamine hydrochloride (14 mg, 0.10 mmol, 1.0 eq) in MeCN (0.5 mL), CDI (24 mg, 0.15 mmol, 1.5 eq) and DIPEA (34 μL, 0.20 mmol, 2.0 eq) were added. The mixture was stirred at 60° C. for 1 hour. A solution of (2-methoxy-5-(trifluoromethyl)phenyl)methanamine hydrochloride (36 mg, 0.15 mmol, 1.5 eq) and DIPEA (34 μL, 0.2 mmol, 2 eq) in MeCN (0.4 mL) and H₂O (0.1 mL) was added. The reaction mixture was further stirred at 60° C. overnight. The mixture was allowed to cool to rt and purified by prep. HPLC (column: Waters X-Bridge, 30×75 mm, 10 um, UV/MS, acidic conditions). LC-MS (1): t_{R}=1.10 min; [M+H]⁺: 329.1.

Example 126 to Example 142 were synthesized using the appropriate amine or amine salt (HCl or TFA) derivative and following the procedure described in Example 125. LC-MS data of Example 126 to Example 142 are listed in the table below. The LC-MS conditions used were LC-MS (1).

| Example No | Name | t_{R} | [M + H]⁺ |
|---|---|---|---|
| 126 | 1-Spiro[2.3]hex-5-yl-3-[4-(2,2,2-trifluoro-ethoxy)- | 0.93 | 331.1 |
| | pyrimidin-2-ylmethyl]-urea | | |
| 127 | (±)-1-(1-(3-(pentafluoro-λ⁶-sulfaneyl)phenyl)ethyl)-3- | 1.17 | 371.1 |
| | (spiro[2.3]hexan-5-yl)urea | | |
| 128 | 1-(2-Methoxy-3-trifluoromethyl-benzyl)-3-spiro[2.3]hex-5- | 1.09 | 329.1 |
| | yl-urea | | |
| 129 | (±)-1-{1-[2-Fluoro-5-(2,2,2-trifluoro-ethoxy)-phenyl]- | 1.14 | 361.1 |
| | ethyl}-3-spiro[2.3]hex-5-yl-urea | | |
| 130 | 1-(3-(pentafluoro-λ⁶-sulfaneyl)benzyl)-3-(spiro[2.3]hexan- | 1.12 | 357.0 |
| | 5-yl)urea | | |
| 131 | (±)-1-{1-[2-Fluoro-3-(2,2,2-trifluoro-ethoxy)-phenyl]- | 1.12 | 361.1 |
| | ethyl}-3-spiro[2.3]hex-5-yl-urea | | |
| 132 | (±)-1-{1-[3-Fluoro-5-(2,2,2-trifluoro-ethoxy)-phenyl]- | 1.15 | 361.1 |
| | ethyl}-3-spiro[2.3]hex-5-yl-urea | | |
| 133 | (±)-1-[1-(2-Methoxy-3-trifluoromethyl-phenyl)-ethyl]-3- | 1.14 | 343.1 |
| | spiro[2.3]hex-5-yl-urea | | |
| 134 | 1-(2-Fluoro-3-trifluoromethoxy-benzyl)-3-spiro[2.3]hex-5- | 1.12 | 333.0 |
| | yl-urea | | |
| 135 | (±)-1-Spiro[2.3]hex-5-yl-3-{1-[3-(2,2,2-trifluoro-1-methyl- | 1.17 | 369.1 |
| | ethoxy)-phenyl]-cyclopropyl}-urea | | |
| 136 | 1-[1-(3-Difluoromethoxy-phenyl)-cyclopropyl]-3- | 1.06 | 323.1 |
| | spiro[2.3]hex-5-yl-urea | | |
| 137 | (±)-1-[1-(2-Methoxy-5-trifluoromethyl-phenyl)-ethyl]-3- | 1.15 | 343.1 |
| | spiro[2.3]hex-5-yl-urea | | |
| 138 | (±)-1-[3-Hydroxy-1-(3-trifluoromethoxy-phenyl)-propyl]-3- | 1.02 | 359.1 |
| | spiro[2.3]hex-5-yl-urea | | |
| 139 | (±)-1-[1-(2-Difluoromethoxy-pyridin-4-yl)-ethyl]-3- | 0.99 | 312.1 |
| | spiro[2.3]hex-5-yl-urea | | |
| 140 | (±)-1-Spiro[2.3]hex-5-yl-3-[1-(3-trifluoromethyl-phenyl)- | 1.12 | 313.1 |
| | ethyl]-urea | | |
| 141 | (±)-1-Spiro[2.3]hex-5-yl-3-[1-(3-trifluoromethoxy-phenyl)- | 1.15 | 329.1 |
| | ethyl]-urea | | |
| 142 | 1-(3-Fluoro-5-trifluoromethyl-benzyl)-3-spiro[2.3]hex-5-yl- | 1.10 | 317.1 |
| | urea | | |

### Example 143: (±)-1-[2-Hydroxy-1-(3-trifluoromethyl-phenyl)-ethyl]-3-spiro[2.3]hex-5-yl-urea

To a solution of spiro[2.3]hex-5-ylamine hydrochloride (21 mg, 0.15 mmol, 1.0 eq) in MeCN (0.8 mL), CDI (37 mg, 0.23 mmol, 1.5 eq) and DIPEA (92 μL, 0.53 mmol, 3.5 eq) were added. The mixture was stirred at 50° C. for 1 hour. 2-Amino-2-(3-trifluoromethyl-phenyl)-ethanol (31 mg, 0.15 mmol, 1.0 eq) was added. The reaction mixture was further stirred at 80° C. overnight. The mixture was allowed to cool to rt and purified by prep. HPLC (column: Waters X-Bridge, 30×75 mm, 10 um, UV/MS, basic conditions). LC-MS (1): t_{R}=0.97 min; [M+H]⁺: 329.1.

Example 144 to Example 152 were synthesized using the appropriate amine or amine salt (HCl or TFA) derivative and following the procedure described in Example 143. LC-MS data of Example 144 to Example 152 are listed in the table below. The LC-MS conditions used were LC-MS (1).

| Example No | Name | t_{R} | [M + H]⁺ |
|---|---|---|---|
| 144 | (±)-1-Spiro[2.3]hex-5-yl-3-{1-[6-(2,2,2-trifluoro-ethoxy)- | 0.99 | 345.1 |
| | pyrimidin-4-yl]-ethyl}-urea | | |
| 145 | (±)-1-{1-[2-Methyl-6-(2,2,2-trifluoro-ethoxy)-pyrimidin-4- | 1.05 | 359.1 |
| | yl]-ethyl}-3-spiro[2.3]hex-5-yl-urea | | |
| 146 | (±)-1-[2,2-Difluoro-1-(3-trifluoromethyl-phenyl)-ethyl]-3- | 1.14 | 349.1 |
| | spiro[2.3]hex-5-yl-urea | | |
| 147 | (±)-1-[2-Methoxy-1-(3-trifluoromethyl-phenyl)-ethyl]-3- | 1.10 | 343.1 |
| | spiro[2.3]hex-5-yl-urea | | |
| 148 | 1-(4-Fluoro-3-trifluoromethyl-benzyl)-3-spiro[2.3]hex-5-yl- | 1.09 | 317.1 |
| | urea | | |
| 149 | 1-(2-Fluoro-3-trifluoromethyl-benzyl)-3-spiro[2.3]hex-5-yl- | 1.09 | 317.1 |
| | urea | | |
| 150 | 1-(2-Chloro-3-trifluoromethyl-benzyl)-3-spiro[2.3]hex-5- | 1.13 | 333.0 |
| | yl-urea | | |
| 151 | 1-(2-Fluoro-5-trifluoromethyl-benzyl)-3-spiro[2.3]hex-5-yl- | 1.09 | 317.1 |
| | urea | | |
| 152 | (±)-1-{1-[5-Methoxy-6-(2,2,2-trifluoro-ethoxy)-pyrimidin-4- | 1.06 | 375.1 |
| | yl]-ethyl}-3-spiro[2.3]hex-5-yl-urea | | |

### Example 153: 1-Spiro[2.3]hex-5-yl-3-(2-trifluoromethoxy-pyridin-4-ylmethyl)-urea

To a solution of (2-(trifluoromethoxy)pyridin-4-yl)methanamine hydrochloride (23 mg, 0.07 mmol, 1.0 eq) in MeCN (0.4 mL), DIPEA (43 μL, 0.25 mmol, 3.5 eq) and CDI (12 mg, 0.07 mmol, 1.05 eq) were added. The resulting mixture was stirred at 50° C. for 3 h. Spiro[2.3]hexan-5-amine hydrochloride (9 mg, 0.07 mmol, 1.0 eq) was added and the reaction mixture was stirred 80° C. overnight. The mixture was allowed to cool to rt and purified by prep. HPLC (column: Waters X-Bridge, 30×75 mm, 10 um, UV/MS, acidic conditions). LC-MS (1): t_{R}=0.98 min; [M+H]⁺: 316.0.

Example 154 to Example 158 were synthesized using the appropriate amine or amine salt (HCl or TFA) derivative and following the procedure described in Example 153. LC-MS data of Example 154 to Example 158 are listed in the table below. The LC-MS conditions used were LC-MS (1).

| Example No | Name | t_{R} | [M + H]⁺ |
|---|---|---|---|
| 154 | (±)-1-{2-Methoxy-1-[2-(2,2,2-trifluoro-ethoxy)-pyridin-4- | 1.07 | 374.1 |
| | yl]-ethyl}-3-spiro[2.3]hex-5-yl-urea | | |
| 155 | 1-(3-Difluoromethoxy-benzyl)-3-spiro[2.3]hex-5-yl-urea | 1.00 | 297.1 |
| 156 | 1-Spiro[2.3]hex-5-yl-3-(3-trifluoromethoxy-benzyl)-urea | 1.10 | 315.0 |
| 157 | 1-Spiro[2.3]hex-5-yl-3-[3-(2,2,2-trifluoro-ethoxy)-benzyl]- | 1.07 | 329.1 |
| | urea | | |
| 158 | 1-(3-Difluoromethyl-benzyl)-3-spiro[2.3]hex-5-yl-urea | 0.98 | 281.1 |

### Example 159: 1-Spiro[2.3]hex-5-yl-3-[2-(2,2,2-trifluoro-ethoxy)-pyridin-4-ylmethyl]-urea

To a solution of spiro[2.3]hex-5-ylamine hydrochloride (136 mg, 0.97 mmol, 1.1 eq) in THE (11 mL), NEt₃ (0.50 mL, 3.54 mmol, 4.0 eq) and 4-nitrophenyl ((2-(2,2,2-trifluoroethoxy)pyridin-4-yl)methyl)carbamate (329 mg, 0.89 mmol, 1 eq) were added. The resulting mixture was stirred at rt overnight. The mixture was concentrated under reduced pressure. The residue was purified by prep. HPLC (column: Waters X-Bridge, 30×75 mm, 10 um, UV/MS, basic conditions). LC-MS (1): t_{R}=1.03 min; [M+H]⁺: 330.0.

### Example 160: 1-(6,6-Difluoro-spiro[3.3]hept-2-yl)-3-[2-(2,2,2-trifluoro-ethoxy)-pyridin-4-ylmethyl]-urea

The product was synthesized using 6,6-difluorospiro[3.3]heptan-2-amine hydrochloride and following the procedure described in Example 159. LC-MS (1): t_{R}=1.04 min; [M+H]⁺: 380.1.

### Example 161: (±)-1-(6,6-Difluoro-spiro[3.3]hept-2-yl)-3-[1-(3-trifluoromethyl-phenyl)-ethyl]-urea

The product was synthesized using 6,6-difluorospiro[3.3]heptan-2-amine hydrochloride and following the procedure described in Example 140. LC-MS (1): t_{R}=1.12 min; [M+H]⁺: 363.1.

### Example 162: (±)-1-Spiro[3.3]hept-2-yl-3-[(1H-[1,2,3]triazol-4-yl)-(3-trifluoromethyl-phenyl)-methyl]-urea

### Step 1: (±)-1-(spiro[3.3]heptan-2-yl)-3-(1-(3-(trifluoromethyl)phenyl)prop-2-yn-1-yl)urea

To a solution of (±)-1-(3-(trifluoromethyl)phenyl)prop-2-yn-1-amine hydrochloride (21 mg, 0.09 mmol, 1 eq) in MeCN (0.6 mL), NEt₃ (38 μL, 0.27 mmol, 3 eq) and 2-isocyanatospiro[3.3]heptane (15 mg, 0.11 mmol, 1.2 eq) were added. The resulting mixture was stirred at rt overnight. A few drops of H₂O were added and the resulting suspension was filtered. The solids were rinsed with H₂O and dried under HV. LC-MS (3): t_{R}=1.08 min; [M+H]⁺: 337.31.

### Step 2: (±)-1-((1-benzyl-1H-1,2,3-triazol-4-yl)(3-(trifluoromethyl)phenyl)methyl)-3-(spiro[3.3]heptan-2-yl) urea

A solution of (±)-1-(spiro[3.3]heptan-2-yl)-3-(1-(3-(trifluoromethyl)phenyl)prop-2-yn-1-yl)urea (214 mg, 0.61 mmol, 1.0 eq) in DMF (24 mL) (Pump A) and a solution of benzylazide (100 mg, 0.73 mmol, 1.2 eq) and L(+)-ascorbic acid sodium salt (12 mg, 0.06 mmol, 0.1 eq) in DMF (19.8 mL) and water (4.2 mL) (Pump B) were prepared. Using a Vapourtec system, the two solutions were injected in 1:1 ratio in a copper coil reactor. Following parameters were used: injection volume 2 mL A and 2 mL B, temperature 140° C., pressure 13-14 bar, flow total 0.25 mL/min, residence time 40 min. The collected mixture was diluted with water and EtOAc. The layers were separated and the aq. layer was extracted with EtOAc (2×). The comb. org. layers were washed with sat. aq. NaCl soln., dried over MgSO₄, filtered and concentrated in vacuo. The residue was purified by prep. HPLC (column: Waters X-Bridge, 30×75 mm, 10 um, UV/MS, basic conditions). LC-MS (2): t_{R}=1.04 min; [M+H]⁺: 470.24.

### Step 3: (±)-1-Spiro[3.3]hept-2-yl-3-[(1H-[1,2,3]triazol-4-yl)-(3-trifluoromethyl-phenyl)-methyl]-urea

A solution of (±)-1-((1-benzyl-1H-1,2,3-triazol-4-yl)(3-(trifluoromethyl)phenyl)methyl)-3-(spiro[3.3]heptan-2-yl)urea (148 mg, 0.32 mmol, 1 eq) in EtOH (40 mL) was circulated in an HCube-Pro using a 20% Pd(OH)₂/C catalyst cartridge (7 cm) at 70° C., 1 bar, and with a flow of 0.7 mL/min under 100% H₂ mode. The solvent was then removed in vacuo. The residue was purified by prep. HPLC (column: Waters X-Bridge, 30×75 mm, 10 um, UV/MS, basic conditions) to give a white solid. LC-MS (1): t_{R}=1.05 min; [M+H]⁺: 380.1.

### Example 163: (±)-1-[(1-Methyl-1H-tetrazol-5-yl)-(3-trifluoromethyl-phenyl)-methyl]-3-spiro[3.3]hept-2-yl-urea and Example 164: (±)-1-[(2-Methyl-2H-tetrazol-5-yl)-(3-trifluoromethyl-phenyl)-methyl]-3-spiro[3.3]hept-2-yl-urea

### Step 1: (±)-1-((2H-tetrazol-5-yl)(3-(trifluoromethyl)phenyl)methyl)-3-(spiro[3.3]heptan-2-yl)urea

A solution of 1-[cyano-(3-trifluoromethyl-phenyl)-methyl]-3-spiro[3.3]hept-2-yl-urea (180 mg, 0.49 mmol, 1.0 eq) in DMF (8 mL) (Pump A) and a solution of ZnBr₂ (338 mg, 1.47 mmol, 3 eq) and sodium azide (129 mg, 1.96 mmol, 4 eq) in DMF (12 mL) and water (4 mL) (Pump B) were prepared. Using a Vapourtec system, the two solutions were injected in 1:1 ratio in a stainless steel coil reactor. Following parameters were used: injection volume 2 mL A and 2 mL B, temperature 150° C., pressure 16 bar, flow total 0.25 mL/min, residence time 40 min. The collected mixture was diluted with water, 1M aq. HCl soln. and EtOAc. The layers were separated and the aq. layer was extracted with EtOAc (3×). The comb. org. layers were washed with sat. aq. NaCl soln., dried over MgSO₄, filtered and concentrated in vacuo. The residue was purified by prep. HPLC (column: Waters X-Bridge, 30×75 mm, 10 um, UV/MS, acidic conditions). LC-MS (2): t_{R}=0.91 min; [M+H]⁺: 381.22.

### Step 2: (±)-1-[(1-Methyl-1H-tetrazol-5-yl)-(3-trifluoromethyl-phenyl)-methyl]-3-spiro[3.3]hept-2-yl-urea and (±)-1-[(2-Methyl-2H-tetrazol-5-yl)-(3-trifluoromethyl-phenyl)-methyl]-3-spiro[3.3]hept-2-yl-urea

To a solution of (±)-1-((2H-tetrazol-5-yl)(3-(trifluoromethyl)phenyl)methyl)-3-(spiro[3.3]heptan-2-yl)urea (55 mg, 0.15 mmol, 1.0 eq) in MeCN (1.5 mL), iodomethane (14 μL, 0.22 mmol, 1.5 eq) and NEt₃ (61 μL, 0.43 mmol, 3.0 eq) were added. The reaction mixture was stirred at 40° C. for 2 hours. Iodomethane (27 μL, 0.43 mmol, 3.0 eq) was added and the reaction mixture was heated at 40° C. overnight. The reaction was purified by prep. HPLC (column: Waters X-Bridge, 30×75 mm, 10 um, UV/MS, acidic conditions) to give Example 163 (LC-MS (1): t_{R}=1.12 min; [M+H]⁺: 395.1) and Example 164 (LC-MS (1): t_{R}=1.14 min; [M+H]⁺: 395.1).

### Example 165: (±)-1-[(3-Methyl-3H-[1,2,3]triazol-4-yl)-(3-trifluoromethyl-phenyl)-methyl]-3-spiro[3.3] hept-2-yl-urea

To an ice-cooled solution of (±)-1-spiro[3.3]hept-2-yl-3-[(1H-[1,2,3]triazol-4-yl)-(3-trifluoromethyl-phenyl)-methyl]-urea (15 mg, 0.04 mmol, 1 eq) in DMF (0.5 mL), cesium carbonate (13 mg, 0.04 mmol, 1 eq) and iodomethane (4 μL, 0.06 mmol, 1.5 eq) were added. The mixture was stirred at 0° C. for 30 min and further at rt overnight. The reaction mixture was partitioned between H₂O (10 mL) and EtOAc (10 mL). The aq. phase was extracted with EtOAc (10 mL). The comb. org. phases were dried over Na₂SO₄, filtered and evaporated. The residue was purified by prep. HPLC (column: Waters X-Bridge, 30×75 mm, 10 um, UV/MS, basic conditions) to give a white solid. LC-MS (1): t_{R}=1.15 min; [M+H]⁺: 394.1.

A second fraction containing (±)-1-((1-methyl-1H-1,2,3-triazol-4-yl)(3-(trifluoromethyl)phenyl)methyl)-3-(spiro[3.3]heptan-2-yl)urea was also isolated.

### Example 166: (±)-1-[1-(2-Difluoromethoxy-pyridin-4-yl)-ethyl]-3-(6-iodo-spiro[3.3]hept-2-yl)-urea

### Step 1: (±)-1-(1-(2-(difluoromethoxy)pyridin-4-yl)ethyl)-3-(6-hydroxyspiro[3.3]heptan-2-yl)urea

To a solution under N₂ of 6-aminospiro[3.3]heptan-2-ol hydrochloride (135 mg, 0.80 mmol, 1.2 eq) in MeCN (0.8 mL), DIPEA (0.23 mL, 1.34 mmol, 2.0 eq) and CDI (130 mg, 0.80 mmol, 1.2 eq) were added in sequence. The resulting mixture was stirred at 50° C. for 4 h. (±)-1-(2-(Difluoromethoxy)pyridin-4-yl)ethan-1-amine hydrochloride (150 mg, 0.67 mmol, 1 eq) was added and the reaction mixture was stirred at 80° C. overnight. The reaction mixture was allowed to cool to rt and partitioned between water and EtOAc. The layers were separated. The aq. phase was extracted with EtOAc. The comb. org. phases were washed with sat. aq. NaCl soln., dried over MgSO₄, and concentrated in vacuo. The residue was purified by prep. HPLC (column: Waters X-Bridge, 30×75 mm, 10 um, UV/MS, basic conditions) to afford a light yellow resin. LC-MS (2): t_{R}=0.71 min; [M+H]⁺: 342.24.

### Step 2: (±)-6-(3-(1-(2-(difluoromethoxy)pyridin-4-yl)ethyl) ureido)spiro[3.3]heptan-2-yl methanesulfonate

To an ice-cooled suspension of (±)-1-(1-(2-(difluoromethoxy)pyridin-4-yl)ethyl)-3-(6-hydroxyspiro[3.3]heptan-2-yl)urea (80 mg, 0.23 mmol, 1.0 eq) and NEt₃ (64 μL, 0.47 mmol, 2.0 eq) in DCM (2 mL), a solution of methanesulfonyl chloride (27 μL, 0.35 mmol, 1.5 eq) in DCM (0.4 mL) was added dropwise. The resulting clear solution was stirred at at 0° C. for 45 min. The reaction was partitioned between DCM and water. The aqueous phase was extracted once with DCM and the comb. org. phases were dried over MgSO₄, filtered and concentrated under reduced pressure to afford the crude material as an off-white semi-solid. The product was used without further purification. LC-MS (2): t_{R}=0.83 min; [M+H]⁺: 420.16.

### Step 3: (±)-1-[1-(2-Difluoromethoxy-pyridin-4-yl)-ethyl]-3-(6-iodo-spiro[3.3]hept-2-yl)-urea

To a solution of (±)-6-(3-(1-(2-(difluoromethoxy)pyridin-4-yl)ethyl)ureido)spiro[3.3]heptan-2-yl methanesulfonate (98 mg, 0.23 mmol, 1 eq) in 2-butanone (2.1 mL), sodium iodide (106 mg, 0.70 mmol, 3 eq) was added. The mixture was stirred at 80° C. for 3 h. The reaction mixture was allowed to cool to rt and sodium iodide (106 mg, 0.70 mmol, 3 eq) was added. The reaction mixture was further stirred at 80° C. overnight. The mixture was cooled down to rt and partitioned between EtOAc and water. The layers were separated and the aq. phase was extracted twice with EtOAc. The comb. org. phases were washed with sat. aq. NaCl soln., dried over MgSO₄, filtered and concentrated under reduced pressure. The residue was purified by Combiflash (column: 4 g, flow: 15 mL/min, heptane/EtOAc 3:7). LC-MS (1): t_{R}=1.14 min; [M+H]⁺: 452.0.

### Example 167: 1-[(R)-1-(2-Difluoromethoxy-pyridin-4-yl)-ethyl]-3-spiro[3.3]hept-2-yl-urea and Example 168: 1-[(S)-1-(2-Difluoromethoxy-pyridin-4-yl)-ethyl]-3-spiro[3.3]hept-2-yl-urea

The racemate (±)-1-[1-(2-difluoromethoxy-pyridin-4-yl)-ethyl]-3-spiro[3.3]hept-2-yl-urea was separated by the preparative chiral SFC 2 method to give Example 167 (first eluting, t_{R}=1.9 min) and Example 168 (second eluting, t_{R}=2.7 min). Example 167 (LC-MS (1): t_{R}=1.08 min; [M+H]⁺: 326.1) and Example 168 (LC-MS (1): t_{R}=1.08 min; [M+H]⁺: 326.1).

### Example 169: 1-Spiro[3.3]hept-2-yl-3-{1-[3-(2,2,2-trifluoro-1-methyl-ethoxy)-phenyl]-cyclopropyl}-urea (isomer 1) and Example 170: 1-Spiro[3.3]hept-2-yl-3-{1-[3-(2,2,2-trifluoro-1-methyl-ethoxy)-phenyl]-cyclopropyl}-urea (isomer 2)

The racemate (±)-1-spiro[3.3]hept-2-yl-3-{1-[3-(2,2,2-trifluoro-1-methyl-ethoxy)-phenyl]-cyclopropyl}-urea was separated by the preparative chiral HPLC 1 method to give Example 169 (first eluting, t_{R}=9.5 min) and Example 170 (second eluting, t_{R}=11.3 min). Example 169 (LC-MS (1): t_{R}=1.24 min; [M+H]⁺: 383.2) and Example 170 (LC-MS (1): t_{R}=1.24 min; [M+H]⁺: 383.1).

### Example 171: 1-Spiro[3.3]hept-2-yl-3-[2-(2,2,2-trifluoro-1-methoxymethyl-ethoxy)-pyridin-4-ylmethyl]-urea (isomer 1) and Example 172: 1-Spiro[3.3]hept-2-yl-3-[2-(2,2,2-trifluoro-1-methoxymethyl-ethoxy)-pyridin-4-ylmethyl]-urea (isomer 2)

The racemate (±)-1-spiro[3.3]hept-2-yl-3-[2-(2,2,2-trifluoro-1-methoxymethyl-ethoxy)-pyridin-4-ylmethyl]-urea was separated by the preparative chiral SFC 3 method to give Example 171 (first eluting, t_{R}=5.7 min) and Example 172 (second eluting, t_{R}=7.5 min). Example 171 (LC-MS (1): t_{R}=1.14 min; [M+H]⁺: 388.2) and Example 172 (LC-MS (1): t_{R}=1.14 min; [M+H]⁺: 388.1).

### Example 173: 1-[(S)-1-(3-Difluoromethoxy-phenyl)-ethyl]-3-spiro[3.3]hept-2-yl-urea and

### Example 174: 1-[(R)-1-(3-Difluoromethoxy-phenyl)-ethyl]-3-spiro[3.3]hept-2-yl-urea

The racemate (±)-1-(1-(3-(difluoromethoxy)phenyl)ethyl)-3-(spiro[3.3]heptan-2-yl)urea (prepared as described for Example 12) was separated by the preparative chiral SFC 4 method to give Example 173 (first eluting, t_{R}=0.9 min) and Example 174 (second eluting, t_{R}=1.5 min). Example 173 (LC-MS (1): t_{R}=1.13 min; [M+H]⁺: 325.1) and Example 174 (LC-MS (1): t_{R}=1.13 min; [M+H]⁺: 325.1).

### Example 175: 1-Spiro[3.3]hept-2-yl-3-[(S)-1-(3-trifluoromethyl-phenyl)-ethyl]-urea

The racemate (±)-1-spiro[3.3]hept-2-yl-3-[1-(3-trifluoromethyl-phenyl)-ethyl]-urea was separated by the preparative chiral SFC 4 method to give Example 175 (first eluting, t_{R}=0.9 min) and 1-Spiro[3.3]hept-2-yl-3-[(R)-1-(3-trifluoromethyl-phenyl)-ethyl]-urea (second eluting, t_{R}=1.3 min). Example 175 (LC-MS (1): t_{R}=1.19 min; [M+H]⁺: 327.1).

### Example 176: 1-Spiro[3.3]hept-2-yl-3-{(S)-1-[3-(2,2,2-trifluoro-ethoxy)-phenyl]-ethyl}-urea

The racemate (±)-1-spiro[3.3]hept-2-yl-3-{1-[3-(2,2,2-trifluoro-ethoxy)-phenyl]-ethyl}-urea was separated by the preparative chiral SFC 1 method to give 1-spiro[3.3]hept-2-yl-3-{(R)-1-[3-(2,2,2-trifluoro-ethoxy)-phenyl]-ethyl}-urea (first eluting, t_{R}=4.3 min) and Example 176 (second eluting, t_{R}=9.9 min). Example 176 (LC-MS (1): t_{R}=1.18 min; [M+H]⁺: 357.1).

### Example 177: 1-{(S)-1-[4-Fluoro-3-(2,2,2-trifluoro-ethoxy)-phenyl]-ethyl}-3-spiro[3.3]hept-2-yl-urea

The racemate (±)-1-{1-[4-fluoro-3-(2,2,2-trifluoro-ethoxy)-phenyl]-ethyl}-3-spiro[3.3]hept-2-yl-urea was separated by the preparative chiral SFC 5 method to give 1-{(R)-1-[4-Fluoro-3-(2,2,2-trifluoro-ethoxy)-phenyl]-ethyl}-3-spiro[3.3]hept-2-yl-urea (first eluting, t_{R}=5.4 min) and Example 177 (second eluting, t_{R}=12.5 min). Example 177 (LC-MS (1): t_{R}=1.19 min; [M+H]⁺: 375.1).

### Example 178: 1-Spiro[3.3]hept-2-yl-3-[(R)-2,2,2-trifluoro-1-(3-trifluoromethyl-phenyl)-ethyl]-urea

The racemate (±)-1-spiro[3.3]hept-2-yl-3-[2,2,2-trifluoro-1-(3-trifluoromethyl-phenyl)-ethyl]-urea was separated by the preparative chiral SFC 6 method to give Example 178 (first eluting, t_{R}=1.2 min) and 1-spiro[3.3]hept-2-yl-3-[(S)-2,2,2-trifluoro-1-(3-trifluoromethyl-phenyl)-ethyl]-urea (second eluting, t_{R}=1.7 min). Example 178 (LC-MS (1): t_{R}=1.28 min; [M+H]⁺: 381.1).

### Example 179: 1-Spiro[3.3]hept-2-yl-3-[(R)-(1H-[1,2,4]triazol-3-yl)-(3-trifluoromethyl-phenyl)-methyl]-urea

The racemate (±)-1-spiro[3.3]hept-2-yl-3-[(1H-[1,2,4]triazol-3-yl)-(3-trifluoromethyl-phenyl)-methyl]-urea was separated by the preparative chiral SFC 7 method to give Example 179 (first eluting, t_{R}=1.3 min) and 1-spiro[3.3]hept-2-yl-3-[(S)-(1H-[1,2,4]triazol-3-yl)-(3-trifluoromethyl-phenyl)-methyl]-urea (second eluting, t_{R}=1.9 min). Example 179 (LC-MS (1): t_{R}=1.01 min; [M+H]⁺: 380.1).

### Example 180: 1-Spiro[3.3]hept-2-yl-3-[2-(-2,2,2-trifluoro-1-methyl-ethoxy)-pyrimidin-4-ylmethyl]-urea (isomer 1) and Example 181: 1-Spiro[3.3]hept-2-yl-3-[2-(2,2,2-trifluoro-1-methyl-ethoxy)-pyrimidin-4-ylmethyl]-urea (isomer 2)

The racemate (±)-1-spiro[3.3]hept-2-yl-3-[2-(2,2,2-trifluoro-1-methyl-ethoxy)-pyrimidin-4-ylmethyl]-urea was separated by the preparative chiral SFC 8 method to give Example 180 (first eluting, t_{R}=3.2 min) and Example 181 (second eluting, t_{R}=4.0 min). Example 180 (LC-MS (1): t_{R}=1.07 min; [M+H]⁺: 359.1) and Example 181 (LC-MS (1): t_{R}=1.07 min; [M+H]⁺: 359.1).

### Example 182: 1-Spiro[3.3]hept-2-yl-3-{(S)-1-[6-(2,2,2-trifluoro-ethoxy)-pyrimidin-4-yl]-ethyl}-urea and Example 183: 1-Spiro[3.3]hept-2-yl-3-{(R)-1-[6-(2,2,2-trifluoro-ethoxy)-pyrimidin-4-yl]-ethyl}-urea

The racemate (±)-1-spiro[3.3]hept-2-yl-3-{1-[6-(2,2,2-trifluoro-ethoxy)-pyrimidin-4-yl]-ethyl}-urea was separated by the preparative chiral SFC 9 method to give Example 182 (first eluting, t_{R}=1.1 min) and Example 183 (second eluting, t_{R}=1.5 min). Example 182 (LC-MS (1): t_{R}=1.08 min; [M+H]⁺: 359.1) and Example 183 (LC-MS (1): t_{R}=1.08 min; [M+H]⁺: 359.1).

### Example 184: 1-[(R)-2-Hydroxy-1-(3-trifluoromethyl-phenyl)-ethyl]-3-spiro[3.3]hept-2-yl-urea and Example 185: 1-[(S)-2-Hydroxy-1-(3-trifluoromethyl-phenyl)-ethyl]-3-spiro[3.3]hept-2-yl-urea

The racemate (±)-1-[2-hydroxy-1-(3-trifluoromethyl-phenyl)-ethyl]-3-spiro[3.3]hept-2-yl-urea was separated by the preparative chiral SFC 10 method to give Example 184 (first eluting, t_{R}=2.0 min) and Example 185 (second eluting, t_{R}=2.7 min). Example 184 (LC-MS (1): t_{R}=1.05 min; [M+H]⁺: 343.1) and Example 185 (LC-MS (1): t_{R}=1.05 min; [M+H]⁺: 343.1).

### Example 186: 1-[(R)-2-Hydroxy-1-(3-trifluoromethyl-phenyl)-ethyl]-3-spiro[2.3]hex-5-yl-urea

The racemate (±)-1-[2-Hydroxy-1-(3-trifluoromethyl-phenyl)-ethyl]-3-spiro[2.3]hex-5-yl-urea was separated by the preparative chiral SFC 11 method to give 1-[(S)-2-Hydroxy-1-(3-trifluoromethyl-phenyl)-ethyl]-3-spiro[2.3]hex-5-yl-urea (first eluting, t_{R}=1.0 min) and Example 186 (second eluting, t_{R}=1.5 min). Example 186 (LC-MS (1): t_{R}=0.97 min; [M+H]⁺: 329.1).

### Example 187: 1-[(R)-1-(2-Difluoromethoxy-pyridin-4-yl)-2-methoxy-ethyl]-3-spiro[3.3]hept-2-yl-urea

The racemate (±)-1-[1-(2-difluoromethoxy-pyridin-4-yl)-2-methoxy-ethyl]-3-spiro[3.3]hept-2-yl-urea was separated by the preparative chiral SFC 4 method to give Example 187 (first eluting, t_{R}=1.0 min) and 1-[(S)-1-(2-difluoromethoxy-pyridin-4-yl)-2-methoxy-ethyl]-3-spiro[3.3]hept-2-yl-urea (second eluting, t_{R}=1.5 min). Example 187 (LC-MS (1): t_{R}=1.07 min; [M+H]⁺: 356.1).

### Example 188: 1-{(R)-2-Methoxy-1-[2-(2,2,2-trifluoro-ethoxy)-pyridin-4-yl]-ethyl}-3-spiro[2.3]hex-5-yl-urea

The racemate (±)-1-{2-methoxy-1-[2-(2,2,2-trifluoro-ethoxy)-pyridin-4-yl]-ethyl}-3-spiro[2.3]hex-5-yl-urea was separated by the preparative chiral SFC 12 method to give Example 188 (first eluting, t_{R}=2.3 min) and 1-{(S)-2-methoxy-1-[2-(2,2,2-trifluoro-ethoxy)-pyridin-4-yl]-ethyl}-3-spiro[2.3]hex-5-yl-urea (second eluting, t_{R}=3.0 min). Example 188 (LC-MS (1): t_{R}=1.07 min; [M+H]⁺: 374.1).

### Example 189: 1-[(S)-1-(2-Difluoromethoxy-pyridin-4-yl)-ethyl]-3-spiro[2.3]hex-5-yl-urea

The racemate (±)-1-[1-(2-difluoromethoxy-pyridin-4-yl)-ethyl]-3-spiro[2.3]hex-5-yl-urea was separated by the preparative chiral SFC 13 method to give 1-[(R)-1-(2-difluoromethoxy-pyridin-4-yl)-ethyl]-3-spiro[2.3]hex-5-yl-urea (first eluting, t_{R}=1.5 min) and Example 189 (second eluting, t_{R}=2.1 min). Example 189 (LC-MS (1): t_{R}=0.99 min; [M+H]⁺: 312.1).

### Example 190: 1-[(S)-1-(2-Bromo-5-trifluoromethyl-phenyl)-ethyl]-3-spiro[3.3]hept-2-yl-urea

The racemate (±)-1-[1-(2-Bromo-5-trifluoromethyl-phenyl)-ethyl]-3-spiro[3.3]hept-2-yl-urea was separated by the preparative chiral SFC 14 method to give Example 190 (first eluting, t_{R}=1.1 min) and 1-[(R)-1-(2-Bromo-5-trifluoromethyl-phenyl)-ethyl]-3-spiro[3.3]hept-2-yl-urea (second eluting, t_{R}=1.6 min). Example 190 (LC-MS (1): t_{R}=1.28 min; [M+H]⁺: 405.0).

### Synthesis of 4-nitrophenyl (3-(trifluoromethyl)benzyl)carbamate

To an ice-cooled solution of 3-(trifluoromethyl)benzylamine (1.50 g, 8.4 mmol, 1 eq) and DIPEA (4.31 mL, 25.2 mmol, 3 eq) in THE (43 mL), 4-nitrophenyl chloroformate (1.74 g, 8.4 mmol, 1 eq) was added. The resulting mixture was stirred at 0° C. for 1 hour. The reaction mixture was diluted with water (25 mL) and EtOAc (25 mL). The layers were separated. The aq. phase was extracted with EtOAc (2×25 mL). The comb. org. phases were dried over MgSO₄ and concentrated in vacuo. The residue was purified by CombiFlash (column: 40 g, flow: 37 mL/min, Heptane 100% to Heptane+20% EtOAc) to afford a pale yellow solid which was further triturated in heptane/EtOAc 8:2 to yield a white solid. LC-MS (2): t_{R}=1.00 min; no ionization.

The following carbamates were synthesized using the appropriate amine or amine salt (HCl or TFA) derivatives and following the procedure described for 4-nitrophenyl (3-(trifluoromethyl)benzyl)carbamate. LC-MS data are listed in the table below. The LC-MS conditions used were LC-MS (2).

| Name | t_{R} | [M + H]⁺ |
|---|---|---|
| 4-nitrophenyl spiro[3.3]heptan-2-ylcarbamate | 1.00 | No ionization |
| 4-nitrophenyl ((2-(2,2,2-trifluoroethoxy)pyridin-4- | 0.97 | 372.15 |
| yl)methyl)carbamate | | |

### Synthesis of (±)-1-(2-bromo-5-(trifluoromethyl)phenyl)ethan-1-amine hydrochloride

### Step 1: (±,E)-N-(2-bromo-5-(trifluoromethyl)benzylidene)-2-methylpropane-2-sulfinamide

To a mixture of 2-bromo-5-(trifluoromethyl)benzaldehyde (1.27 g, 5 mmol, 1.0 eq) and (±)-2-methylpropane-2-sulfinamide (829 mg, 6.5 mmol, 1.3 eq) in THF (25 mL), titanium(IV) ethoxide (1.15 mL, 5.5 mmol, 1.1 eq) was added dropwise. The solution was stirred at rt for 20 hours. The yellow solution was diluted with water (50 mL) and DCM (100 mL). The resulting mixture was filtered. The layers were separated and the aq. phase was extracted with DCM (2×50 mL). The comb. org. phases were washed with H₂O (1×50 mL), sat. aq. NaCl soln. (1×50 mL), dried over MgSO₄, and concentrated in vacuo. The residue was purified by Combiflash (column: 40 g, flow: 40 mL/min, heptane to heptane/EtOAc 80:20) to yield a white-off solid. LC-MS (2): t_{R}=1.06 min; [M+H]⁺: 355.98.

### Step 2: (±)-N—((RS)-1-(2-bromo-5-(trifluoromethyl)phenyl)ethyl)-2-methylpropane-2-sulfinamide

To an ice-cooled solution of (±,E)-N-(2-bromo-5-(trifluoromethyl)benzylidene)-2-methylpropane-2-sulfinamide (1.04 g, 2.91 mmol, 1.0 eq) in THF (20 mL), 3M methylmagnesium bromide in diethyl ether (1.26 mL, 3.78 mmol, 1.3 eq) was added dropwise. The mixture was stirred at 0° C. for 4 hours. The reaction was carefully quenched with 1M aq. NH₄Cl (50 mL). The resulting suspension was diluted with water (50 mL) and EtOAc (50 mL). The layers were separated and the aq. phase was extracted with EtOAc (2×50 mL). The comb. org. phases were washed with sat. aq. NaCl soln. (50 mL), dried over MgSO₄, and concentrated in vacuo to give a pale yellow thick oil. The product was used without further purification. LC-MS (2): t_{R}=0.98 min; [M+H]⁺: 372.02.

### Step 3: (±)-1-(2-bromo-5-(trifluoromethyl)phenyl)ethan-1-amine hydrochloride

To an ice-cooled solution of (±)-N—((RS)-1-(2-bromo-5-(trifluoromethyl)phenyl)ethyl)-2-methylpropane-2-sulfinamide (1.08 g, 2.91 mmol, 1 eq) in MeOH (15 mL), 4M HCl in dioxane (2.9 mL) was added. The mixture was stirred at 0° C. for 3 hours. The mixture was concentrated in vacuo to give a yellow solid. The product was used without further purification. LC-MS (2): t_{R}=0.59 min; [M+H]⁺: 268.06.

The following amine salts were synthesized using the appropriate benzaldehyde derivative and following the procedure described for (±)-1-(2-bromo-5-(trifluoromethyl)phenyl)ethan-1-amine hydrochloride (Steps 1 to 3). LC-MS data are listed in the table below. The LC-MS conditions used were LC-MS (2).

| Name | t_{R} | [M + H]⁺ |
|---|---|---|
| (±)-1-(2-methoxy-5-(trifluoromethyl)phenyl)ethan-1-amine | 0.60 | 220.24 |
| hydrochloride | | |
| (±)-1-(2-methoxy-3-(trifluoromethyl)phenyl)ethan-1-amine | 0.58 | 220.24 |
| hydrochloride | | |
| (±)-1-(3-(trifluoromethyl)phenyl)ethan-1-amine hydrochloride | 0.55 | 190.31 |
| (±)-1-(4-methyl-3-(2,2,2-trifluoroethoxy)phenyl)ethan-1-amine | 0.67 | 234.17 |
| hydrochloride | | |
| (±)-1-(4-chloro-3-(2,2,2-trifluoroethoxy)phenyl)ethan-1-amine | 0.67 | 254.17 |
| hydrochloride | | |
| (±)-1-(4-methoxy-3-(2,2,2-trifluoroethoxy)phenyl)ethan-1-amine | 0.58 | 250.21 |
| hydrochloride | | |
| (±)-1-(2-methyl-3-(2,2,2-trifluoroethoxy)phenyl)ethan-1-amine | 0.63 | 234.20 |
| hydrochloride | | |
| (±)-1-(2-chloro-3-(2,2,2-trifluoroethoxy)phenyl)ethan-1-amine | 0.62 | 254.17 |
| hydrochloride | | |
| (±)-1-(2-methoxy-3-(2,2,2-trifluoroethoxy)phenyl)ethan-1-amine | 0.61 | 250.20 |
| hydrochloride | | |
| (±)-1-(3-methyl-5-(2,2,2-trifluoroethoxy)phenyl)ethan-1-amine | 0.65 | 234.20 |
| hydrochloride | | |
| (±)-1-(3-chloro-5-(2,2,2-trifluoroethoxy)phenyl)ethan-1-amine | 0.66 | 254.15 |
| hydrochloride | | |
| (±)-1-(3-(pentafluoro-λ⁶-sulfaneyl)phenyl)ethan-1-amine hydrochloride | 0.60 | 248.16 |
| (±)-1-(4-fluoro-3-(2,2,2-trifluoroethoxy)phenyl)ethan-1-amine | 0.61 | 238.16 |
| hydrochloride | | |
| (±)-1-(3-fluoro-5-(2,2,2-trifluoroethoxy)phenyl)ethan-1-amine | 0.61 | 238.17 |
| hydrochloride | | |
| (±)-1-(2-(trifluoromethoxy)pyridin-4-yl)ethan-1-amine hydrochloride | 0.47 | 207.26 |
| (±)-1-(2-fluoro-5-(2,2,2-trifluoroethoxy)phenyl)ethan-1-amine | 0.59 | 238.16 |
| hydrochloride | | |
| (±)-1-(2-fluoro-3-(2,2,2-trifluoroethoxy)phenyl)ethan-1-amine | 0.58 | 238.17 |
| hydrochloride | | |
| (±)-1-(2-methoxy-5-(2,2,2-trifluoroethoxy)phenyl)ethan-1-amine | 0.63 | 250.22 |
| hydrochloride | | |
| (±)-1-(2-chloro-5-(2,2,2-trifluoroethoxy)phenyl)ethan-1-amine | 0.64 | 254.16 |
| hydrochloride | | |
| (±)-1-(2,2-difluorobenzo[d][1,3]dioxol-4-yl)ethan-1-amine hydrochloride | 0.54 | 202.26 |
| (±)-1-(3-(difluoromethoxy)phenyl)ethan-1-amine hydrochloride | 0.51 | 188.32 |

### Synthesis of (±)-cyclopropyl(3-(trifluoromethyl)phenyl)methanamine hydrochloride

### Step 1: (±,E)-2-methyl-N-(3-(trifluoromethyl)benzylidene)propane-2-sulfinamide

To a mixture of 3-(trifluoromethyl)benzaldehyde (1.80 g, 10 mmol, 1.0 eq) and (±)-2-methyl-2-propanesulfinamide (1.62 g, 13 mmol, 1.3 eq) in THF (50 mL), titanium(IV) ethoxide (2.31 mL, 11 mmol, 1.1 eq) was added dropwise. The solution was stirred at rt for 20 hours. The yellow solution was diluted with water (100 mL) and DCM (150 mL). The resulting mixture was filtered. The layers were separated and the aq. phase was extracted with DCM (2×50 mL). The comb. org. phases were washed with H₂O (1×100 mL), sat. aq. NaCl soln. (1×100 mL), dried over MgSO₄, and concentrated in vacuo. The residue was purified by Combiflash (column: 80 g, flow: 60 mL/min, heptane to heptane/EtOAc 100:25) to give a white solid. LC-MS (2): t_{R}=1.00 min; [M+H]⁺: 278.23.

### Step 2: (±)-N—((RS)-cyclopropyl(3-(trifluoromethyl)phenyl)methyl)-2-methylpropane-2-sulfinamide

To an ice-cooled solution of (±,E)-2-methyl-N-(3-(trifluoromethyl)benzylidene)propane-2-sulfinamide (832 mg, 3.0 mmol, 1.0 eq) in THF (15 mL), 1M cyclopropylmagnesium bromide in 2-methyltetrahydrofuran (3.9 mL, 3.9 mmol, 1.3 eq) was added dropwise. The mixture was stirred at 0° C. for 6 hours. The reaction was carefully quenched with 1M aq. NH₄Cl soln. (30 mL). The resulting suspension was diluted with water (30 mL) and EtOAc (60 mL). The layers were separated and the aq. phase was extracted with EtOAc (2×60 mL). The comb. org. phases were washed with sat. aq. NaCl soln. (60 mL), dried over MgSO₄, and concentrated in vacuo to give a yellow oil. The product was used without further purification. LC-MS (2): t_{R}=0.96 min; [M+H]⁺: 320.27.

### Step 3: (±)-cyclopropyl(3-(trifluoromethyl)phenyl)methanamine hydrochloride

To an ice-cooled solution of (±)-N—((RS)-cyclopropyl(3-(trifluoromethyl)phenyl)methyl)-2-methylpropane-2-sulfinamide (128 mg, 0.4 mmol, 1 eq) in MeOH (4 mL), 4M HCl in dioxane (0.2 mL) was added. The mixture was stirred at rt for 18 hours. The mixture was concentrated in vacuo to give a pale yellow solid. The product was used without further purification. LC-MS (2): t_{R}=0.62 min; [M+H]⁺: 216.26.

The following amine salts were synthesized using the appropriate benzaldehyde derivative in step 1 and the appropriate Grignard reagent in step 2 and following the procedure described for (±)-1-(2-bromo-5-(trifluoromethyl)phenyl)ethan-1-amine hydrochloride (Steps 1 to 3). LC-MS data are listed in the table below.

| | | | LC-MS |
|---|---|---|---|
| Name | t_{R} | [M + H]⁺ | conditions |
| (±)-cyclopropyl(3-(difluoromethoxy)phenyl)methanamine | 0.58 | 216.26 | 2 |
| hydrochloride | | | |
| 1-(3-(trifluoromethyl)phenyl)prop-2-yn-1-amine | 0.84 | 200.28 | 3 |
| hydrochloride | | | |

### Synthesis of (2-methoxy-3-(trifluoromethyl)phenyl)methanamine hydrochloride

### Step 1: (±, E)-N-(2-methoxy-3-(trifluoromethyl)benzylidene)-2-methylpropane-2-sulfinamide

To a mixture of 2-methoxy-3-(trifluoromethyl)benzaldehyde (1.07 g, 5 mmol, 1 eq) and (±)-2-methylpropane-2-sulfinamide (829 mg, 6.5 mmol, 1.3 eq) in THE (25 mL), titanium(IV) ethoxide (1.15 mL, 5.5 mmol, 1.1 eq) was added dropwise. The solution was stirred at rt for 22 hours. The yellow solution was diluted with water (50 mL) and DCM (100 mL). The resulting mixture was filtered. The layers were separated and the aq. phase was extracted with DCM (2×50 mL). The comb. org. phases were washed with H₂O (1×50 mL), sat. aq. NaCl soln. (1×50 mL), dried over MgSO₄, and concentrated in vacuo. The residue was purified by Combiflash (column: 40 g, flow: 40 mL/min, heptane to heptane/EtOAc 60:40) to give a yellow oil that solidified upon standing. LC-MS (2): t_{R}=1.02 min; [M+H]⁺: 308.20.

### Step 2: (±)-N-(2-methoxy-3-(trifluoromethyl)benzyl)-2-methylpropane-2-sulfinamide

To an ice-cooled solution of (±,E)-N-(2-methoxy-3-(trifluoromethyl)benzylidene)-2-methylpropane-2-sulfinamide (760 mg, 2.47 mmol, 1.0 eq) in MeOH (20 mL), NaBH₄ (140 mg, 3.71 mmol, 1.5 eq) was added. The mixture was stirred at 0° C. for 3 hours. The reaction mixture was concentrated in vacuo. The residue was partitioned between water (50 mL) and DCM (50 mL). The layers were separated. The aq. phase was extracted with DCM (2×50 mL). The comb. org. phases were dried over MgSO₄ and concentrated in vacuo to give a white solid. The product was used without further purification. LC-MS (2): t_{R}=0.90 min; [M+H]⁺: 310.22.

### Step 3: (2-methoxy-3-(trifluoromethyl)phenyl)methanamine hydrochloride

To an ice-cooled solution of (±)-N-(2-methoxy-3-(trifluoromethyl)benzyl)-2-methylpropane-2-sulfinamide (745 mg, 2.41 mmol, 1 eq) in MeOH (15 mL), 4M HCl in dioxane (2.4 mL) was added. The mixture was stirred at 0° C. for 3 hours. The mixture was concentrated in vacuo to give a pale yellow solid. The product was used without further purification. LC-MS (2): t_{R}=0.54 min; [M+H]⁺: 206.28.

The following amine salts were synthesized using the appropriate benzaldehyde derivative and following the procedure described for (2-methoxy-3-(trifluoromethyl)phenyl)methanamine hydrochloride (Steps 1 to 3). LC-MS data are listed in the table below. The LC-MS conditions used were LC-MS (2).

| Name | t_{R} | [M + H]⁺ |
|---|---|---|
| (3-(pentafluoro-λ⁶-sulfaneyl)phenyl)methanamine hydrochloride | 0.57 | 234.13 |
| (4-fluoro-3-(2,2,2-trifluoroethoxy)phenyl)methanamine hydrochloride | 0.57 | 224.21 |
| (2-(trifluoromethoxy)pyridin-4-yl)methanamine hydrochloride | 0.40 | 193.28 |
| (2,2-difluorobenzo[d][1,3]dioxol-4-yl)methanamine hydrochloride | 0.48 | 188.30 |
| (2-methoxy-5-(trifluoromethyl)phenyl)methanamine hydrochloride | 0.56 | 206.28 |

### Synthesis of (4-methyl-3-(2,2,2-trifluoroethoxy)phenyl)methanamine hydrochloride

### Step 1: (±, E)-N-(4-bromo-3-(2,2,2-trifluoroethoxy)benzylidene)-2-methylpropane-2-sulfinamide

To a mixture of 4-bromo-3-(2,2,2-trifluoroethoxy)benzaldehyde (1.35 g, 4.77 mmol, 1.00 eq) and (±)-2-methyl-2-propanesulfinamide (636 mg, 5.25 mmol, 1.10 eq) in THE (50 mL), titanium (IV) ethoxide (5.73 mL, 5.47 mmol, 1.15 eq) was added dropwise. The reaction mixture was stirred at rt overnight. The reaction mixture was diluted with water (100 mL) and DCM (100 mL). The reaction mixture was filtered. The layers were separated. The aq. phase was extracted with DCM (2×50 mL). The comb. org. phases were washed with water (1×25 mL), sat. aq. NaCl soln. (1×25 mL), dried over MgSO₄, and concentrated in vacuo. The residue was used without further purification. LC-MS (2): t_{R}=1.08 min; [M+H]⁺: 385.87.

### Step 2: (±)-N-(4-bromo-3-(2,2,2-trifluoroethoxy)benzyl)-2-methylpropane-2-sulfinamide

To an ice-cooled solution of (±,E)-N-(4-bromo-3-(2,2,2-trifluoroethoxy)benzylidene)-2-methylpropane-2-sulfinamide (1.8 g, 4.66 mmol, 1.0 eq) in MeOH (20 mL), sodium borohydride (264 mg, 6.99 mmol, 1.5 eq) was added. The mixture was stirred at 0° C. for 2 hours. The reaction mixture was concentrated in vacuo. The residue was partitioned between water (25 mL) and DCM (25 mL). The layers were separated. The aq. phase was extracted with DCM (2×25 mL). The comb. org. phases were dried over MgSO₄ and concentrated in vacuo. The residue was purified by CombiFlash (column: 40 g, flow: 40 mL/min, Heptane+15% EtOAc to EtOAc) to yield a colorless oil. LC-MS (2): t_{R}=0.96 min; [M+H]⁺: 388.01.

### Step 3: (±)-2-methyl-N-(4-methyl-3-(2,2,2-trifluoroethoxy)benzyl)propane-2-sulfinamide

To a solution under N₂ of (±)-N-(4-bromo-3-(2,2,2-trifluoroethoxy)benzyl)-2-methylpropane-2-sulfinamide (300 mg, 0.77 mmol, 1.00 eq) in THF (4 mL). tetrakis(triphenylphosphine)palladium(0) (45 mg, 0.04 mmol, 0.05 eq) and 1M dimethylzinc solution in heptane (3 mL, 3.09 mmol, 4.00 eq) were added in sequence. The mixture was stirred at 50° C. overnight. The mixture was allowed to cool to rt and concentrated in vacuo. The residue was diluted with DCM (25 mL) and washed with water (2×15 mL). The org. layer was dried over MgSO₄ and concentrated in vacuo. The residue was purified by CombiFlash (column: 24 g, flow: 35 mL/min, Heptane+30% EtOAc to EtOAc) to yield a brown oil. LC-MS (2): t_{R}=0.96 min; [M+H]⁺: 324.81.

### Step 4: (4-methyl-3-(2,2,2-trifluoroethoxy)phenyl)methanamine hydrochloride

To an ice-cooled solution of (±)-2-methyl-N-(4-methyl-3-(2,2,2-trifluoroethoxy)benzyl)propane-2-sulfinamide (215 mg, 0.67 mmol, 1 eq.) in DCM (4 mL), 4M HCl in dioxane (0.83 mL, 3.32 mmol, 5 eq) was added. The resulting solution was stirred at rt overnight. The reaction mixture was concentrated in vacuo. The residue was used without further purification. LC-MS (2): t_{R}=0.64 min; [M+H]⁺: 220.22.

### Synthesis of (5-methyl-6-(2,2,2-trifluoroethoxy)pyridin-2-yl)methanamine hydrochloride

### Step 1: (±, E)-N-((5-bromo-6-fluoropyridin-2-yl)methylene)-2-methylpropane-2-sulfinamide

To a mixture of 5-bromo-6-fluoropyridine-2-carbaldehyde (500 mg, 2.33 mmol, 1.0 eq) and (±)-2-methyl-2-propanesulfinamide (310 mg, 2.56 mmol, 1.1 eq) in THF (50 mL), titanium (IV) ethoxide (2.8 mL, 2.67 mmol, 1.15 eq) was added dropwise. The reaction mixture was stirred at rt overnight. The reaction mixture was diluted with water (100 mL) and DCM (100 mL). The reaction mixture was filtered. The layers were separated. The aq. phase was extracted with DCM (2×50 mL). The comb. org. phases were washed with water (1×25 mL), sat. aq. NaCl soln. (1×25 mL), dried over MgSO₄, and concentrated in vacuo. The residue was used without further purification. LC-MS (2): t_{R}=0.92 min; [M+H]⁺: 307.06.

### Step 2: (±, E)-N-((5-bromo-6-(2,2,2-trifluoroethoxy)pyridin-2-yl)methylene)-2-methylpropane-2-sulfinamide

To an ice-cooled solution of 2,2,2-trifluoroethanol (0.13 mL, 1.76 mmol, 1.2 eq) in THF (10 mL), sodium hydride 60% dispersion in mineral oil (76.3 mg, 1.91 mmol, 1.3 eq) was added. The mixture was stirred at 0° C. for 30 min. Then (±,E)-N-((5-bromo-6-fluoropyridin-2-yl)methylene)-2-methylpropane-2-sulfinamide (451 mg, 1.47 mmol, 1 eq) was added. Upon addition completion, the cooling bath was removed and the mixture was heated at 80° C. overnight. The reaction mixture was allowed to cool to rt and was partitioned between water and EtOAc (25 mL). The layers were separated. The org. phase was dried over MgSO₄ and concentrated in vacuo. The residue was purified by CombiFlash (column: 24 g, flow: 35 mL/min, Heptane to Heptane+28% EtOAc) to yield a brown oil. LC-MS (2): t_{R}=1.07 min; [M+H]⁺: 386.96.

### Step 3: (±)-N-((5-bromo-6-(2,2,2-trifluoroethoxy)pyridin-2-yl)methyl)-2-methylpropane-2-sulfinamide

To an ice-cooled solution of (±,E)-N-((5-bromo-6-(2,2,2-trifluoroethoxy)pyridin-2-yl)methylene)-2-methylpropane-2-sulfinamide (254 mg, 0.66 mmol, 1.0 eq) in MeOH (10 mL), NaBH₄ (37 mg, 0.98 mmol, 1.5 eq) was added. The mixture was stirred at 0° C. for 2 hours. The reaction mixture was concentrated in vacuo. The residue was partitioned between water (25 mL) and DCM (25 mL). The layers were separated. The aq. phase was extracted with DCM (2×25 mL). The comb. org. phases were dried over MgSO₄ and concentrated in vacuo. The product was used without further purification. LC-MS (2): t_{R}=0.96 min; [M+H]⁺: 389.00.

### Step 4: (±)-2-methyl-N-((5-methyl-6-(2,2,2-trifluoroethoxy)pyridin-2-yl)methyl)propane-2-sulfinamide

To a solution under N₂ of (±)-N-((5-bromo-6-(2,2,2-trifluoroethoxy)pyridin-2-yl)methyl)-2-methylpropane-2-sulfinamide (250 mg, 0.64 mmol, 1.00 eq) in THF (10 mL), tetrakis(triphenylphosphine)palladium(0) (37 mg, 0.03 mmol, 0.05 eq) and 1M dimethylzinc solution in heptane (2.57 mL, 2.57 mmol, 4.00 eq) were added in sequence. The mixture was stirred at 50° C. overnight. The mixture was allowed to cool to rt and concentrated in vacuo. The residue was diluted with DCM (25 mL) and washed with water (2×15 mL). The org. layer was dried over MgSO₄ and concentrated in vacuo. The residue was purified by CombiFlash (column: 24 g, flow: 35 mL/min, Heptane to Heptane+90% EtOAc) to yield an yellow oil. LC-MS (2): t_{R}=0.96 min; [M+H]⁺: 325.22.

### Step 5: (5-methyl-6-(2,2,2-trifluoroethoxy)pyridin-2-yl)methanamine hydrochloride

To an ice-cooled solution of (±)-2-methyl-N-((5-methyl-6-(2,2,2-trifluoroethoxy)pyridin-2-yl)methyl)propane-2-sulfinamide (113 mg, 0.35 mmol, 1 eq) in DCM (4 mL), 4M HCl in dioxane (0.44 mL, 1.74 mmol, 5 eq) was added. The resulting solution was stirred at rt overnight. The reaction mixture was concentrated in vacuo. The residue was used without further purification. LC-MS (2): t_{R}=0.62 min; [M+H]⁺: 221.24.

### Synthesis of 4-methyl-3-(2,2,2-trifluoroethoxy)benzaldehyde

To an ice-cooled suspension of 3-hydroxy-4-methylbenzaldehyde (485 mg, 3.49 mmol, 1.0 eq) and cesium carbonate (2.27 g, 6.98 mmol, 2 eq) in DMF (11 mL), 2,2,2-trifluoroethyl trifluoromethanesulfonate (0.77 mL, 5.23 mmol, 1.5 eq) was added. The resulting mixture was stirred at 0° C. for 30 min and further at rt for 1 hour. The reaction mixture was quenched with water and extracted with EtOAc (3×). The comb. org. phases were washed with sat. aq. NaCl soln., dried over MgSO₄ and evaporated. The residue was purified by Combiflash (column: 12 g, flow: 25 mL/min, heptane/EtOAc 8:2) to afford a slightly yellow solid. LC-MS (2): t_{R}=0.94 min; no ionization.

The following aldehydes were synthesized using the appropriate phenol derivative and following the procedure described for 4-methyl-3-(2,2,2-trifluoroethoxy)benzaldehyde. LC-MS data are listed in the table below. The LC-MS conditions used were LC-MS (2).

| Name | t_{R} | [M + H]⁺ |
|---|---|---|
| 4-chloro-3-(2,2,2-trifluoroethoxy)benzaldehyde | 0.94 | No ionization |
| 4-methoxy-3-(2,2,2-trifluoroethoxy)benzaldehyde | 0.86 | No ionization |
| 2-methyl-3-(2,2,2-trifluoroethoxy)benzaldehyde | 0.93 | No ionization |
| 2-chloro-3-(2,2,2-trifluoroethoxy)benzaldehyde | 0.93 | No ionization |
| 2-methoxy-3-(2,2,2-trifluoroethoxy)benzaldehyde | 0.90 | No ionization |
| 3-methyl-5-(2,2,2-trifluoroethoxy)benzaldehyde | 0.93 | No ionization |
| 3-chloro-5-(2,2,2-trifluoroethoxy)benzaldehyde | 0.95 | No ionization |
| 4-fluoro-3-(2,2,2-trifluoroethoxy)benzaldehyde | 0.90 | No ionization |
| 3-fluoro-5-(2,2,2-trifluoroethoxy)benzaldehyde | 0.92 | No ionization |
| 2-fluoro-5-(2,2,2-trifluoroethoxy)benzaldehyde | 0.91 | No ionization |
| 2-fluoro-3-(2,2,2-trifluoroethoxy)benzaldehyde | 0.89 | No ionization |
| 4-bromo-3-(2,2,2-trifluoroethoxy)benzaldehyde | 0.95 | No ionization |
| 2-methoxy-5-(2,2,2-trifluoroethoxy)benzaldehyde | 0.91 | No ionization |
| 2-chloro-5-(2,2,2-trifluoroethoxy)benzaldehyde | 0.95 | No ionization |

### Synthesis of (±)-1-(3-(methylsulfonyl)phenyl)ethan-1-amine trifluoroacetate

### Step 1: tert-butyl (±)-(1-(3-(methylsulfonyl)phenyl)ethyl)carbamate

To a solution of 3-bromophenylmethylsulfone (353 mg, 1.50 mmol, 1.00 eq) in anh. DMSO (20 mL), Boc-DL-alanine (430 mg, 2.25 mmol, 1.50 eq), K₃PO₄ (975 mg, 4.50 mmol, 3.00 eq), 4,4′-di-tert-butyl-2,2′-dipyridyl (41 mg, 0.15 mmol, 0.10 eq), NiCl₂.glyme (34 mg, 0.15 mmol, 0.10 eq) and [Ir{dF(CF₃)ppy}₂(dtbpy)]PF₆ (34 mg, 0.03 mmol, 0.02 eq) were added in sequence. The resulting mixture was degassed with N2 while stirring for 15 minutes. Then the resulting mixture was stirred at rt overnight under blue LED irradiation. Water was added and the mixture was extracted with EtOAc (3×). The comb. org. layers were dried over MgSO₄ and concentrated in vacuo. The residue was purified by prep. HPLC (column: Waters XBridge, 50×150 mm, 10 um, UV/MS, basic conditions) to give a white solid. LC-MS (2): t_{R}=0.81 min; no ionization.

### Step 2: (±)-1-(3-(methylsulfonyl)phenyl)ethan-1-amine trifluoroacetate

To an ice-cooled solution of tert-butyl (±)-(1-(3-(methylsulfonyl)phenyl)ethyl)carbamate (45 mg, 0.15 mmol, 1 eq) in DCM (5 mL), trifluoroacetic acid (0.12 mL, 1.50 mmol, 10 eq) was added. The resulting solution was stirred at rt overnight. The reaction mixture was concentrated in vacuo to give a white solid. The residue was used without further purification. LC-MS (2): t_{R}=0.37 min; [M+H]⁺: 200.27.

The following amine salts were synthesized using the appropriate bromide and Boc-protected amino acid derivatives and following the procedure described for (±)-1-(3-(methylsulfonyl)phenyl)ethan-1-amine trifluoroacetate (Steps 1 and 2). LC-MS data are listed in the table below. The LC-MS conditions used were LC-MS (2).

| Name | t_{R} | [M + H]⁺ |
|---|---|---|
| (±)-1-(3-(2,2,2-trifluoroethoxy)phenyl)ethan-1- | 0.59 | 220.22 |
| amine trifluoroacetate | | |
| (±)-2-methoxy-1-(3-(2,2,2- | 0.60 | 250.17 |
| trifluoroethoxy)phenyl)ethan-1-amine trifluoroacetate | | |
| (±)-2-methoxy-1-(2-(2,2,2-trifluoroethoxy)pyridin-4- | 0.56 | 251.18 |
| yl)ethan-1-amine trifluoroacetate | | |
| (±)-1-(2-(2,2,2-trifluoroethoxy)pyridin-4-yl)ethan-1- | 0.54 | 221.21 |
| amine trifluoroacetate | | |
| (±)-N¹,N¹-dimethyl-2-(2-(2,2,2- | 0.35 | 264.24 |
| trifluoroethoxy)pyridin-4-yl)ethane-1,2-diamine | | |
| trifluoroacetate | | |
| (±)-N¹,N¹-dimethyl-2-(6-(2,2,2- | 0.35 | 264.26 |
| trifluoroethoxy)pyridin-2-yl)ethane-1,2-diamine | | |
| trifluoroacetate | | |
| (±)-1-(2-(difluoromethoxy)pyridin-4-yl)-2- | 0.47 | 219.23 |
| methoxyethan-1-amine trifluoroacetate | | |
| 1-(6-(2,2,2-trifluoroethoxy)pyrimidin-4-yl)ethan-1- | 0.48 | 222.25 |
| amine trifluoroacetate | | |

### Synthesis of (±)-2-isopropoxy-1-(3-(trifluoromethyl)phenyl)ethan-1-amine hydrochloride

### Step 1: tert-butyl (±)-(2-isopropoxy-1-(3-(trifluoromethyl)phenyl)ethyl)carbamate

To a solution of 3-bromobenzotrifluoride (0.22 mL, 1.5 mmol, 1.00 eq) in anh. DMSO (20 mL), 2-{[(tert-butoxy)carbonyl]amino-3-(propan-2-yloxy)propanoic acid (586 mg, 2.25 mmol, 1.50 eq), K₃PO₄ (975 mg, 4.5 mmol, 3.00 eq), 4,4′-di-tert-butyl-2,2′-dipyridyl (41 mg, 0.15 mmol, 0.10 eq), NiCl₂.glyme (34 mg, 0.15 mmol, 0.10 eq) and [Ir{dF(CF₃)ppy₂(dtbpy)]PF₆ (34 mg, 0.03 mmol, 0.02 eq) were added in sequence. The resulting mixture was degassed with N₂ while stirring for 15 minutes. The resulting mixture was stirred at rt overnight under blue LED irradiation. Water was added and the mixture was extracted with EtOAc (3×). The comb. org. layers were dried over MgSO₄, filtered and concentrated in vacuo. The residue was purified by prep. HPLC (column: Waters X-Bridge, 50×150 mm, 10 um, UV/MS, basic conditions) to give a white solid. LC-MS (2): t_{R}=1.08 min; [M+H]⁺: 348.25.

### Step 2: (±)-2-isopropoxy-1-(3-(trifluoromethyl)phenyl)ethan-1-amine hydrochloride

To an ice-cooled solution of tert-butyl (±)-(2-isopropoxy-1-(3-(trifluoromethyl)phenyl)ethyl)carbamate (52 mg, 0.15 mmol, 1 eq) in DCM (5 mL), 4M HCl in dioxane (57 μL) was added. The resulting solution was stirred at rt overnight. The reaction mixture was concentrated in vacuo. The residue was used without further purification. LC-MS (2): t_{R}=0.68 min; [M+H]⁺: 248.23.

The following amine salts were synthesized using the appropriate bromide and Boc-protected amino acid derivatives and following the procedure described for (±)-2-isopropoxy-1-(3-(trifluoromethyl)phenyl)ethan-1-amine hydrochloride (Steps 1 and 2). LC-MS data are listed in the table below. The LC-MS conditions used were LC-MS (2).

| Name | t_{R} | [M + H]⁺ |
|---|---|---|
| (±)-3-amino-3-(3-(trifluoromethyl)phenyl)propan-1-ol | 0.50 | 220.23 |
| hydrochloride | | |
| (±)-3-ethoxy-1-(3-(trifluoromethyl)phenyl)propan-1- | 0.65 | 248.23 |
| amine hydrochloride | | |
| (±)-3-(methylthio)-1-(3- | 0.65 | 250.18 |
| (trifluoromethyl)phenyl)propan-1-amine hydrochloride | | |
| (±)-2-amino-2-(3-(trifluoromethyl)phenyl)ethan-1-ol | 0.48 | 206.28 |
| hydrochloride | | |
| (±)-3-ethoxy-1-(2-(2,2,2-trifluoroethoxy)pyridin-4- | 0.63 | 279.23 |
| yl)propan-1-amine hydrochloride | | |
| (±)-2-methoxy-1-(6-(2,2,2-trifluoroethoxy)pyrimidin- | 0.51 | 252.19 |
| 4-yl)ethan-1-amine hydrochloride | | |
| (±)-4-amino-4-(3-(trifluoromethyl)phenyl)butanamide | 0.47 | 247.21 |
| hydrochloride | | |
| (±)-1-(3-(trifluoromethyl)phenyl)but-3-en-1-amine | 0.63 | 216.25 |
| hydrochloride | | |
| (±)-3-(methylsulfonyl)-1-(3- | 0.52 | 282.22 |
| (trifluoromethyl)phenyl)propan-1-amine hydrochloride | | |
| (±)-2-amino-2-(2-methyl-6-(2,2,2- | 0.49 | 252.19 |
| trifluoroethoxy)pyrimidin-4-yl)ethan-1-ol | | |
| hydrochloride | | |
| (±)-2-amino-2-(6-(2,2,2-trifluoroethoxy)pyrimidin- | 0.40 | 238.18 |
| 4-yl)ethan-1-ol hydrochloride | | |
| (±)-3,3-difluoro-1-(3- | 0.60 | 240.17 |
| (trifluoromethyl)phenyl)propan-1-amine hydrochloride | | |
| (±)-3-amino-3-(6-(2,2,2-trifluoroethoxy)pyrimidin- | 0.45 | 252.22 |
| 4-yl)propan-1-ol hydrochloride | | |
| (±)-1-(3-(methylsulfonyl)phenyl)ethan-1-amine | 0.37 | 200.27 |
| hydrochloride | | |
| (±)-2-methoxy-1-(3-(methylsulfonyl)phenyl)ethan- | 0.40 | 230.17 |
| 1-amine hydrochloride | | |
| (±)-2-amino-2-(3-(2,2,2-trifluoroethoxy)phenyl)ethan- | 0.53 | 235.63 |
| 1-ol hydrochloride | | |
| (±)-3-amino-3-(3-(2,2,2- | 0.55 | 250.23 |
| trifluoroethoxy)phenyl)propan-1-ol hydrochloride | | |

### Synthesis of 4-bromo-6-(2,2,2-trifluoroethoxy)pyrimidine

To a suspension of 4-bromo-6-hydroxypyrimidine (2.0 g, 11.1 mmol, 1.00 eq) in DMF (10 mL), Cs₂CO₃ (7.2 g, 22.2 mmol, 2.00 eq) and 2,2,2-trifluoroethyl trifluoromethanesulfonate (1.7 mL, 11.6 mmol, 1.05 eq) were added in sequence. The resulting mixture was stirred at rt overnight. The mixture was concentrated in vacuo. The residue was diluted with water (100 mL) and EtOAC (50 mL). The layers were separated. The aq. phase was extracted with EtOAc (2×50 mL). The comb. org. phases were washed with sat. aq. NaCl soln. (1×50 mL), dried over MgSO₄, and concentrated in vacuo. The residue was purified by Combiflash (column: 40 g, flow: 40 mL/min, Heptane to Heptane+12% EtOAc) to yield a colorless oil. LC-MS (2): t_{R}=0.81 min; no ionization.

The following bromide was synthesized using the appropriate hydroxy derivatives and following the procedure described for 4-bromo-6-(2,2,2-trifluoroethoxy)pyrimidine. LC-MS data are listed in the table below. The LC-MS conditions used were LC-MS (2).

| Name | t_{R} | [M + H]⁺ |
|---|---|---|
| 4-bromo-2-methyl-6-(2,2,2-trifluoroethoxy)pyrimidine | 0.92 | 271.08 |

### Synthesis of 4-(2-(2,2,2-trifluoroethoxy)pyridin-4-yl)tetrahydro-2H-pyran-4-amine trifluoroacetate

### Step 1: ten-butyl (4-(2-(2,2,2-trifluoroethoxy)pyridin-4-yl)tetrahydro-2H-pyran-4-yl)carbamate

To a vial of 2-(2,2,2-trifluoroethoxy)isonicotinonitrile (64 mg, 0.3 mmol, 1 eq) in DMA (15 mL), 4-(boc-amino)tetrahydropyran-4-carboxylic acid (228 mg, 0.9 mmol, 3 eq), cesium fluoride (140 mg, 0.9 mmol, 3 eq) and Ir(p-F(t-Bu)-ppy)₃ (5.3 mg, 0.006 mmol, 0.02 eq) were added in sequence. The resulting mixture was degassed with N2 while stirring for 15 minutes. Then, the resulting mixture was stirred at rt overnight under blue LED irradiation. Water was added and the mixture was extracted with EtOAc (3×). The comb. org. layers were dried over MgSO₄, filtered and concentrated in vacuo. The residue was purified by prep. HPLC (column: Waters XBridge, 30×75 mm, 10 um, UV/MS, basic conditions) to give an yellow oil. LC-MS (2): t_{R}=0.96 min; [M+H]⁺: 377.21.

### Step 2: 4-(2-(2,2,2-trifluoroethoxy)pyridin-4-yl)tetrahydro-2H-pyran-4-amine trifluoroacetate

To an ice-cooled solution of tert-butyl (4-(2-(2,2,2-trifluoroethoxy)pyridin-4-yl)tetrahydro-2H-pyran-4-yl)carbamate (40 mg, 0.1 mmol, 1 eq) in DCM (5 mL), trifluoroacetic acid (81 μL, 1 mmol, 10 eq) was added. The resulting solution was stirred at rt overnight. The reaction mixture was concentrated in vacuo. The residue was used without further purification. LC-MS (2): t_{R}=0.56 min; [M+H]⁺: 277.25.

### Synthesis of 2-(2-(difluoromethoxy)pyridin-4-yl)propan-2-amine

### Step 1: tert-butyl (2-(2-(difluoromethoxy)pyridin-4-yl)propan-2-yl)carbamate

To a vial of 2-(difluoromethoxy)pyridine-4-carbonitrile (111 mg, 0.62 mmol, 1 eq) in DMSO (15 mL), boc-2-aminoisobutyric acid (378 mg, 1.86 mmol, 3 eq), cesium fluoride (283 mg, 1.86 mmol, 3 eq) and Ir(p-F(t-Bu)-ppy)₃) (11 mg, 0.012 mmol, 0.02 eq) were added in sequence. The resulting mixture was degassed with N2 while stirring for 15 minutes. Then, the resulting mixture was stirred at rt overnight under blue LED irradiation. Water was added and the mixture was extracted with EtOAc (3×). The comb. org. layers were dried over MgSO₄, filtered and concentrated in vacuo. The residue was purified by prep. HPLC (column: Waters XBridge, 30×75 mm, 10 um, UV/MS, basic conditions) to give a white solid. LC-MS (2): t_{R}=0.95 min; [M+H]⁺: 303.29.

### Step 2: 2-(2-(difluoromethoxy)pyridin-4-yl)propan-2-amine

To an ice-cooled solution of tert-butyl (2-(2-(difluoromethoxy)pyridin-4-yl)propan-2-yl)carbamate (28 mg, 0.09 mmol, 1 eq) in DCM (5 mL), TFA (71 μL, 0.9 mmol, 10 eq) was added. The resulting mixture was stirred at 0° C. for 10 min then at rt overnight. The reaction mixture was concentrated in vacuo. The residue was partitioned between sat. aq. NaHCO₃ soln. (40 mL) and DCM (40 mL). The layers were separated. The aq. phase was extracted with DCM (2×40 mL). The comb. org. phases were washed with sat. aq. NaCl soln. (40 mL), dried over MgSO₄, and concentrated in vacuo. The residue was used crude for the next step. LC-MS (2): t_{R}=0.48 min; [M+H]⁺: 203.30.

The following amine was synthesized using the appropriate nitrile and Boc-protected amino acid derivatives and following the procedure described for 2-(2-(difluoromethoxy)pyridin-4-yl)propan-2-amine (Steps 1 and 2). LC-MS data are listed in the table below. The LC-MS conditions used were LC-MS (2).

| Name | t_{R} | [M + H]⁺ |
|---|---|---|
| 1-(2-(difluoromethoxy)pyridin-4-yl)cyclobutan-1- | 0.52 | 215.28 |
| amine | | |

### Synthesis of (±)-2-methoxy-N-methyl-1-(2-(2,2,2-trifluoroethoxy)pyridin-4-yl)ethan-1-amine trifluoroacetate

### Step 1: tert-butyl (±)-(2-methoxy-1-(2-(2,2,2-trifluoroethoxy)pyridin-4-yl)ethyl)(methyl)carbamate

To a solution of tert-butyl (±)-(2-methoxy-1-(2-(2,2,2-trifluoroethoxy)pyridin-4-yl)ethyl)carbamate (60 mg, 0.17 mmol, 1.0 eq) in DMF (3 mL), silver(I) oxide (156 mg, 0.67 mmol, 3.9 eq) and iodomethane (16 μL, 0.26 mmol, 1.5 eq) were added in sequence. The resulting mixture was heated at 50° C. overnight. Water was added and the mixture was extracted with EtOAc (3×). The comb. org. layers were dried over MgSO₄, filtered and concentrated in vacuo. The residue was used without further purification. LC-MS (2): t_{R}=1.03 min; [M+H]⁺: 365.23.

### Step 2: (±)-2-methoxy-N-methyl-1-(2-(2,2,2-trifluoroethoxy)pyridin-4-yl)ethan-1-amine trifluoroacetate

To an ice-cooled solution of tert-butyl (±)-(2-methoxy-1-(2-(2,2,2-trifluoroethoxy)pyridin-4-yl)ethyl)carbamate (54 mg, 0.15 mmol, 1 eq) in DCM (5 mL), trifluoroacetic acid (0.11 mL, 1.5 mmol, 10 eq) was added. The resulting solution was stirred at rt overnight. The reaction mixture was concentrated in vacuo. The residue was used without further purification. LC-MS (2): t_{R}=0.59 min; [M+H]⁺: 265.24.

### Synthesis of (3-(1-(trifluoromethyl)cyclopropyl)phenyl)methanamine

### Step 1: tert-butyl (3-(3,3,3-trifluoroprop-1-en-2-yl)benzyl)carbamate

A mixture of 3-(N-boc-aminomethyl)phenylboronic acid (400 mg, 1.55 mmol, 1.00 eq), 2-bromo-3,3,3-trifluoro-1-propene (0.19 mL, 1.82 mmol, 1.18 eq), Cs₂CO₃ (1.41 g, 4.33 mmol, 2.80 eq) and tetrakis(triphenylphosphine)palladium(0) (320 mg, 0.28 mmol, 0.18 eq) in DMF (10 mL) was degassed with N₂ for 5 min. Then, the mixture was heated at 70° C. overnight. The reaction mixture was allowed to cool to rt and was diluted with water, extracted with EtOAc, dried over MgSO₄, filtered and concentrated in vacuo. The residue was purified by prep. HPLC (column: Waters X-Bridge, 30×75 mm, 10 um, UV/MS, basic conditions) to yield a white solid. LC-MS (2): t_{R}=1.02 min; no ionization.

### Step 2: tert-butyl (3-(1-(trifluoromethyl)cyclopropyl)benzyl)carbamate

To a mixture of 2,4,5,6-tetra(9H-carbazol-9-yl)isophthalonitrile (5.9 mg, 7.5 μmol, 0.05 eq) and triethylamine-2-(iodomethyl)-2X⁵-2,2′-spirobi[benzo[d][1,3,2]dioxasilole] (109 mg, 0.224 mmol, 1.5 eq) in DMSO (2 mL), tert-butyl (3-(3,3,3-trifluoroprop-1-en-2-yl)benzyl)carbamate (45 mg, 0.149 mmol, 1 eq) was added. The resulting mixture was degassed with N₂ while stirring for 15 min. Then, the resulting mixture was stirred at rt for 5 hours under blue LED irradiation. Water was added and the mixture was extracted with EtOAc (3×). The comb. org. layers were dried over MgSO₄, filtered and concentrated in vacuo. The residue was purified by prep. HPLC (column: Waters X-Bridge, 30×75 mm, 10 um, UV/MS, basic conditions) to afford a brown oil. LC-MS (2): t_{R}=1.05 min; no ionization.

### Step 3: (3-(1-(trifluoromethyl)cyclopropyl)phenyl)methanamine

To an ice-cooled solution of tert-butyl (3-(1-(trifluoromethyl)cyclopropyl)benzyl)carbamate (25 mg, 0.08 mmol, 1 eq) in DCM (10 mL), TFA (61 μL, 0.79 mmol, 10 eq) was added. The resulting mixture was stirred at 0° C. for 10 min then at rt overnight. The reaction mixture was concentrated in vacuo. The residue was partitioned between sat. aq. NaHCO₃ soln. (40 mL) and DCM (40 mL). The layers were separated. The aq. phase was extracted with DCM (2×40 mL). The comb. org. phases were washed with sat. aq. NaCl soln. (40 mL), dried over MgSO₄, and concentrated in vacuo to give a colorless oil. The residue was used without further purification. LC-MS (2): t_{R}=0.62 min; [M+H]⁺: 216.26.

### Synthesis of 2-(aminomethyl)-4-(trifluoromethyl)phenol

To a solution of 2-hydroxy-5-(trifluoromethyl)benzaldehyde (400 mg, 2 mmol, 1 eq) in MeOH (40 mL), ammonium acetate (3.08 g, 40 mmol, 20 eq) and sodium cyanoborohydride (251 mg, 4 mmol, 2 eq) were added in sequence. The resulting mixture was stirred at rt overnight. The resulting mixture was concentrated in vacuo. The residue was partitioned between 2M aq. HCl soln. (50 mL) and EtOAc (50 mL). The layers were separated. The org. phase was extracted with 2M aq. HCl soln. (2×50 mL). The comb. 3 acidic aq. phases were basified with 25% NH₃ and extracted with DCM (5×60 mL). The comb. org. phases were washed with sat. aq. NaCl soln. (1×20 mL), dried over MgSO₄, filtered and concentrated in vacuo to give a white solid. The product was used without further purification. LC-MS (2): t_{R}=0.50 min; [M+H]⁺: 192.18.

### Synthesis of (±)-(3-fluoro-5-((1,1,1-trifluoropropan-2-yl)oxy)phenyl)methanamine

### Step 1: (±)-3-fluoro-5-((1,1,1-trifluoropropan-2-yl)oxy)benzonitrile

To an ice-cooled solution of 1,1,1-trifluoropropan-2-ol (627 mg, 5.5 mmol, 1.1 eq) in DMF (5 mL), sodium hydride 60% dispersion in mineral oil (260 mg, 6.5 mmol, 1.3 eq) was added. The mixture was stirred at 0° C. for 30 min. 3,5-Difluorobenzonitrile (703 mg, 5 mmol, 1 eq) was added dropwise. Upon addition completion, the cooling bath was removed and the mixture stirred at rt for 1.5 hours. The mixture was partitioned between water (25 mL) and Et₂O (25 mL). The layers were separated. The org. phase was washed with sat. aq. NaCl soln. (25 mL), dried over MgSO₄ and concentrated in vacuo. The residue was purified by Combiflash (column: 80 g, flow: 60 mL/min, heptane to heptane/EtOAc 100:10) to give a colorless oil. LC-MS (2): t_{R}=0.99 min; no ionization.

### Step 2: (±)-(3-fluoro-5-((1,1,1-trifluoropropan-2-yl)oxy)phenyl)methanamine

To an ice-cooled suspension of LiAlH₄ (288 mg, 7.59 mmol, 3 eq) in THF (10 mL), a solution of (±)-3-fluoro-5-((1,1,1-trifluoropropan-2-yl)oxy)benzonitrile (590 mg, 2.53 mmol, 1 eq) in THF (5 mL) was added dropwise. The mixture was stirred at 0° C. for 1 hour and further at rt for 1 hour. The reaction was cooled to 0° C. and quenched with iPrOH (2 mL) and 1N aq. NaOH soln. (15 mL). The resulting suspension was filtered through Celite. The filter cake was rinsed with EtOAc. The filtrate was partially concentrated in vacuo. The resulting aq. phase was diluted with 1M aq. NaOH soln. (20 mL) and extracted with DCM (3×20 mL). The comb. org. phases were washed with H₂O (1×20 mL), sat. aq. NaCl soln. (1×20 mL), dried over MgSO₄, and concentrated in vacuo to give a pale yellow oil. The product was used without further purification. LC-MS (2): t_{R}=0.62 min; [M+H]⁺: 238.12.

### Synthesis of (±)-1-(3-((1,1,1-trifluoropropan-2-yl)oxy)phenyl)cyclopropan-1-amine hydrochloride

### Step 1: (±)-3-((1,1,1-trifluoropropan-2-yl)oxy)benzonitrile

To an ice-cooled solution of 1,1,1-trifluoropropan-2-ol (627 mg, 5.5 mmol, 1.1 eq) in DMF (5 mL), sodium hydride 60% dispersion in mineral oil (260 mg, 6.5 mmol, 1.3 eq) was added. The mixture was stirred at 0° C. for 30 min. 3-Fluorobenzonitrile (618 mg, 5 mmol, 1 eq) was added dropwise. Upon addition completion, the cooling bath was removed and the mixture stirred at rt for 72 hours. The mixture was partitioned between water (25 mL) and Et₂O (25 mL). The layers were separated. The org. phase was washed with sat. aq. NaCl soln. (25 mL), dried over MgSO₄ and concentrated in vacuo. The residue was purified by Combiflash (column: 80 g, flow: 60 mL/min, heptane to heptane/EtOAc 100:15) to give a colorless oil. LC-MS (2): t_{R}=0.96 min; no ionization.

### Step 2: (±)-1-(3-((1,1,1-trifluoropropan-2-yl)oxy)phenyl)cyclopropan-1-amine hydrochloride

To a solution of (±)-3-((1,1,1-trifluoropropan-2-yl)oxy)benzonitrile (750 mg, 3.49 mmol, 1.0 eq) and titanium(IV)isopropoxide (1.15 mL, 3.83 mmol, 1.1 eq) in Et₂O (15 mL) cooled at −78° C., 3M ethylmagnesium bromide in ether (2.6 mL, 7.67 mmol, 2.2 eq) was added dropwise. The solution was stirred at −78° C. for 30 min, then allowed to warm to rt. Boron trifluoride etherate (0.92 mL, 6.97 mmol, 2.0 eq) was added and the mixture was stirred at rt for 1 hour. The mixture was diluted with Et₂O (35 mL) and 1N aq. HCl soln. (10 mL) was added. 10% aq. NaOH soln. (30 mL) was added to the biphasic mixture. The layers were separated and the aq. phase was extracted with Et₂O (2×50 mL). The comb. org. phases were dried over MgSO₄ and concentrated in vacuo. The residue was dissolved in 4M HCl soln. in dioxane (2 mL) and concentrated in vacuo to give a yellow oil that solidified upon standing. The product was used without further purification. LC-MS (2): t_{R}=0.65 min; [M+H]⁺: 246.19.

### Synthesis of (±)-1-(2-(difluoromethoxy)pyridin-4-yl)ethan-1-amine

### Step 1: 1-(2-(difluoromethoxy)pyridin-4-yl) ethan-1-one

To an ice-cooled solution of 2-(difluoromethoxy)pyridine-4-carbonitrile (450 mg, 2.51 mmol, 1 eq.0) in THE (20 mL), 3M methylmagnesium bromide in diethyl ether (1.84 mL, 5.53 mmol, 2.2 eq) was added dropwise. The resulting mixture was stirred at rt overnight. The resulting mixture was quenched with 10% acetic acid aq. soln. (15 mL). The reaction mixture was diluted with sat. aq. NaHCO₃ soln. (30 mL) and EtOAc (30 mL). The layers were separated and the aq. phase was extracted with EtOAc (1×30 mL). The comb. org. phases were washed with sat. aq. NaCl soln. (1×20 mL), dried over MgSO₄, and concentrated in vacuo. The residue was purified by Combiflash (column: 40 g, flow: 40 mL/min, heptane to heptane/EtOAc 80:20) to give an yellow oil. LC-MS (2): t_{R}=0.79 min; no ionization.

### Step 2: (±)-1-(2-(difluoromethoxy)pyridin-4-yl)ethan-1-amine

To a solution of 1-(2-(difluoromethoxy)pyridin-4-yl)ethan-1-one (170 mg, 0.91 mmol, 1.0 eq) in MeOH (20 mL), ammonium acetate (357 mg, 4.54 mmol, 5.0 eq) and sodium cyanoborohydride (90 mg, 1.36 mmol, 1.5 eq) were added in sequence. The resulting mixture was stirred at rt overnight. The reaction mixture was diluted with sat. aq. NaHCO₃ soln. (30 mL) and EtOAc (30 mL). The layers were separated and the aq. phase was extracted with EtOAc (1×30 mL). The comb. org. phases were washed with sat. aq. NaCl soln. (1×20 mL), dried over MgSO₄, and concentrated in vacuo. The residue was used without further purification. LC-MS (2): t_{R}=0.43 min; [M+H]⁺: 189.32.

The following amines were synthesized using the appropriate nitrile derivative and following the procedure described for (±)-1-(2-(difluoromethoxy)pyridin-4-yl)ethan-1-amine (Steps 1 and 2). LC-MS data are listed in the table below. The LC-MS conditions used were LC-MS (2).

| Name | t_{R} | [M + H]⁺ |
|---|---|---|
| 1-(2-((1,1,1-trifluoropropan-2-yl)oxy)pyridin-4- | 0.59 | 234.91 |
| yl)ethan-1-amine | | |
| 1-(3-((1,1,1-trifluoropropan-2-yl)oxy)phenyl)ethan- | 0.62 | 234.09 |
| 1-amine | | |
| (±)-1-(2-methyl-6-(2,2,2-trifluoroethoxy)pyrimidin- | 0.55 | 236.12 |
| 4-yl)ethan-1-amine | | |
| (±)-1-(5-methoxy-6-(2,2,2-trifluoroethoxy)pyrimidin- | 0.56 | 252.22 |
| 4-yl)ethan-1-amine | | |

### Synthesis of 4-(aminomethyl)-N-methyl-N-(2,2,2-trifluoroethyl)pyridin-2-amine

A solution of 2-(methyl(2,2,2-trifluoroethyl)amino)isonicotinonitrile (110 mg, 0.51 mmol, 1 eq) in MeOH (5 mL) was circulated in an HCube-Pro using a Raney Nickel cartridge (3 cm) at 80° C., 30 bar, and with a flow of 0.3 mL/min under 100% H₂ mode. The solvent was then removed in vacuo. The product was used without further purification. LC-MS (2): t_{R}=0.31 min; [M+H]⁺:220.20.

The following amines were synthesized using the appropriate nitrile derivative and following the procedure described for 4-(aminomethyl)-N-methyl-N-(2,2,2-trifluoroethyl)pyridin-2-amine. LC-MS data are listed in the table below. The LC-MS conditions used were LC-MS (2).

| Name | t_{R} | [M + H]⁺ |
|---|---|---|
| 4-(aminomethyl)-N-(2,2,2-trifluoroethyl)pyridin-2- | 0.21 | 206.29 |
| amine | | |
| (±)-(2-((1,1,1-trifluoropropan-2-yl)oxy)pyrimidin- | 0.45 | 222.22 |
| 4-yl)methanamine | | |
| (±)-(4-((1,1,1-trifluoropropan-2-yl)oxy)pyridin-2- | 0.49 | 221.24 |
| yl)methanamine | | |
| (±)-2-((6-(aminomethyl)pyridin-2-yl)oxy)-3,3,3- | 0.35 | 264.24 |
| trifluoro-N,N-dimethylpropan-1-amine | | |
| (±)-(6-((1,1,1-trifluoro-3-methoxypropan-2- | 0.58 | 251.23 |
| yl)oxy)pyridin-2-yl)methanamine | | |
| (±)-(2-((1,1,1-trifluoro-3-methoxypropan-2- | 0.54 | 251.18 |
| yl)oxy)pyridin-4-yl)methanamine | | |
| (±)-2-((4-(aminomethyl)pyridin-2-yl)oxy)-3,3,3- | 0.30 | 264.22 |
| trifluoro-N,N-dimethylpropan-1-amine | | |
| (3-((trifluoromethyl)sulfonyl)phenyl)methanamine | 0.54 | 239.98 |
| (±)-(6-((1,1,1-trifluoropropan-2-yl)oxy)pyrimidin- | 0.52 | 222.26 |
| 4-yl)methanamine | | |

### Synthesis of (6-(2,2,2-trifluoroethoxy)pyrimidin-4-yl)methanamine hydrochloride

### Step 1: 6-(2,2,2-trifluoroethoxy)pyrimidine-4-carbonitrile

To an ice-cooled solution of 2,2,2-trifluoroethanol (2.33 mL, 31.7 mmol, 1.2 eq) in THE (100 mL), sodium hydride 60% dispersion in mineral oil (1.37 g, 34.3 mmol, 1.3 eq) was added. The mixture was stirred at 0° C. for 10 min. Then 6-chloropyrimidine-4-carbonitrile (3.80 g, 26.4 mmol, 1.0 eq) was added. The resulting mixture was stirred at 0° C. for 20 min. The reaction mixture was partitioned between water and EtOAc (50 mL). The layers were separated. The org. phase was dried over MgSO₄ and concentrated in vacuo. The residue was used without further purification. LC-MS (2): t_{R}=0.82 min; [M+H]⁺: no ionization.

### Step 2: tert-butyl ((6-(2,2,2-trifluoroethoxy)pyrimidin-4-yl)methyl)carbamate

To an ice-cooled solution under N2 of 6-(2,2,2-trifluoroethoxy)pyrimidine-4-carbonitrile (5.50 g, 27.1 mmol, 1.0 eq) MeOH (100 mL), di-tert-butyl dicarbonate (12.06 g, 54.2 mmol, 2.0 eq) and nickel(II) chloride hexahydrate (1.31 g, 5.42 mmol, 0.2 eq) were added in sequence. Sodium borohydride (7.24 g, 190 mmol, 7.0 eq) was added in small portions over 10 min. The mixture was allowed to warm to rt and stirred at rt for 30 min. Diethylenetriamine (2.94 mL, 27.1 mmol, 1.0 eq) was added and the mixture was stirred at rt for 30 min. The solvent was evaporated. The resulting precipitate was taken up in EtOAc (100 mL) and washed with sat. aq. NaHCO₃ soln. (2×100 mL). The comb. aq. layers were extracted with EtOAc (1×50 mL). The comb. org. phases were dried over MgSO₄, filtered and evaporated. The residue was purified by Combiflash (column: 220 g, flow: 150 mL/min, Heptane to Heptane+50% EtOAc) to yield an yellow oil. LC-MS (2): t_{R}=0.90 min; [M+H]⁺: 308.20.

### Step 3: (6-(2,2,2-trifluoroethoxy)pyrimidin-4-yl)methanamine hydrochloride

To an ice-cooled solution of tert-butyl ((6-(2,2,2-trifluoroethoxy)pyrimidin-4-yl)methyl)carbamate (2.90 g, 9.44 mmol, 1 eq) in DCM (30 mL), 4M HCl in dioxane (4 mL) was added. The resulting mixture was stirred at 0° C. for 10 min and further at rt overnight. The reaction mixture was concentrated in vacuo. The residue was used without further purification. LC-MS (2): t_{R}=0.41 min; [M+H]⁺: 208.20.

The following amine salts were synthesized using the appropriate nitrile derivative and following the procedure described for (6-(2,2,2-trifluoroethoxy)pyrimidin-4-yl)methanamine hydrochloride (Steps 1 to 3). LC-MS data are listed in the table below. The LC-MS conditions used were LC-MS (2).

| Name | t_{R} | [M + H]⁺ |
|---|---|---|
| (2-methyl-6-(2,2,2-trifluoroethoxy)pyrimidin-4- | 0.50 | 222.23 |
| yl)methanamine hydrochloride | | |
| (2-(2,2,2-trifluoroethoxy)pyrimidin-4-yl)methanamine | 0.37 | 208.13 |
| hydrochloride | | |

### Synthesis of (±)-2-((1,1,1-trifluoropropan-2-yl)oxy)isonicotinonitrile

To an ice-cooled solution of 1,1,1-trifluoro-2-propanol (647 mg, 5.5 mmol, 1.1 eq) in THE (5 mL), sodium hydride 60% dispersion in mineral oil (260 mg, 6.5 mmol, 1.3 eq) was added. The mixture was stirred at 0° C. for 30 min. 2-Fluoroisonicotinonitrile (643 mg, 5 mmol, 1.0 eq) was added dropwise. Upon addition completion, the cooling bath was removed and the mixture stirred at rt overnight. The mixture was partitioned between water (25 mL) and Et₂O (25 mL). The layers were separated. The org. phase was washed with sat. aq. NaCl soln. (25 mL), dried over MgSO₄ and concentrated in vacuo. The residue was used without further purification. LC-MS (2): t_{R}=0.94 min; no ionization.

The following nitriles were synthesized using the appropriate fluoro or chloro derivatives and the appropriate alcohol derivatives and following the procedure described for (±)-2-((1,1,1-trifluoropropan-2-yl)oxy)isonicotinonitrile. LC-MS data are listed in the table below. The LC-MS conditions used were LC-MS (2).

| Name | t_{R} | [M + H]⁺ |
|---|---|---|
| 1-(2-((1,1,1-trifluoropropan-2-yl)oxy)pyridin-4- | 0.59 | 234.91 |
| yl)ethan-1-amine | | |
| (±)-3-((1,1,1-trifluoropropan-2- | 0.96 | No ionization |
| yl)oxy)benzonitrile | | |
| (±)-2-((1,1,1-trifluoropropan-2-yl)oxy)pyrimidine- | 0.86 | 218.16 |
| 4-carbonitrile | | |
| (±)-4-((1,1,1-trifluoropropan-2- | 0.86 | 217.20 |
| yl)oxy)picolinonitrile | | |
| (±)-6-((1,1,1-trifluoro-3-methoxypropan-2- | 0.92 | 247.17 |
| yl)oxy)picolinonitrile | | |
| (±)-6-((3-(dimethylamino)-1,1,1- | 0.55 | 260.18 |
| trifluoropropan-2-yl)oxy)picolinonitrile | | |
| 2-methyl-6-(2,2,2-trifluoroethoxy)pyrimidine-4- | 0.87 | No ionization |
| carbonitrile | | |
| (±)-6-((1,1,1-trifluoropropan-2-yl)oxy)pyrimidine- | 0.89 | No ionization |
| 4-carbonitrile | | |

### Synthesis of (±)-2-((3-(dimethylamino)-1,1,1-trifluoropropan-2-yl)oxy)isonicotinonitrile

To an ice-cooled solution of 3-(dimethylamino)-1,1,1-trifluoropropan-2-ol (425 mg, 2.57 mmol, 1.1 eq) in THE (5 mL), sodium hydride 60% dispersion in mineral oil (121 mg, 3.03 mmol, 1.3 eq) was added. The mixture was stirred at 0° C. for 30 min. 2-Fluoroisonicotinonitrile (300 mg, 2.33 mmol, 1 eq) was added dropwise. Upon addition completion, the cooling bath was removed and the mixture stirred at 60° C. overnight. The mixture was partitioned between water (25 mL) and Et₂O (25 mL). The layers were separated. The org. phase was washed with sat. aq. NaCl soln. (25 mL), dried over MgSO₄ and concentrated in vacuo. The residue was purified by Combiflash (column: 40 g, flow: 40 mL/min, Heptane to Heptane+50% EtOAc) to give a colorless oil. LC-MS (2): t_{R}=0.55 min; [M+H]⁺: 260.19. The following nitrile was synthesized using the appropriate fluoro derivatives and following the procedure described for (±)-2-((3-(dimethylamino)-1,1,1-trifluoropropan-2-yl)oxy)isonicotinonitrile. LC-MS data are listed in the table below. The LC-MS conditions used were LC-MS (2).

| Name | t_{R} | [M + H]⁺ |
|---|---|---|
| (±)-2-((1,1,1-trifluoro-3-methoxypropan-2- | 0.93 | 247.16 |
| yl)oxy)isonicotinonitrile | | |

### Synthesis of 2-(methyl(2,2,2-trifluoroethyl)amino)isonicotinonitrile

To a solution of 2-fluoroisonicotinonitrile (257 mg, 2.0 mmol, 1.0 eq) in NMP (3 mL), methyl-(2,2,2-trifluoro-ethyl)-amine hydrochloride (329 mg, 2.2 mmol, 1.1 eq) and NEt₃ (0.61 mL, 4.4 mmol, 2.2 eq) were added in sequence. The reaction mixture was stirred at 170° C. under microwave irradiation for 5 hours. The reaction mixture was purified by prep. HPLC (column: Waters XBridge, 30×75 mm, 10 um, UV/MS, basic conditions). LC-MS (2): t_{R}=0.89 min; [M+H]⁺: 216.24.

The following nitrile was synthesized using the appropriate fluoro derivatives and following the procedure described for 2-(methyl(2,2,2-trifluoroethyl)amino)isonicotinonitrile. LC-MS data are listed in the table below. The LC-MS conditions used were LC-MS (2).

| Name | t_{R} | [M + H]⁺ |
|---|---|---|
| 2-((2,2,2-trifluoroethyl)amino)isonicotinonitrile | 0.79 | 202.27 |

### Synthesis of 5-methoxy-6-(2,2,2-trifluoroethoxy)pyrimidine-4-carbonitrile

### Step 1: 4-chloro-5-methoxy-6-(2,2,2-trifluoroethoxy)pyrimidine

To a solution of 2,2,2-trifluoroethanol (0.22 mL, 3.01 mmol, 1.1 eq) in THF (3 mL), sodium hydride 60% dispersion in mineral oil (120 mg, 3.01 mmol, 1.1 eq) was added. The resulting mixture was stirred at rt for 15 min. Then a solution of 4,6-dichloro-5-methoxypyrimidine (500 mg, 2.74 mmol, 1.0 eq) in THF (2 mL) was added. The resulting mixture was stirred at 45° C. for 2 h. The reaction mixture was quenched with sat. aq. NH₄Cl soln. (30 mL) and extracted with EtOAc (3×). The comb. org. phases were dried over MgSO₄, filtered and concentrated in vacuo. The crude was purified by flash chromatography (heptane/EtOAc 9:1) to afford a white solid. LC-MS (2): t_{R}=0.89 min; [M+H]⁺: 242.99.

### Step 2: 5-methoxy-6-(2,2,2-trifluoroethoxy)pyrimidine-4-carbonitrile

A mixture of 4-chloro-5-methoxy-6-(2,2,2-trifluoroethoxy)pyrimidine (460 mg, 1.9 mmol, 1.00 eq), zinc cyanide (250 mg, 2.1 mmol, 1.10 eq), tris(dibenzylideneacetone)dipalladium(0) (52 mg, 0.06 mmol, 0.03 eq) and 1,1′-bis(diphenylphosphino)ferrocene (64 mg, 0.11 mmol, 0.06 eq) in DMF (6 mL) was degassed with a nitrogen stream for 10 min. The mixture was then stirred at 110° C. overnight. The mixture was allowed to cool to rt, diluted with water and extracted with EtOAc (3×). The comb. org. layers were dried over MgSO₄ and concentrated in vacuo. The residue was purified by Combiflash (column: 12 g, flow: 25 mL/min, Heptane to Heptane+20% EtOAc) to yield a light brown oil. LC-MS (2): t_{R}=0.86 min; [M+H]⁺: 234.11.

### Synthesis of (3-((trifluoromethoxy)methyl)phenyl)methanamine

### Step 1: tert-butyl (3-((trifluoromethoxy)methyl)benzyl)carbamate

To a solution of tert-butyl 3-(hydroxymethyl)benzylcarbamate (237 mg, 1.0 mmol, 1.0 eq) in EtOAc (5 mL), potassium fluoride (246 mg, 4.2 mmol, 4.2 eq), silver trifluoromethanesulfonate (779 mg, 3.0 mmol, 3.0 eq), selectfluor (548 mg, 1.5 mmol, 1.5 eq), 2-fluoropyridine (0.26 mL, 3.0 mmol, 3.0 eq) and trimethyl(trifluoromethyl)silane (0.45 mL, 3.0 mmol, 3.0 eq) were added in sequence. The resulting mixture was degassed with N₂ while stirring for 15 minutes. Then, the resulting mixture was stirred at rt overnight. Water was added and the mixture was extracted with EtOAc (3×). The comb. org. layers were dried over MgSO₄, filtered and concentrated in vacuo. The residue was purified by prep. HPLC (column: Waters XBridge, 30×75 mm, 10 um, UV/MS, basic conditions). LC-MS (2): t_{R}=1.01 min; no ionization.

### Step 2: (3-((trifluoromethoxy)methyl)phenyl)methanamine

To an ice-cooled solution of tert-butyl (3-((trifluoromethoxy)methyl)benzyl)carbamate (36 mg, 0.12 mmol, 1 eq) in DCM (10 mL), TFA (90 μL, 1.18 mmol, 10 eq) was added. The resulting mixture was stirred at 0° C. for 10 min then at rt overnight. The reaction mixture was concentrated in vacuo. The residue was partitioned between sat. aq. NaHCO₃ (40 mL) and DCM (40 mL). The layers were separated. The aq. phase was extracted with DCM (2×40 mL). The comb. org. phases were washed with sat. aq. NaCl soln. (40 mL), dried over MgSO₄, and concentrated in vacuo. The residue was used without further purification. LC-MS (2): t_{R}=0.57 min; [M+H]⁺: 206.26.

### Synthesis of 3-(3-(trifluoromethyl)phenyl)oxetan-3-amine hydrochloride

### Step 1: (±)-2-methyl-N-(oxetan-3-ylidene)propane-2-sulfinamide

To a solution of 3-oxetanone (288 mg, 4.0 mmol, 1.0 eq) in THF (10 mL), (±)-2-methylpropane-2-sulfinamide (665 mg, 5.2 mmol, 1.3 eq) was added at rt. Titanium(IV) ethoxide (0.96 mL, 4.6 mmol, 1.1 eq) was then added dropwise and the resulting mixture was stirred at rt for 1 h30. Water (30 mL) and DCM (15 mL) were added and the biphasic mixture was filtered through celite. The layers of the filtrate were separated and the aq. phase was extracted with DCM (2×15 mL). The comb. org. phases were washed with water, sat. aq. NaCl soln., dried over MgSO₄, filtered and concentrated in vacuo to give a pale yellow liquid. The product was used without further purification. LC-MS (2): t_{R}=0.57 min; [M+H]⁺: 176.35.

### Step 2: (±)-2-methyl-N-(3-(3-(trifluoromethyl)phenyl)oxetan-3-yl)propane-2-sulfinamide

To a solution of 3-bromobenzotrifluoride (0.31 mL, 2.14 mmol, 1.5 eq) in THF (10 mL) cooled at −78° C., 2.5M n-butyllithium solution in hexanes (0.8 mL, 1.99 mmol, 1.4 eq) was added dropwise. The solution was stirred at −78° C. for 30 min. A solution of (±)-2-methyl-N-(oxetan-3-ylidene)propane-2-sulfinamide (320 mg, 1.42 mmol, 1.0 eq) in THF (2.5 mL) was then added dropwise and the resulting mixture was stirred at −78° C. for 10 min then was allowed to warm up to rt and was stirred at rt for 10 min. The reaction was quenched with sat. aq. NH₄Cl soln. (6 mL) and the mixture was partitioned between water (13 mL) and EtOAc (25 mL). The layers were separated and the aq. phase was extracted with EtOAc (2×25 mL). The comb. org. phases were washed with sat. aq. NaCl soln, dried over MgSO₄, filtered and concentrated in vacuo. The residue was purified by Combiflash (column: 12 g, flow: 30 mL/min, Heptane+20% EtOAc to EtOAc 100%) to yield a colorless oil. LC-MS (2): t_{R}=0.85 min; [M+H]⁺: 321.97.

### Step 3: 3-(3-(trifluoromethyl)phenyl)oxetan-3-amine hydrochloride

To an ice-cooled solution of (±)-2-methyl-N-(3-(3-(trifluoromethyl)phenyl)oxetan-3-yl)propane-2-sulfinamide (75 mg, 0.23 mmol, 1.0 eq) in MeOH (0.8 mL), 4M HCl in dioxane (0.09 mL, 0.35 mmol, 1.5 eq) was added. The mixture was stirred at 0° C. for 5 min. The solvent was removed in vacuo to give a white-off solid. The product was used without further purification. LC-MS (2): t_{R}=0.52 min; [M+H]⁺: 218.23.

### Synthesis of (±)-1-amino-2-methyl-1-(3-(trifluoromethyl)phenyl)propan-2-ol

### Step 1: methyl (±)-2-(2,2,2-trifluoroacetamido)-2-(3-(trifluoromethyl)phenyl)acetate

To a solution of methyl 2-amino-2-[3-(trifluoromethyl)phenyl]acetate hydrochloride (568 mg, 2.0 mmol, 1.0 eq) and DIPEA (0.43 mL, 2.5 mmol, 1.25 eq) in DCM (3 mL), trifluoroacetic anhydride (0.34 mL, 2.4 mmol, 1.2 eq) was added. The resulting solution was stirred at rt for 1 h. The reaction was quenched with 1M aq. HCl soln. (3 mL) and DCM (7.5 mL) was added. The layers were separated and the org. phase was washed with sat. aq. NaHCO₃ soln., sat. aq. NaCl soln., dried over MgSO₄, filtered and concentrated in vacuo to give a pale orange oil that solidified upon standing. The residue was used without further purification. LC-MS (2): t_{R}=0.96 min; no ionization.

### Step 2: (±)-2,2,2-trifluoro-N-(2-hydroxy-2-methyl-1-(3-(trifluoromethyl) phen yl) propyl) acetamide

To a solution of methyl (±)-2-(2,2,2-trifluoroacetamido)-2-(3-(trifluoromethyl)phenyl)acetate (668 mg, 2.0 mmol, 1 eq) in THE (10 mL) at 0° C., 3M methylmagnesium bromide solution in diethyl ether (6.7 mL, 20.1 mmol, 10 eq) was added dropwise. The resulting mixture was stirred at 0° C. for 2 h30. The reaction was quenched by the addition of sat. aq. NH₄Cl soln. (12 mL). Water and DCM were added and the layers were separated. The aq. phase was extracted with DCM (2×) and the comb. org. layers were dried over MgSO₄, filtered and concentrated in vacuo to give a green oil. The residue was used without further purification. LC-MS (2): t_{R}=0.93 min; [M+H]⁺: 330.07.

### Step 3: (±)-1-amino-2-methyl-1-(3-(trifluoromethyl)phenyl)propan-2-ol

To a solution of (±)-2,2,2-trifluoro-N-(2-hydroxy-2-methyl-1-(3-(trifluoromethyl)phenyl)propyl)acetamide (610 mg, 1.7 mmol, 1 eq) in MeOH (5 mL) and H₂O (0.5 mL), KOH (199 mg, 3.6 mmol, 2.1 eq) was added. The resulting mixture was heated up to 50° C. and was stirred at that temperature overnight. The mixture was allowed to cool to rt and concentrated in vacuo. The residue was dissolved in DCM and water was added. The layers were separated and the aq. phase was extracted with DCM (2×). The comb. org. phases were dried over MgSO₄, filtered and concentrated in vacuo to give a pale green oil. The residue was used without further purification. LC-MS (2): t_{R}=0.55 min; [M+H]⁺: 234.25.

### Synthesis of 5-(2,2,2-trifluoroethoxy)-1,2,3,4-tetrahydro-2,6-naphthyridine hydrochloride

### Step 1: 6-benzyl-5,6,7,8-tetrahydro-2,6-naphthyridin-1-ol

To a solution of 2,6-naphthyridin-1(2H)-one (3.50 g, 22.8 mmol, 1.0 eq) in MeCN (227 mL), benzyl bromide (3.04 mL, 25 mmol, 1.1 eq) was added. The beige suspension was stirred at 80° C. for 2.5 h. The reaction mixture was allowed to cool down to rt, and sodium triacetoxyborohydride (12.4 g, 56.9 mmol, 2.5 eq) was added. The reaction mixture was stirred at rt for 0.5 h. The reaction mixture was filtered and the filtrate was concentrated in vacuo. The residue was partitioned between and EtOAc. The layers were separated. The aq. phase was cooled to 0° C. and basified with 24% aq. NaOH soln. until pH=9-10. The mixture was stirred at 0° C. for 15 min. The resulting suspension was filtered to give a white solid. The product was used without further purification. LC-MS (2): t_{R}=0.43 min; [M+H]⁺: 241.26.

### Step 2: 2-benzyl-5-(2,2,2-trifluoroethoxy)-1, 2, 3, 4-tetrahydro-2,6-naphthyridine

To an ice-cooled solution of 6-benzyl-5,6,7,8-tetrahydro-2,6-naphthyridin-1-ol (1.49 g, 6.18 mmol, 1.0 eq) in DMF (30 mL), sodium hydride, 60% dispersion in mineral oil (322 mg, 8.04 mmol, 1.3 eq) was added. The mixture was stirred at 0° C. for 15 min and further at rt for 15 min. The reaction mixture was then cooled to 0° C., 2,2,2-trifluoroethyl trifluoromethanesulfonate (1.18 mL, 8.04 mmol, 1.3 eq) was added dropwise and the resulting mixture was slowly warmed to rt and stirred at rt for 2 hours. The reaction mixture was quenched with water and extracted with EtOAc (3×). The comb. org. phases were washed with sat. aq. NaCl soln., dried over MgSO₄ and concentrated in vacuo. The residue was purified by Combiflash (column: 12 g, flow: 30 mL/min, Heptane+20% EtOAc to Heptane+50% EtOAc) to yield a light yellow oil. LC-MS (2): t_{R}=0.71 min; [M+H]⁺: 323.11. A second fraction containing 6-benzyl-2-(2,2,2-trifluoroethyl)-5,6,7,8-tetrahydro-2,6-naphthyridin-1(21-one was also isolated

### Step 3: 5-(2,2,2-trifluoroethoxy)-1, 2, 3, 4-tetrahydro-2,6-naphthyridine hydrochloride

To a solution of 2-benzyl-5-(2,2,2-trifluoroethoxy)-1,2,3,4-tetrahydro-2,6-naphthyridine (438 mg, 1.36 mmol, 1 eq) in toluene (11 mL), 1-chloroethyl chloroformate (0.3 mL, 2.72 mmol, 2 eq) was added. The mixture was stirred at rt for 10 min, then was heated up to 110° C. and stirred at that temperature for 1.5 h. The mixture was allowed to cool down to rt and 1-chloroethyl chloroformate (0.3 mL, 2.72 mmol, 2 eq) was added. The reaction mixture was further stirred at rt for 10 min then heated to 110° C. and stirred at that temperature for 1 h. The mixture was allowed to cool down to rt and was concentrated in vacuo. The residue was taken up in MeOH (4 mL) and the mixture was heated up to 70° C. and stirred at that temperature for 3 h. The mixture was allowed to cool down to rt, was concentrated under reduced pressure. The residue was dissolved in 4M HCl in dioxane (3.4 mL, 13.6 mmol, 10 eq) and stirred for 30 min. The white precipitate was recovered by filtration and dried under HV to afford a white solid. The product was used without further purification. LC-MS (2): t_{R}=0.53 min; [M+H]⁺: 233.20.

### Synthesis of (±)-1-(methoxymethyl)-5-(2,2,2-trifluoroethoxy)-1,2,3,4-tetrahydroisoquinoline

### Step 1: (E)-1-(2-nitrovinyl)-2-(2,2,2-trifluoroethoxy)benzene

A mixture of 2-(2,2,2-trifluoroethoxy)benzaldehyde (500 mg, 2.33 mmol, 1.0 eq) and ammonium acetate (201 mg, 2.56 mmol, 1.1 eq) in nitromethane (2.9 mL, 52 mmol, 22.4 eq) was stirred at 95° C. for 30 min. The reaction mixture was evaporated to dryness and the residue was partitioned between water and DCM. The aqueous phase was extracted with DCM (2×) and the comb. org. phases were washed with sat. aq. NaCl soln., dried over MgSO₄, filtered and concentrated in vacuo. The residue was purified by Combiflash (column: 12 g, flow: 25 mL/min, Heptane+30% EtOAc) to afford a light yellow solid. LC-MS (2): t_{R}=0.98 min; [M+H]⁺: no ionization.

### Step 2: 2-(2-(2,2,2-trifluoroethoxy)phenyl)ethan-1-amine

To an ice-cooled solution under N₂ of (E)-1-(2-nitrovinyl)-2-(2,2,2-trifluoroethoxy)benzene (325 mg, 1.3 mmol, 1.0 eq) in THF (11 mL), 2M LiAlH₄ in THF (2.3 mL, 4.6 mmol, 3.5 eq) was added dropwise over 5 min. The reaction mixture was stirred at 0° C. for 2 h and then slowly warmed to rt and further stirred at rt for 3 h. The reaction mixture was cooled to 0° C. and carefully quenched with water (0.18 mL), 15% aq. NaOH soln. (0.18 mL), and water (0.53 mL). The resulting suspension was stirred at rt overnight. The resulting red precipitate was filtered and washed with THF. The filtrate was dried over MgSO₄, and concentrated in vacuo to afford a light orange semi-solid The product was used without further purification. LC-MS (2): t_{R}=0.58 min; [M+H]⁺: 220.17.

### Step 3: 2-methoxy-N-(2-(2,2,2-trifluoroethoxy)phenethyl)acetamide

To an ice-cooled solution of 2-(2-(2,2,2-trifluoroethoxy)phenyl)ethan-1-amine (305 mg, 0.84 mmol, 1 eq) in DCM (8 mL), DIPEA (0.44 mL, 2.5 mmol, 3 eq) and a solution of methoxyacetyl chloride (79 μL, 0.84 mmol, 1 eq) in DCM (6.2 mL) were added in sequence. The resulting mixture was stirred at rt for 1 h. The reaction mixture was partitioned between DCM and water. The layers were separated and the aq. phase was extracted with DCM. The comb. org. phases were washed with water, sat. aq. NaCl soln., dried over MgSO₄ and concentrated in vacuo. The residue was purified by prep. HPLC (column: Waters XBridge, 30×75 mm, 10 um, UV/MS, basic conditions). LC-MS (2): t_{R}=0.96 min; [M+H]⁺: 292.26.

### Step 4: 1-(methoxymethyl)-5-(2,2,2-trifluoroethoxy)-3,4-dihydroisoquinoline

To a solution of 2-methoxy-N-(2-(2,2,2-trifluoroethoxy)phenethyl)acetamide (150 mg, 0.52 mmol, 1.0 eq) in phosphorus(V) oxychloride (0.24 mL, 2.57 mmol, 5 eq), phosphorus pentoxide (39 μL, 0.31 mmol, 0.6 eq) was added. The reaction mixture was stirred at 105° C. for 2 h, further at rt overnight, and at 105° C. for 5 h. The reaction mixture was allowed to cool down to rt and poured onto crushed ice. The resulting mixture was basified with 2M aq. NaOH soln. and extracted twice with DCM. The comb. org. phases were concentrated in vacuo. The residue was purified by prep. HPLC (column: Waters XBridge, 30×75 mm, 10 um, UV/MS, basic conditions). LC-MS (2): t_{R}=0.61 min; [M+H]⁺: 274.08.

### Step 5: (±)-1-(methoxymethyl)-5-(2,2,2-trifluoroethoxy)-1,2,3,4-tetrahydroisoquinoline

To an ice-cooled solution of 1-(methoxymethyl)-5-(2,2,2-trifluoroethoxy)-3,4-dihydroisoquinoline (115 mg, 0.34 mmol, 1.0 eq) in MeOH (5 mL), sodium borohydride (15 mg, 0.40 mmol, 1.2 eq) was added portionwise. The mixture was stirred at 0° C. for 10 min. The reaction was quenched with water and washed with DCM. The aq. layer was concentrated to afford an off-white solid. The residue was used without further purification. LC-MS (2): t_{R}=0.63 min; [M+H]⁺: 276.18.

### Synthesis of (±)-2-amino-N-methyl-2-(3-(trifluoromethyl)phenyl)acetamide trifluoroacetate

### Step 1: tert-butyl (±)-(2-(methylamino)-2-oxo-1-(3-(trifluoromethyl)phenyl)ethyl)carbamate

To a solution of N-boc-2-(3-trifluoromethyl-phenyl)-DL-glycine (64 mg, 0.20 mmol, 1.0 eq) and methylamine hydrochloride (15 mg, 0.20 mmol, 1.0 eq) in DMF (1.5 mL), DIPEA (0.17 mL, 1 mmol, 5.0 eq) and HATU (84 mg, 0.22 mmol, 1.1 eq) were added. The resulting solution was stirred at rt overnight. The crude mixture was purified by prep. HPLC (column: Waters XBridge, 30×75 mm, 10 um, UV/MS, basic conditions). LC-MS (2): t_{R}=0.88 min; [M+H]⁺: 333.29.

### Step 2: (±)-2-amino-N-methyl-2-(3-(trifluoromethyl)phenyl)acetamide trifluoroacetate

To an ice-cooled solution of tert-butyl (±)-(2-(methylamino)-2-oxo-1-(3-(trifluoromethyl)phenyl)ethyl)carbamate (67 mg, 0.2 mmol, 1 eq) in DCM (5 mL), trifluoroacetic acid (0.15 mL, 2 mmol, 10 eq) was added. The resulting solution was stirred at rt overnight. The reaction mixture was concentrated in vacuo. The residue was used without further purification. LC-MS (2): t_{R}=0.50 min; [M+H]⁺: 233.23.

### Synthesis of 2-(methylsulfonyl)-1-(3-(trifluoromethoxy)phenyl)ethan-1-amine

### Step 1: 2-(methylsulfonyl)-1-(3-(trifluoromethoxy)phenyl)ethan-1-one

To a solution under N₂ of 3-(trifluoromethoxy)phenacyl bromide (50 mg, 0.17 mmol, 1.0 eq) in dioxane (0.3 mL) and H₂O (0.3 mL) was added sodium methanesulfinate (26 mg, 0.25 mmol, 1.5 eq) at rt. The reaction mixture was heated at 100° C. for 2.5 h. The reaction mixture was evaporated. The residue was dissolved in DCM (5 mL), washed with H₂O (5 mL). The aq. phase was extracted with DCM (2×5 mL). The comb. org. phases were washed with sat. aq. NaCl soln. (10 mL), dried over Na₂SO₄, and concentrated in vacuo. The residue was purified by Combiflash (column: 4 g, flow: 15 mL/min, Heptane to Heptane+50% (TBME/MeOH 8:2)) to yield an yellow oil. LC-MS (2): t_{R}=0.82 min; no ionization.

### Step 2: 2-(methylsulfonyl)-1-(3-(trifluoromethoxy)phenyl)ethan-1-amine

To a solution of 2-(methylsulfonyl)-1-(3-(trifluoromethoxy)phenyl)ethan-1-one (45 mg, 0.15 mmol, 1 eq) in MeOH (10 mL), ammonium acetate (234 mg, 3 mmol, 20 eq) and sodium cyanoborohydride (20 mg, 0.3 mmol, 2 eq) were added in sequence. The resulting mixture was stirred at 40° C. for 24 h and further 60° C. for 5 days. The resulting mixture was concentrated in vacuo. The residue was diluted with sat. aq. NaHCO₃ soln. (5 mL) and DCM (5 mL). The layers were separated and the aq. phase was extracted with DCM (1×5 mL). The comb. org. phases were washed with sat. aq. NaCl soln. (1×5 mL), dried over Na₂SO₄, and concentrated in vacuo. The crude mixture was purified by prep. HPLC (column: Waters XBridge, 30×75 mm, 10 um, UV/MS, basic conditions). LC-MS (2): t_{R}=0.74 min; [M+H]⁺: 284.11.

### Synthesis of (±)-1-(2,2-difluorobenzo[d][1,3]dioxol-5-yl)ethan-1-amine

To a solution of 5-acetyl-2,3-dihydrobenzo(b)furan (502 mg, 3 mmol, 1 eq) in MeOH (100 mL), ammonium acetate (4.63 g, 60 mmol, 20 eq) and sodium cyanoborohydride (566 mg, 9 mmol, 3 eq) were added in sequence. The resulting mixture was stirred at 60° C. overnight. The resulting mixture was concentrated in vacuo. The residue was diluted with sat. aq. NaHCO₃ soln. and DCM. The layers were separated and the aq. phase was extracted with DCM (1×30 mL). The comb. org. phases were washed with sat. aq. NaCl soln. (1×20 mL), dried over MgSO₄, and concentrated in vacuo. The residue was used without further purification. LC-MS (2): t_{R}=0.54 min; [M+H]⁺: 202.28.

The following amines were synthesized using the appropriate ketone derivatives and following the procedure described for (±)-1-(2,2-difluorobenzo[d][1,3]dioxol-5-yl)ethan-1-amine. LC-MS data are listed in the table below. The LC-MS conditions used were LC-MS (2).

| Name | t_{R} | [M + H]⁺ |
|---|---|---|
| (±)-3-amino-3-(3- | 0.56 | 231.07 |
| (trifluoromethoxy)phenyl)propanenitrile | | |
| (±)-3-amino-3-(3- | 0.52 | 215.10 |
| (trifluoromethyl)phenyl)propanenitrile | | |

### Synthesis of 3-oxo-3-(3-(trifluoromethoxy)phenyl)propanenitrile

To an ice-cooled solution under N₂ of 3-(trifluoromethoxy)phenacyl bromide (250 mg, 0.84 mmol, 1 eq) in EtOH (3.5 mL), a solution of potassium cyanide (169 mg, 2.52 mmol, 3 eq) in H₂O (1 mL) was added. The reaction mixture was stirred at rt for 3 h. The reaction mixture was concentrated in vacuo. The residue was diluted with DCM (50 mL) and washed with sat. aq. NaHCO₃ soln. (50 mL). The layers were separated and the aq. phase was extracted with DCM (2×50 mL). The comb. org. phases were dried over Na₂SO₄, filtered and concentrated in vacuo. The residue was purified by Combiflash (column: 12 g, flow: 25 mL/min, Heptane+45% TBME) to afford a light yellow solid. LC-MS (2): t_{R}=0.88 min; no ionization.

The following nitrile was synthesized using the appropriate bromide derivatives and following the procedure described for 3-oxo-3-(3-(trifluoromethoxy)phenyl)propanenitrile. LC-MS data are listed in the table below. The LC-MS conditions used were LC-MS (2).

| Name | t_{R} | [M + H]⁺ |
|---|---|---|
| 3-oxo-3-(3-(trifluoromethyl)phenyl)propanenitrile | 0.85 | No ionization |

### Synthesis of (±)-2-(methylsulfonyl)-1-(3-(trifluoromethyl)phenyl)ethan-1-amine formate

### Step 1: 2-(methylsulfonyl)-1-(3-(trifluoromethyl)phenyl)ethan-1-one

To a solution under N₂ of 3-(trifluoromethoxy)phenacyl bromide (250 mg, 0.84 mmol, 1.0 eq) in dioxane (1.5 mL) and H₂O (1.5 mL), sodium methanesulfinate (128 mg, 1.26 mmol, 1.5 eq) was added at rt. The reaction mixture was heated at reflux for 1.5 h. The reaction mixture was evaporated. The residue was dissolved in DCM (25 mL), washed with H₂O (25 mL). The aq. phase was extracted with DCM (2×25 mL). The comb. org. phases were washed with sat. aq. NaCl soln. (50 mL), dried over Na₂SO₄, filtered and evaporated. The residue was purified by Combiflash (column: 12 g, flow: 25 mL/min, Heptane+20% TBME/MeOH 8:2) to afford a beige solid. LC-MS (2): t_{R}=0.78 min; no ionization.

### Step 2: (±)-2-(methylsulfonyl)-1-(3-(trifluoromethyl)phenyl)ethan-1-amine formate

To a solution of 2-(methylsulfonyl)-1-(3-(trifluoromethyl)phenyl)ethan-1-one (235 mg, 0.88 mmol, 1 eq) in MeOH (25 mL), ammonium acetate (1.36 g, 17.7 mmol, 20 eq) and sodium cyanoborohydride (117 mg, 1.77 mmol, 2 eq) were added in sequence. The resulting mixture was heated at 60° C. for 4 days. The resulting mixture was concentrated in vacuo. The residue was diluted with sat. aq. NaHCO₃ soln. (25 mL) and DCM (25 mL). The layers were separated and the aq. phase was extracted with DCM (25 mL). The comb. org. phases were washed with sat. aq. NaCl soln. (25 mL), dried over Na₂SO₄, and concentrated in vacuo. The crude mixture was purified by prep. HPLC (column: Waters XBridge, 30×75 mm, 10 um, UV/MS, acidic conditions). LC-MS (2): t_{R}=0.49 min; [M+H]⁺: 268.08.

### Synthesis of 2-(2-methoxyethoxy)-1-(3-(trifluoromethyl)phenyl)ethan-1-amine

To an ice-cooled solution of 2-amino-2-(3-trifluoromethyl-phenyl)-ethanol (50 mg, 0.24 mmol, 1.0 eq) in DMF (0.5 mL), sodium hydride 60% dispersion in mineral oil (12 mg, 0.29 mmol, 1.2 eq) was added. The reaction mixture was stirred at 0° C. for 45 min. 2-Bromoethyl methyl ether (34 μL, 0.37 mmol, 1.5 eq) was added. The reaction mixture was stirred at rt overnight. The reaction mixture was partitioned between EtOAc (5 mL) and H₂O (5 mL). The aq. phase was extracted with EtOAc (5 mL). The comb. org. phases were dried over Na₂SO₄, filtered and evaporated. The crude mixture was purified by prep. HPLC (column: Waters XBridge, 30×75 mm, 10 um, UV/MS, basic conditions). LC-MS (2): t_{R}=0.62 min; [M+H]⁺: 264.13.

### Synthesis of (±)-1-(3-(difluoromethoxy)phenyl)ethan-1-amine hydrochloride

To a solution of 3-(difluoromethoxy)benzonitrile (1.0 g, 5.79 mmol, 1 eq) in THE (5 mL), 3.4M methylmagnesium bromide solution in 2-methyltetrahydrofuran (5.11 mL, 17.4 mmol, 3 eq) was added. The mixture was stirred at rt for 2 hours. The reaction was cooled down to 15° C. and quenched with MeOH (20 mL). NaBH₄ (438 mg, 11.6 mmol, 2 eq) was added and the mixture was stirred at rt overnight. 2M aq. HCl soln. (30 mL) was added and the mixture was stirred at rt for 5 min. The organic solvent was evaporated in vacuo. The solution was diluted with DCM (50 mL) and washed with sat. aq. NaHCO₃ soln. (30 mL). The layers were separated. The org. phase was concentrated in vacuo. The residue was triturated with 1.25M HCl in MeOH and the suspension was concentrated in vacuo. The residue was used without further purification. LC-MS (3): t_{R}=0.73 min; [M+H]⁺: 188.34.

Determination of Absolute Stereochemistry

By slow diffusion of heptane into a concentrated solution of Example 190, 1-[(S)-1-(2-bromo-5-trifluoromethyl-phenyl)-ethyl]-3-spiro[3.3]hept-2-yl-urea (more active than the (R) enantiomer) in CHCl₃, suitable single crystals were obtained to carry out an X-ray crystal structure analysis and determine its absolute stereochemistry. The stereochemistry of other examples containing a substituent at the benzylic position which were obtained by chiral chromatography has been assigned in analogy, meaning that the more active isomer was assumed to have the same stereochemistry than Example 190.

II. Biological Assays

A) Rat Oscillation Assay:

Assay Principle

This assay is a functional phenotypic assay designed to mimic epileptic seizures using primary neuronal cultures from embryonic rat brains, which form a functional neuronal network that generate synchronized intracellular calcium concentration oscillations when cultured at high density in 384-well plate. The epileptic phenotype is induced by incubating the neurons in magnesium-free assay buffer, that results in increased probability of NMDA receptor activation, leading to an increased frequency and amplitude of intracellular calcium oscillations. Once neurons are incubated with the calcium indicator dye Fluo-8 AM (Tebu-bio), neuronal calcium oscillations can be monitored in real time using FLIPR® Tetra (fluorometric plate reader, Molecular Devices). With these recordings, the effect of anti-epileptic drugs can be quantified. The anti-epileptic effect of compounds, which activate directly or indirectly the Kᵥ7 channels can be modulated by the Kᵥ7 channel blocker XE-991. The assay was performed as described previously (Pacico N, Mingorance-Le Meur A. New In Vitro Phenotypic Assay for Epilepsy: Fluorescent Measurement of Synchronized Neuronal Calcium Oscillations. PLoS ONE 9(1) 2014) with modifications described hereafter.

Neuronal Cultures

Animal care followed standard procedures in accordance with swiss institutional guidelines. Dissociated neuronal cultures were obtained from cerebral cortices of embryonic Wistar rats at embryonic stage E18 (Charles River). The uterine horns were removed by caesarian surgery from deeply anesthetized rats (Isofurane) and sacrificed by decapitation. The embryos were decapitated by closing forceps. The brains were isolated and dissected one by one in ice-cold PBS (Life Technologies) under optical control using a binocular. Meninges, olfactory bulbs, and basal ganglia were removed. Cortical hemispheres (still including the hippocampus) were cut in small pieces with tweezers and placed on ice in pre-chilled Hibernate-E medium (Life technology). The hemispheres were then incubated in 10 mL of Hibernate-E containing 15 U/mL papain (Worthington) for 25 min at 30° C. with gentle mixing every 10 min. Genomic DNA was then digested by prolonging the incubation during 10 min at 37° C. in presence of 4 U/mL rDNase I (Ambion). The obtained suspension was then centrifuged at 800 g for 5 min and the cell pellet was resuspended in 2 mL Hibernate-E and gently dissociated by pipetting up and down 10 times with a plastic Pasteur pipette resulting in a homogenous cell suspension. This suspension was immediately filtered through a 70 pm cell strainer (MACS® SmartStrainer, Miltenyi), collected in 10 mL Hibernate-E and centrifuged at 800 g for 5 min. The cell pellet was resuspended in Neurobasal medium, supplemented with 2% B-27, 0.5 mM Glutamax-I, 100 U/mL penicillin, 100 pg/mL streptomycin (Life Technologies) and diluted at the final concentration of 300′000 cells/mL. One day before plating the cells, 384-well plates were coated with 25 μL/well of 0.1% poly-L-lysine solution (Sigma), incubated overnight at 37° C., washed two times with sterile distilled water and allowed to dry at room temperature for >2 h. The neurons were seeded at a density of 15′000 cells/well in 50 μL/well in a 384-well black, clear-bottomed plates (Corning) and subsequently maintained in an incubator at 37° C., 5% CO₂ and 95% humidity for 8 to 10 days. After 3 and 7 days, 40% of media was renewed under sterile conditions.

Protocol Rat Oscillation Assay

Neurons seeded in the assay plates were washed with Hank's balanced salt solution (HBSS) devoid of Ca²⁺ and Mg²⁺, supplemented with 20 mM HEPES (Life Technologies) and 2 mM CaCl₂ (Sigma), pH 7.4 (hereafter called Assay buffer) using a Biotek EL406 plate washer. Neurons were loaded with 1 μM Fluo-8 AM in Assay buffer for 15 min at 37° C., 5% CO₂. Buffer containing dye was then removed and the assay plates were washed 3 times with Assay buffer using the Biotek EL406 washer and allowed to equilibrate in 50 μL of assay buffer at room temperature for 25 min. The kinetic curves of fluorescence fluctuations acquired once per second using FLIPR® Tetra reflect neuronal calcium oscillations. Recording was performed in two phases separated by 20 min resulting in two acquisitions: “Acute” and “20 min”. In the “Acute” acquisition phase, fluorescence was recorded over a period of 500 sec in presence or absence of the Kᵥ7 channel blocker XE-991. Test compounds were added 250 sec after acquisition start. 20 min after compound addition, calcium oscillations were recorded again for 400 sec, corresponding to the “20 min” acquisition phase.

Stock solutions of test compounds were prepared at a concentration of 10 mM in DMSO (Sigma). 5-fold serial dilutions of the compounds were first prepared in DMSO. Compounds were then diluted in Assay buffer supplemented with 0.1% fatty-acid free bovine serum albumin (Sigma), reaching final compound concentrations of 128 μM to 10 μM on the neurons. The Kᵥ7 channel blocker XE-991 (Biotrend) was directly diluted in Assay buffer containing 0.1% fatty-acid free bovine serum albumin, yielding a final concentration of 10 μM in the assay plate.

Analysis

Time-sequence data were exported using Screenworks® software (Molecular Devices) and converted with Orbit software (Idorsia Pharmaceuticals ltd.) to a format compatible with proprietary analysis softwares. A high-pass filter was then applied to flatten the signal using HTStudio (Idorsia Pharmaceuticals ltd.) to allow calculations of areas under the curve (AUC) for all time-point and compound concentrations. This allowed to calculate potencies (1050) at both “Acute” phase and “20 min” phase (“IC₅₀acute” and “IC₅₀20 min”) using IC50Studio (Idorsia Pharmaceuticals ltd.) as described hereafter. Note: alternatively, signal flattening and 1050 calculations can be achieved using commercially available softwares such as Igor Pro® from Wave Metric (“moving window” filter) and Prism 7.0 from GraphPad, respectively.
“IC₅₀acute”: the ratio of AUC fluorescence before and after compound addition was used to generate concentration-response curve (inhibition) using non-linear regression analysis with a 4-parameter fitting
“IC₅₀20 min”: the AUC of fluorescence measured 20 min after compounds addition was used to generate concentration-response curve (inhibition) using non-linear regression analysis with a 4-parameter fitting.

IC₅₀ value corresponds to the compound concentration that inhibits 50% of the neuronal oscillations in the presence of vehicle (top plateau). The maximum of inhibition corresponds to the full abolishment of oscillations (bottom plateau), which was obtained by addition of 100 μM carbamazepine (Sigma).

Shift value was calculated as follows: Shift value=(IC₅₀ acute value in presence of 10 μM XE-991 [nM])/(IC₅₀acute value [nM]). If IC₅₀ in presence of XE-991 could not be calculated, then the minimal Shift value was calculated as follows: Shift value=(highest tested concentration [nM])/(IC₅₀acute value [nM]), and the Shift value was annotated with “>”.

TABLE 1
Rat oscillation IC₅₀s and shift

| | FLIPR: IC₅₀ [nM] | | |
|---|---|---|---|
| Example No | acute | 20 min | Shift |
| 1 | 27 | 20 | 461 |
| 2 | 63 | 86 | 64 |
| 3 | 122 | 186 | 18 |
| 4 | 175 | 152 | 21 |
| 5 | 82 | 156 | 26 |
| 6 | 62 | 112 | 31 |
| 7 | 85 | 142 | 14 |
| 8 | 78 | 35 | 33 |
| 9 | 5.1 | 2.1 | 85 |
| 10 | 6.2 | 6.0 | 1500 |
| 11 | 246 | 171 | 32 |
| 12 | 36 | 55 | 52 |
| 13 | 88 | 133 | 40 |
| 14 | 31 | 27 | 35 |
| 15 | 124 | 126 | 14 |
| 16 | 224 | 196 | 17 |
| 17 | 141 | 145 | 22 |
| 18 | 109 | 65 | 23 |
| 19 | 337 | 274 | 2.7 |
| 20 | 508 | 304 | 5.1 |
| 21 | 112 | 72 | 12 |
| 22 | 275 | 268 | 8.4 |
| 23 | 13 | 4.6 | 74 |
| 24 | 14 | 8.6 | 94 |
| 25 | 10 | 19 | 214 |
| 26 | 190 | 206 | 10 |
| 27 | 278 | 169 | 5.0 |
| 28 | 629 | 600 | 5.4 |
| 29 | 452 | 253 | 5.8 |
| 30 | 252 | 269 | 18 |
| 31 | 20 | 25 | 155 |
| 32 | 24 | 14 | 38 |
| 33 | 80 | 65 | 33 |
| 34 | 6.7 | 8.5 | 221 |
| 35 | 22 | 24 | 136 |
| 36 | 236 | 330 | 105 |
| 37 | 10 | 8.1 | 394 |
| 38 | 627 | 330 | 7.7 |
| 39 | 487 | 571 | 2 |
| 40 | 351 | 382 | 2.7 |
| 41 | 44 | 25 | 95 |
| 42 | 27 | 22 | 70 |
| 43 | 16 | 22 | 74 |
| 44 | 78 | 100 | 10 |
| 45 | 549 | 628 | 8.8 |
| 46 | 234 | 285 | 12 |
| 47 | 103 | 222 | 9.3 |
| 48 | 510 | 138 | 2 |
| 49 | 680 | 1530 | 4.5 |
| 50 | 80 | 118 | 16 |
| 51 | 80 | 65 | 249 |
| 52 | 72 | 143 | 19 |
| 53 | 185 | 226 | 4.4 |
| 54 | 207 | 244 | 2.3 |
| 55 | 67 | 48 | 7.6 |
| 56 | 27 | 9.7 | 13 |
| 57 | 2.9 | 2.3 | 6.9 |
| 58 | 20 | 16 | 39 |
| 59 | 45 | 61 | 81 |
| 60 | 109 | 144 | 3.4 |
| 61 | 14 | 25 | 868 |
| 62 | 50 | 65 | 9.6 |
| 63 | 46 | 92 | 86 |
| 64 | 44 | 47 | 28 |
| 65 | 6.7 | 10 | 45 |
| 66 | 8.2 | 17 | 231 |
| 67 | 10 | 11 | 65 |
| 68 | 18 | 18 | 14 |
| 69 | 95 | 83 | 48 |
| 70 | 219 | 546 | 17 |
| 71 | 92 | 105 | 6.2 |
| 72 | 266 | 238 | 2.5 |
| 73 | 305 | 244 | 4.5 |
| 74 | 27 | 30 | 82 |
| 75 | 106 | 129 | 34 |
| 76 | 19 | 17 | 312 |
| 77 | 279 | 226 | 3.5 |
| 78 | 9.4 | 7.2 | 101 |
| 79 | 599 | 634 | 2.2 |
| 80 | 13 | 14 | 64 |
| 81 | 2175 | 941 | 2.4 |
| 82 | 66 | 59 | 30 |
| 83 | 93 | 100 | 186 |
| 84 | 5.1 | 4.2 | 733 |
| 85 | 157 | 146 | 45 |
| 86 | 110 | 91 | 47 |
| 87 | 21 | 12 | 111 |
| 88 | 45 | 28 | 88 |
| 89 | 75 | 74 | 30 |
| 90 | 4.0 | 2.7 | 83 |
| 91 | 3.4 | 1.4 | 404 |
| 92 | 1.3 | 0.8 | 527 |
| 93 | 1.9 | 2.2 | 236 |
| 94 | 197 | 117 | 6.6 |
| 95 | 52 | 48 | 31 |
| 96 | 82 | 61 | 24 |
| 97 | 53 | 170 | 103 |
| 98 | 11 | 14 | 108 |
| 99 | 32 | 20 | 3.3 |
| 100 | 5.9 | 3.6 | 1099 |
| 101 | 2.4 | 1.6 | 201 |
| 102 | 2.8 | 0.5 | 446 |
| 103 | 4.8 | 7.4 | 2021 |
| 104 | 292 | 295 | 6.3 |
| 105 | 278 | 261 | 11 |
| 106 | 535 | 757 | 5.6 |
| 107 | 72 | 91 | 27 |
| 108 | 205 | 279 | 4.6 |
| 109 | 200 | 262 | 3.9 |
| 110 | 88 | 62 | 32 |
| 111 | 26 | 34 | 4.2 |
| 112 | 1104 | 1425 | 3.1 |
| 113 | 33 | 22 | 24 |
| 114 | 107 | 61 | 5.9 |
| 115 | 185 | 241 | 64 |
| 116 | 530 | 609 | 2.3 |
| 117 | 195 | 286 | 23 |
| 118 | 116 | 81 | 60 |
| 119 | 10 | 12 | 192 |
| 120 | 136 | 151 | 72 |
| 121 | 990 | 1210 | 7.8 |
| 122 | 339 | 658 | 12 |
| 123 | 395 | 367 | 29 |
| 124 | 69 | 280 | 49 |
| 125 | 1820 | 1990 | 2.2 |
| 126 | 4960 | 7620 | 3.2 |
| 127 | 19 | 22 | 95 |
| 128 | 981 | 950 | 3.7 |
| 129 | 212 | 254 | 6.3 |
| 130 | 30 | 42 | 44 |
| 131 | 29 | 52 | 37 |
| 132 | 79 | 110 | 21 |
| 133 | 396 | 670 | 14 |
| 134 | 63 | 82 | 7.3 |
| 135 | 133 | 115 | 8.8 |
| 136 | 334 | 386 | 13 |
| 137 | 1240 | 1015 | 2.4 |
| 138 | 60 | 92 | 43 |
| 139 | 186 | 365 | 352 |
| 140 | 27 | 59 | 96 |
| 141 | 22 | 26 | 23 |
| 142 | 204 | 277 | 8.3 |
| 143 | 42 | 61 | 69 |
| 144 | 96 | 119 | 101 |
| 145 | 57 | 80 | 9.6 |
| 146 | 4.2 | 6.6 | 72 |
| 147 | 18 | 23 | 109 |
| 148 | 312 | 355 | 4.1 |
| 149 | 98 | 103 | 21 |
| 150 | 1077 | 1160 | 10 |
| 151 | 269 | 419 | 6.3 |
| 152 | 520 | 532 | 50 |
| 153 | 336 | 601 | 16 |
| 154 | 8.1 | 24 | 18 |
| 155 | 151 | 163 | 83 |
| 156 | 60 | 83 | 67 |
| 157 | 74 | 156 | 15 |
| 158 | 390 | 414 | 15 |
| 159 | 26 | 47 | 26 |
| 160 | 83 | 253 | 220 |
| 161 | 78 | 97 | 12 |
| 162 | 120 | 124 | 8.0 |
| 163 | 204 | 282 | 3.3 |
| 164 | 267 | 619 | 3.8 |
| 165 | 145 | 289 | 13 |
| 166 | 889 | 474 | 2.7 |
| 167 | 1275 | 1750 | 8.7 |
| 168 | 3.7 | 3.0 | 1537 |
| 169 | 42 | 20 | 17 |
| 170 | 76 | 59 | 26 |
| 171 | 252 | 377 | 7.7 |
| 172 | 38 | 37 | 107 |
| 173 | 7.0 | 11 | 513 |
| 174 | 2870 | 2490 | 3.7 |
| 175 | 9.0 | 5.5 | 797 |
| 176 | 2.9 | 4.3 | 143 |
| 177 | 22 | 24 | 43 |
| 178 | 16 | 11 | 137 |
| 179 | 24 | 13 | 181 |
| 180 | 62 | 78 | 150 |
| 181 | 404 | 564 | 15 |
| 182 | 11 | 13 | 75 |
| 183 | 1415 | 1990 | 2.5 |
| 184 | 8.8 | 5.7 | 351 |
| 185 | 936 | 753 | 3.2 |
| 186 | 20 | 40 | 231 |
| 187 | 2.7 | 3.9 | 2616 |
| 188 | 5.3 | 5.6 | 769 |
| 189 | 221 | 196 | 99 |
| 190 | 286 | 200 | 2.7 |

8) Kᵥ7.2/3 assay:

HEK293 cells were stably transfected with the appropriate ion channel cDNA(s) (human KCNQ2 and KCNQ3 genes). Cells were cultured in Dulbecco's Modified Eagle Medium/Nutrient mixture F-12 (D-MEM/F-12) supplemented with 10% fetal bovine serum, 100 U/mL penicillin G sodium, 100 pg/mL streptomycin sulfate and selection antibiotics. FLIPR Test Procedure: For FLIPR assay, cells were plated in 384-well black clear-bottomed microtiter plates (BD Biocoat Poly-D-Lysine Multiwell Cell Culture Plate) at 15'000 to 30'000 cells per well. Cells were incubated at 37° C. overnight or until cells reached sufficient density in the wells (near confluent monolayer) to use in fluorescence assays. Fluorescence changes triggered by agonist application were recorded using FLIPR® Tetra and displayed with Screenworks® 4.2 software (Molecular Devices). Assays were performed with the FLIPR potassium assay kit (Molecular Devices) according to the manufacturer's instructions. Dye-loading: Growth media was removed and replaced with 20 μL of dye loading buffer for 60 min at room temperature. FLIPR Recording (agonist mode): Stock solutions of test compounds were prepared at a concentration of 33.3 mM in DMSO. 5 μL of 5× concentrated test, vehicle, or control compounds solutions prepared in the stimulation buffer (K⁺-free buffer with 5 mM TI⁺) were added to each well and fluorescence recording was continued for 5 min. The agonist effect (EC₅₀ and % effect) of test or control compounds on Kᵥ7 channels was determined as follows: Raw data was exported using Screenworks® 4.2 software and the fluorescence traces were analysed using Microsoft Excel (Microsoft Corp., Redmond, Wash.). The test compounds responses were expressed as % of maximum response of the control compound Flupirtine (Sigma-Aldrich), which was tested at concentrations ranging from 0.03 to 100 μM. Concentration-response data were fitted to a Hill equation. Non-linear least squares fits were made assuming a simple binding model. If appropriate, fits were weighted by the standard deviation. No assumptions about the fit parameters were made; the fit parameters were determined by the algorithm.

TABLE 2
Kᵥ7.2/7.3 activation

| Example No | FLIPR: EC₅₀ (nM) |
|---|---|
| 1 | 30 |
| 2 | 70 |
| 37 | 1.5 |
| 59 | 12.4 |
| 60 | 42 |
| 103 | 1.5 |
| 116 | 161 |
| 175 | 30 |
| 177 | 7.0 |
| 186 | 23 |

III. Pharmacological Experiments

Formulation and Administration.

Compounds were formulated in a 10% polyethylene glycol 400 (PEG 400)/90% aqueous solution of 0.5% methylcellulose (MC 0.5%). Firstly drugs are dissolved in PEG 400 and then suspended in MC 0.5% for oral gavage at X mg/5 mL/kg (X see table).

Audiogenic Seizure-Sensitive Mouse Model of Generalized Convulsive Seizures

1. Procedure: Following two days of acclimatisation, auditory seizures are induced in male juvenile DBA/2J mice (22-24 days old). Each mouse is placed individually in the exposure chamber, an hemispheric acrylic glass dome (diameter: 50 cm) within a sound-attenuated box. The sound-attenuated box is equipped with two house lights and a camera system (Fire-I from Unibrain) in order to observe and record the behavioral seizure response. After 60 seconds of habituation, the stimulus, a mixed frequency tone of 15-20 kHz at 110 dB (SASLab Lite, Avisoft Bioacoustics), is played from a speaker that is placed on the top center of the dome. The stimulus is applied for 60 seconds maximum or until the mouse shows tonic extension of the hind limbs. Seizures are classified as following: stage 0, normal behavior; stage 1, wild running; stage 2, generalized clonus; stage 3, tonic extension of the hind limbs.

2. Compounds testing: Acute compound effects on audiogenic generalized convulsive seizures are evaluated in independent groups of 8-10 mice randomly assigned. Following oral administration of compound or vehicle, the maximum seizure stage during sound exposure is assessed. Compounds are given 1 hour before exposure to the stimulus. Each mouse is exposed to the auditory stimulus only once and euthanized afterwards by CO₂ inhalation.

TABLE 3
Efficacies in the AGS mouse model

| | | Dose and | Time of | Seizure stage |
|---|---|---|---|---|
| Example No | Formulation | administration | challenge | [% vs. vehicle] |
| 2 | 90% MC 0.5% + | 30 mg/kg | 1 h | −95 |
| | 10% PEG400 | po | | |
| 59 | 90% MC 0.5% + | 10 mg/kg | 1 h | −70 |
| | 10% PEG400 | po | | |
| 100 | 90% MC 0.5% + | 33 mg/kg | 1 h | −71 |
| (HCl salt) | 10% PEG400 | po | | |
| 155 | 90% MC 0.5% + | 30 mg/kg | 1 h | −100 |
| | 10% PEG400 | po | | |
| 159 | 90% MC 0.5% + | 30 mg/kg | 1 h | −100 |
| | 10% PEG400 | po | | |
| 160 | 90% MC 0.5% + | 30 mg/kg | 1 h | −100 |
| | 10% PEG400 | po | | |
| 172 | 90% MC 0.5% + | 30 mg/kg | 1 h | −81 |
| | 10% PEG400 | po | | |
| 182 | 90% MC 0.5% + | 5 mg/kg | 1 h | −100 |
| | 10% PEG400 | po | | |
| 186 | 90% MC 0.5% + | 5 mg/kg | 1 h | −67 |
| | 10% PEG400 | | | |

Amygdala-Kindling Rat Model:

1. Procedure: Adult male Wistar rats (Harlan Laboratories, Netherlands, or Charles Rivers, Germany; body weight 300-350 g) were stereotaxically implanted with twisted bipolar plastic-coated stainless steel electrode (MS333-2-BIU 10 mm, Plastics One) into the right basolateral amygdala under isoflurane anesthesia. To place the electrode, trepanations were made in the skull and the electrode was lowered into the right basolateral amygdala (from bregma: anteriorposterior (AP): −2.5 mm, medio-lateral (ML): −3.5 mm, dorso-ventral (DV): −8.6 mm; α=10°) and secured to the skull with screws and dental acrylate. After one week of recovery, they were handled daily and habituated over one week to the kindling set-up. Kindling procedure: For a kindling session each rat was placed individually into a smooth acrylic plastic, round-bottomed bowl (Ø 36 cm, height 36 cm, BASi movement-responsive caging system) and its intracranial implanted electrode was connected to the stimulator (STG4008, Multichannel Systems GmbH) and the recording devices (PowerLab 8/35, ADInstruments Ltd) via a cable (335-340/3 (C), Plastics One). For the kindling procedure, each rat was exposed once daily to an electrical stimulation and the behavioral symptoms of the evoked seizure were observed and classified according to the modified Racine scale (stage 0, arrest, wet dog shakes, normal behaviour; stage 1, facial twitches: nose, lips, eyes; stage 2, chewing, head nodding; stage 3, forelimb clonus; stage 4, rearing, falling on forelimbs; stage 5, rearing, falling on side or back, rolling). The electrical stimulus consists of a 1s-train of 50 Hz square-wave biphasic pulses of 1-ms duration at an intensity of 400 μA (suprathreshold intensity). The stimulus was applied daily until each rat was fully kindled, i.e. it showed seizures of severity stage 4 and 5 upon electrical stimulation in at least ten consecutive kindling sessions. Data Scoring and analysis. The duration of electroencephalographic seizures (afterdischarge, AD) was recorded using LabChart7 Pro software (ADlnstruments Ltd). Simultaneously, videos were recorded to evaluate seizure stage (SS).

2. Compound testing: Acute drug effects were evaluated in groups of 6-8 fully kindled rats in a randomized cross-over design with 48 h between drug and vehicle applications. Following oral administration of drug or vehicle, drug testing included determination of the afterdischarge threshold (the minimal stimulation intensity necessary to evoke an afterdischarge (electroencephalographically measured neuronal hyper-synchronous activity with an amplitude 2-times higher than baseline amplitude and a frequency of ≥1 Hz) of at least 3 sec duration) and monitoring electroencephalographic and behavioral correlates of the evoked seizure at ADT (afterdischarge threshold), including AD duration and SS, by a experimenter blind to treatment assignment.

TABLE 4
Efficacies in the rat kindling model

| | | Dose and | Time of | Seizure stage | AD duration |
|---|---|---|---|---|---|
| Example No | Formulation | administration | challenge | [% vs veh] | [% vs veh] |
| 2 | 90% MC 0.5% + | 100 mg/kg | 1 h | −88 | −93 |
| | 10% PEG400 | po | | | |
| 59 | 90% MC 0.5% + | 30 mg/kg | 1 h | −83 | −96 |
| | 10% PEG400 | po | | | |
| 168 | 90% MC 0.5% + | 1 mg/kg | 30 min | −58 | −84 |
| (HCl salt) | 10% PEG400 | po | | | |
| 186 | 90% MC 0.5% + | 10 mg/kg | 15 min | −50 | −87 |
| | 10% PEG400 | po | | | |

1. A compound of Formula (I) 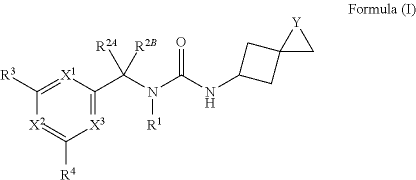
wherein
X¹ represents nitrogen or CR^{X1}; wherein R^{X1} represents hydrogen, halogen, (C₁₋₄)alkyl, or (C₁₋₄)alkoxy;
X² represents nitrogen or CR^{X2}; wherein R^{X2} represents hydrogen, halogen, (C₁₋₄)alkyl, or (C₁₋₄)alkoxy;
X³ represents nitrogen or CR^{X3}; wherein R^{X3} represents hydrogen, halogen, (C₁₋₄)alkyl, (C₁₋₄)alkoxy, or hydroxy;
R¹ represents hydrogen, or methyl; or
R¹ and R^{X1} together represent a —CH₂CH₂— bridge;
Y represents —C(R^{Y1})(R^{Y2})—, or *—CH₂—C(R^{Y3})(R^{Y4})—, wherein the asterisk indicates the bond which is linked to the cyclobutyl ring;
R^{Y1} represents hydrogen, or fluoro;
R^{Y2} represents hydrogen or fluoro;
R^{Y3} represents hydrogen, fluoro or iodo;
R^{Y4} represents hydrogen or fluoro;
R^{2A} represents hydrogen; (C₁₋₄)alkyl; (C₂₋₄)alkenyl; (C₂₋₄)alkynyl; (C₃₋₆)cycloalkyl; (C₁₋₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋₂)alkyl; (C₁₋₂)alkyl-S—(C₁₋₂)alkyl; (C₁₋₂)alkyl-(SO₂)—(C₁₋₂)alkyl; cyano; (C₁₋₂)cyanoalkyl; H₂N—C(O)—(C₁₋₂)alkyl; (R^{N1})₂N—(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl;
and R^{2B} represents hydrogen, or methyl; or
R^{2A} and R^{2B} form, together with the carbon atom to which they are attached, a ring of 3- to 6 members, wherein the members needed to complete said ring are each independently selected from —CH₂— and O— and wherein said ring does not contain more than one —O— member; or
R^{2A} and R^{X3} together represent a —CH₂—O—* bridge, wherein the asterisk indicates the bond which is linked to the aromatic carbon atom, and
R^{2B} represents hydrogen;
R³ represents
SF₅;
(C₁₋₂)alkyl-S(O)₂—; or
a fragment 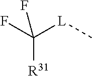
wherein
R³¹ represents hydrogen, or fluoro; and
L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋₄)alkyl, CH₃—O—CH₂—, or (CH₃)₂NCH₂—;
wherein the asterisks indicate the bond which is linked to the aromatic carbon atom; or
R³ and R^{X1} together represent a —O—CF₂—O— bridge; or
R³ and R^{X2} together represent a —O—CF₂—O— bridge; and
R⁴ represents hydrogen, halogen, or (C₁₋₄)alkyl;
or a salt thereof.

2. A compound according to claim 1, wherein R¹ represents hydrogen; or a salt thereof.

3. A compound according to claim 2, wherein R^{Y1}R^{Y2}, R^{Y3} and R^{Y4} all represent hydrogen; or a salt thereof.

4. A compound according to claim 2, wherein
R^{2A} represents hydrogen, (C₁₋₄)alkyl, (C₃₋₆)cycloalkyl, (C₁₋₄)fluoroalkyl, (C₁₋₄)hydroxyalkyl, (C₁₋₄)alkoxy-(C₁₋₂)alkyl, (C₁₋₂)alkyl-(SO₂)—(C₁₋₂)alkyl, cyano, (C₁₋₂)cyanoalkyl, H₂N—C(O)—(C₁₋₂)alkyl, (R^{N1})₂N—(C₁₋₂)alkyl, or (R^{N1})₂N—C(O)—, wherein R^{N1} independently represents hydrogen or (C₁₋₂)alkyl;
and R^{2B} represents hydrogen; or
R^{2A} and R^{2B} form, together with the carbon atom to which they are attached, a ring of 3- to 6 members selected from cycloprop-1,1-diyl, cyclobut-1,1-diyl, oxetane-3,3-diyl and tetrahydropyran-4,4-diyl;
or a salt thereof.

5. A compound according to claim 2, wherein R^{2A} represents hydrogen, methyl, or hydroxymethyl; and R^{2B} represents hydrogen;
or a salt thereof.

6. A compound according to claim 2, wherein R³ represents a fragment 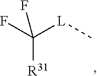
wherein
R³¹ represents hydrogen, or fluoro; and
L represents a direct bond, —CHR^{L}—O*, —O—CH₂—*, —CH₂—NH—*, —CH₂—N(CH₃)—*, or —O—; wherein R^{L} represents hydrogen, methyl, CH₃—O—CH₂—, or (CH₃)₂NCH₂—; wherein the asterisks indicate the bond which is linked to the aromatic carbon atom;
or a salt thereof.

7. A compound according to claim 2, wherein R⁴ represents hydrogen;
or a salt thereof.

8. A compound according to claim 2, wherein the fragment 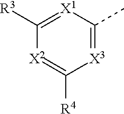
represents: 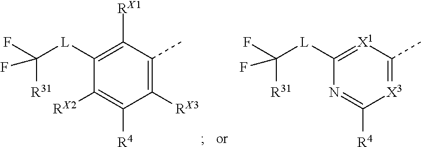
wherein
X³ represents nitrogen and X¹ represents CR^{X1};
X¹ represents nitrogen and X³ represents CR^{X3}; or
X¹ represents CR^{X1} and X³ represents CR^{X3};
or a salt thereof.

9. A compound according to claim 2, wherein the fragment 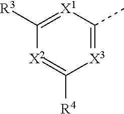
represents: 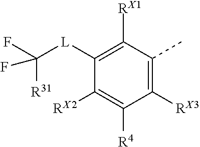
wherein R^{X1} represents hydrogen, fluoro, chloro, methyl, or methoxy; R^{X2} represents hydrogen; R^{X3} represents hydrogen, fluoro, or chloro; R⁴ represents hydrogen, fluoro, chloro, or methyl; R³¹ represents hydrogen, or fluoro; and L represents a direct bond, —CHR^{L}—O—*, —CH₂—NH—*, —CH₂—N(CH₃)—*, —O—, or; wherein R^{L} represents hydrogen, or methyl; wherein the asterisks indicate the bond which is linked to the aromatic carbon atom; or 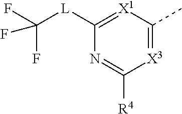
wherein
X³ represents nitrogen and X¹ represents CR^{X1};
X¹ represents nitrogen and X³ represents CR^{X3}; or
X¹ represents CR^{X1} and X³ represents CR^{X3};
wherein R^{X1} represents hydrogen, or methoxy; R^{X3} represents hydrogen; R⁴ represents hydrogen, or methyl; and L represents —CHR^{L}—O*, —CH₂—NH—*, or —CH₂—N(CH₃)—*; wherein R^{L} represents hydrogen, methyl, CH₃—O—CH₂—, or (CH₃)₂NCH₂—; wherein the asterisks indicate the bond which is linked to the aromatic carbon atom;
or a salt thereof.

10. A compound according to claim 1, wherein
X¹ represents CR^{X1}; wherein R^{X1} represents hydrogen;
X² represents nitrogen or CR^{X2}; wherein R^{X2} represents hydrogen;
X³ represents nitrogen or CR^{X3}; wherein R^{X3} represents hydrogen;
R¹ represents hydrogen;
Y represents —CH₂—, or —CH₂—CH₂—;
R^{2A} represents hydrogen, or hydroxymethyl;
R^{2B} represents hydrogen;
R³ represents trifluoromethyl or 2,2,2-trifluoroethoxy; and
R⁴ represents hydrogen;
or a salt thereof.

11. A compound according to claim 1 wherein said compound is:
1-Spiro[3.3]hept-2-yl-3-(3-trifluoromethyl-benzyl)-urea;
1-Spiro[2.3]hex-5-yl-3-(3-trifluoromethyl-benzyl)-urea;
1-(1,1-Difluoro-spiro[2.3]hex-5-yl)-3-(3-trifluoromethyl-benzyl)-urea;
1-((3r,5r)-1,1-Difluoro-spiro[2.3]hex-5-yl)-3-(3-trifluoromethyl-benzyl)-urea;
1-((3s,5s)-1,1-Difluoro-spiro[2.3]hex-5-yl)-3-(3-trifluoromethyl-benzyl)-urea;
1-(6,6-Difluoro-spiro[3.3]hept-2-yl)-3-(3-trifluoromethyl-benzyl)-urea;
1-[1-(3-Difluoromethoxy-phenyl)-cyclopropyl]-3-spiro[3.3] hept-2-yl-urea;
1-(2-Fluoro-3-trifluoromethoxy-benzyl)-3-spiro[3.3]hept-2-yl-urea;
1-[2-Methoxy-1-(3-trifluoromethyl-phenyl)-ethyl]-3-spiro[3.3] hept-2-yl-urea;
1-[1-(2-Difluoromethoxy-pyridin-4-yl)-2-methoxy-ethyl]-3-spiro[3.3]hept-2-yl-urea;
1-Spiro[3.3] hept-2-yl-3-(5-trifluoromethoxy-2,3-dihydro-benzofuran-3-yl)-urea;
1-(2-Fluoro-3-trifluoromethyl-benzyl)-3-spiro[3.3]hept-2-yl-urea;
1-(3-Difluoromethoxy-benzyl)-3-spiro[3.3]hept-2-yl-urea;
1-Spiro[3.3] hept-2-yl-3-[3-(2,2,2-trifluoro-ethoxy)-benzyl]-urea;
1-(3-Difluoromethyl-benzyl)-3-spiro[3.3]hept-2-yl-urea;
1-(2-Fluoro-5-trifluoromethyl-benzyl)-3-spiro[3.3]hept-2-yl-urea;
1-(4-Fluoro-3-trifluoromethyl-benzyl)-3-spiro[3.3]hept-2-yl-urea;
1-(3-Fluoro-5-trifluoromethyl-benzyl)-3-spiro[3.3]hept-2-yl-urea;
1-[1-(2-Bromo-5-trifluoromethyl-phenyl)-ethyl]-3-spiro[3.3]hept-2-yl-urea;
1-[1-(2-Methoxy-5-trifluoromethyl-phenyl)-ethyl]-3-spiro[3.3] hept-2-yl-urea;
1-[1-(2-Methoxy-3-trifluoromethyl-phenyl)-ethyl]-3-spiro[3.3] hept-2-yl-urea;
1-(2-Methoxy-3-trifluoromethyl-benzyl)-3-spiro[3.3] hept-2-yl-urea;
1-Spiro[3.3]hept-2-yl-3-[1-(3-trifluoromethyl-phenyl)-ethyl]-urea;
1-[Cyclopropyl-(3-trifluoromethyl-phenyl)-methyl]-3-spiro[3.3]hept-2-yl-urea;
1-[Cyclopropyl-(3-difluoromethoxy-phenyl)-methyl]-3-spiro[3.3]hept-2-yl-urea;
1-{1-[4-Methyl-3-(2,2,2-trifluoro-ethoxy)-phenyl]-ethyl}-3-spiro[3.3]hept-2-yl-urea;
1-{1-[4-Chloro-3-(2,2,2-trifluoro-ethoxy)-phenyl]-ethyl}-3-spiro[3.3]hept-2-yl-urea;
1-{1-[4-Methoxy-3-(2,2,2-trifluoro-ethoxy)-phenyl]-ethyl}-3-spiro[3.3]hept-2-yl-urea;
1-{1-[2-Methyl-3-(2,2,2-trifluoro-ethoxy)-phenyl]-ethyl}-3-spiro[3.3]hept-2-yl-urea;
1-{1-[2-Chloro-3-(2,2,2-trifluoro-ethoxy)-phenyl]-ethyl}-3-spiro[3.3]hept-2-yl-urea;
1-{1-[2-Methoxy-3-(2,2,2-trifluoro-ethoxy)-phenyl]-ethyl}-3-spiro[3.3]hept-2-yl-urea;
1-{1-[3-Methyl-5-(2,2,2-trifluoro-ethoxy)-phenyl]-ethyl}-3-spiro[3.3]hept-2-yl-urea;
1-{1-[3-Chloro-5-(2,2,2-trifluoro-ethoxy)-phenyl]-ethyl}-3-spiro[3.3]hept-2-yl-urea;
1-(1-(3-(pentafluoro-16-sulfaneyl)phenyl)ethyl)-3-(spiro[3.3]heptan-2-yl)urea;
1-(3-(pentafluoro-16-sulfaneyl)benzyl)-3-(spiro[3.3]heptan-2-yl)urea;
1-[1-(3-Methanesulfonyl-phenyl)-ethyl]-3-spiro[3.3] hept-2-yl-urea;
1-Spiro[3.3]hept-2-yl-3-[3-(1-trifluoromethyl-cyclopropyl)-benzyl]-urea;
1-(2-Hydroxy-5-trifluoromethyl-benzyl)-3-spiro[3.3]hept-2-yl-urea;
1-{1-[2-Chloro-5-(2,2,2-trifluoro-ethoxy)-phenyl]-ethyl}-3-spiro[3.3]hept-2-yl-urea;
1-{1-[2-Methoxy-5-(2,2,2-trifluoro-ethoxy)-phenyl]-ethyl}-3-spiro[3.3]hept-2-yl-urea;
1-Spiro[3.3]hept-2-yl-3-(3-trifluoromethoxy-benzyl)-urea;
1-Spiro[3.3]hept-2-yl-3-(3-trifluoromethanesulfonyl-benzyl)-urea;
1-Spiro[3.3]hept-2-yl-3-(3-trifluoromethoxymethyl-benzyl)-urea;
1-Spiro[3.3]hept-2-yl-3-[3-(3-trifluoromethyl-phenyl)-oxetan-3-yl]-urea;
1-[2-Hydroxy-2-methyl-1-(3-trifluoromethyl-phenyl)-propyl]-3-spiro[3.3] hept-2-yl-urea;
1-(2,2-Difluoro-benzo[1,3]dioxol-4-ylmethyl)-3-spiro[3.3]hept-2-yl-urea;
1-[1-(2,2-Difluoro-benzo[1,3]dioxol-4-yl)-ethyl]-3-spiro[3.3]hept-2-yl-urea;
1-{2-Dimethylamino-1-[6-(2,2,2-trifluoro-ethoxy)-pyridin-2-yl]-ethyl{-3-spiro[3.3]hept-2-yl-urea;
1-Spiro[3.3]hept-2-yl-3-{4-[2-(2,2,2-trifluoro-ethoxy)-pyridin-4-yl]-tetrahydro-pyran-4-yl}-urea;
1-{2-Methoxy-1-[2-(2,2,2-trifluoro-ethoxy)-pyridin-4-yl]-ethyl}-1-methyl-3-spiro[3.3]hept-2-yl-urea;
1-[1-(2-Difluoromethoxy-pyridin-4-yl)-cyclobutyl]-3-spiro[3.3]hept-2-yl-urea;
1-[1-(2-Difluoromethoxy-pyridin-4-yl)-1-methyl-ethyl]-3-spiro[3.3] hept-2-yl-urea;
5-(2,2,2-Trifluoro-ethoxy)-3,4-dihydro-1H-[2,6]naphthyridine-2-carboxylic acid spiro[3.3]hept-2-ylamide;
1-Methoxymethyl-5-(2,2,2-trifluoro-ethoxy)-3,4-dihydro-1H-isoquinoline-2-carboxylic acid spiro[3.3]hept-2-ylamide;
1-[Cyano-(3-trifluoromethyl-phenyl)-methyl]-3-spiro[3.3]hept-2-yl-urea;
1-Spiro[3.3]hept-2-yl-3-[(1H-[1,2,4]triazol-3-yl)-(3-trifluoromethyl-phenyl)-methyl]-urea;
1-[2,2-Difluoro-1-(3-trifluoromethyl-phenyl)-ethyl]-3-spiro[3.3]hept-2-yl-urea;
1-Spiro[3.3]hept-2-yl-3-[2,2,2-trifluoro-1-(3-trifluoromethyl-phenyl)-ethyl]-urea;
1-Spiro[3.3]hept-2-yl-3-[6-(2,2,2-trifluoro-ethoxy)-pyrimidin-4-ylmethyl]-urea;
1-[2-Methyl-6-(2,2,2-trifluoro-ethoxy)-pyrimidin-4-ylmethyl]-3-spiro[3.3] hept-2-yl-urea;
1-Spiro[3.3]hept-2-yl-3-{1-[6-(2,2,2-trifluoro-ethoxy)-pyrimidin-4-yl]-ethyl}-urea;
2-(3-Spiro[3.3]hept-2-yl-ureido)-2-(3-trifluoromethyl-phenyl)-acetamide;
N-Methyl-2-(3-spiro[3.3] hept-2-yl-ureido)-2-(3-trifluoromethyl-phenyl)-acetamide;
1-[2-Methanesulfonyl-1-(3-trifluoromethoxy-phenyl)-ethyl]-3-spiro[3.3]hept-2-yl-urea;
1-[1-(2,2-Difluoro-benzo[1,3]dioxol-5-yl)-ethyl]-3-spiro[3.3]hept-2-yl-urea;
1-[2-Cyano-1-(3-trifluoromethoxy-phenyl)-ethyl]-3-spiro[3.3]hept-2-yl-urea;
1-[2-Cyano-1-(3-trifluoromethyl-phenyl)-ethyl]-3-spiro[3.3]hept-2-yl-urea;
1-{1-[2-Methyl-6-(2,2,2-trifluoro-ethoxy)-pyrimidin-4-yl]-ethyl}-3-spiro[3.3]hept-2-yl-urea;
1-[2-Methanesulfonyl-1-(3-trifluoromethyl-phenyl)-ethyl]-3-spiro[3.3]hept-2-yl-urea;
1-Spiro[3.3]hept-2-yl-3-[2-(2,2,2-trifluoro-ethoxy)-pyrimidin-4-ylmethyl]-urea;
1-[2-(2-Methoxy-ethoxy)-1-(3-trifluoromethyl-phenyl)-ethyl]-3-spiro[3.3] hept-2-yl-urea;
1-[2-Isopropoxy-1-(3-trifluoromethyl-phenyl)-ethyl]-3-spiro[3.3]hept-2-yl-urea;
1-[3-Ethoxy-1-(3-trifluoromethyl-phenyl)-propyl]-3-spiro[3.3]hept-2-yl-urea;
1-[3-Hydroxy-1-(3-trifluoromethyl-phenyl)-propyl]-3-spiro[3.3]hept-2-yl-urea;
1-[3-Methylsulfanyl-1-(3-trifluoromethyl-phenyl)-propyl]-3-spiro[3.3]hept-2-yl-urea;
1-[2-Hydroxy-1-(3-trifluoromethyl-phenyl)-ethyl]-3-spiro[3.3]hept-2-yl-urea;
1-{3-Ethoxy-1-[2-(2,2,2-trifluoro-ethoxy)-pyridin-4-yl]-propyl}-3-spiro[3.3] hept-2-yl-urea;
1-{2-Methoxy-1-[6-(2,2,2-trifluoro-ethoxy)-pyrimidin-4-yl]-ethyl}-3-spiro[3.3]hept-2-yl-urea;
4-(3-Spiro[3.3]hept-2-yl-ureido)-4-(3-trifluoromethyl-phenyl)-butyramide;
1-Spiro[3.3]hept-2-yl-3-[1-(3-trifluoromethyl-phenyl)-but-3-enyl]-urea;
1-[3-Methanesulfonyl-1-(3-trifluoromethyl-phenyl)-propyl]-3-spiro[3.3]hept-2-yl-urea;
1-{2-Hydroxy-1-[2-methyl-6-(2,2,2-trifluoro-ethoxy)-pyrimidin-4-yl]-ethyl}-3-spiro[3.3] hept-2-yl-urea;
1-{2-Hydroxy-1-[6-(2,2,2-trifluoro-ethoxy)-pyrimidin-4-yl]-ethyl}-3-spiro[3.3] hept-2-yl-urea;
1-[3,3-Difluoro-1-(3-trifluoromethyl-phenyl)-propyl]-3-spiro[3.3]hept-2-yl-urea;
1-{3-Hydroxy-1-[6-(2,2,2-trifluoro-ethoxy)-pyrimidin-4-yl]-propyl}-3-spiro[3.3] hept-2-yl-urea;
1-[1-(3-Methanesulfonyl-phenyl)-2-methoxy-ethyl]-3-spiro[3.3] hept-2-yl-urea;
1-{2-Hydroxy-1-[3-(2,2,2-trifluoro-ethoxy)-phenyl]-ethyl}-3-spiro[3.3] hept-2-yl-urea;
1-{3-Hydroxy-1-[3-(2,2,2-trifluoro-ethoxy)-phenyl]-propyl}-3-spiro[3.3]hept-2-yl-urea;
1-[Cyano-(3-trifluoromethyl-phenyl)-methyl]-3-spiro[3.3]hept-2-yl-urea;
1-Spiro[3.3]hept-2-yl-3-{1-[3-(2,2,2-trifluoro-ethoxy)-phenyl]-ethyl}-urea;
1-{2-Methoxy-1-[3-(2,2,2-trifluoro-ethoxy)-phenyl]-ethyl}-3-spiro[3.3]hept-2-yl-urea;
1-{2-Methoxy-1-[2-(2,2,2-trifluoro-ethoxy)-pyridin-4-yl]-ethyl}-3-spiro[3.3]hept-2-yl-urea;
1-Spiro[3.3]hept-2-yl-3-{1-[2-(2,2,2-trifluoro-ethoxy)-pyridin-4-yl]-ethyl}-urea;
1-{2-Dimethylamino-1-[2-(2,2,2-trifluoro-ethoxy)-pyridin-4-yl]-ethyl}-3-spiro[3.3]hept-2-yl-urea;
1-{1-[3-Fluoro-5-(2,2,2-trifluoro-ethoxy)-phenyl]-ethyl}-3-spiro[3.3] hept-2-yl-urea;
1-Spiro[3.3]hept-2-yl-3-(2-trifluoromethoxy-pyridin-4-ylmethyl)-urea;
1-Spiro[3.3]hept-2-yl-3-[1-(2-trifluoromethoxy-pyridin-4-yl)-ethyl]-urea;
1-{1-[2-Fluoro-3-(2,2,2-trifluoro-ethoxy)-phenyl]-ethyl}-3-spiro[3.3] hept-2-yl-urea; 1-{1-[2-Fluoro-5-(2,2,2-trifluoro-ethoxy)-phenyl]-ethyl}-3-spiro[3.3]hept-2-yl-urea;
1-[1-(2-Difluoromethoxy-pyridin-4-yl)-ethyl]-3-spiro[3.3] hept-2-yl-urea;
1-(Spiro[3.3]heptan-2-yl)-3-(1-(2-((1,1,1-trifluoropropan-2-yl)oxy)pyridin-4-yl)ethyl)urea;
1-(Spiro[3.3]heptan-2-yl)-3-(1-(3-((1,1,1-trifluoropropan-2-yl)oxy)phenyl)ethyl) urea;
1-Spiro[3.3]hept-2-yl-3-[2-(2,2,2-trifluoro-ethoxy)-pyridin-4-ylmethyl]-urea;
1-Spiro[3.3]hept-2-yl-3-[6-(2,2,2-trifluoro-1-methoxymethyl-ethoxy)-pyridin-2-ylmethyl]-urea;
1-[6-(1-Dimethylaminomethyl-2,2,2-trifluoro-ethoxy)-pyridin-2-ylmethyl]-3-spiro[3.3] hept-2-yl-urea;
1-[2-(1-Dimethylaminomethyl-2,2,2-trifluoro-ethoxy)-pyridin-4-ylmethyl]-3-spiro[3.3] hept-2-yl-urea;
1-Spiro[3.3]hept-2-yl-3-[2-(2,2,2-trifluoro-1-methoxymethyl-ethoxy)-pyridin-4-ylmethyl]-urea;
1-[5-Methyl-6-(2,2,2-trifluoro-ethoxy)-pyridin-2-ylmethyl]-3-spiro[3.3] hept-2-yl-urea;
1-[4-Methyl-3-(2,2,2-trifluoro-ethoxy)-benzyl]-3-spiro[3.3] hept-2-yl-urea;
1-[3-Fluoro-5-(2,2,2-trifluoro-1-methyl-ethoxy)-benzyl]-3-spiro[3.3]hept-2-yl-urea;
1-Spiro[3.3]hept-2-yl-3-{1-[3-(2,2,2-trifluoro-1-methyl-ethoxy)-phenyl]-cyclopropyl}-urea;
1-Spiro[3.3]hept-2-yl-3-[4-(2,2,2-trifluoro-ethoxy)-pyrimidin-2-ylmethyl]-urea;
1-{1-[4-Fluoro-3-(2,2,2-trifluoro-ethoxy)-phenyl]-ethyl}-3-spiro[3.3] hept-2-yl-urea;
1-[4-Fluoro-3-(2,2,2-trifluoro-ethoxy)-benzyl]-3-spiro[3.3]hept-2-yl-urea;
1-{2-[Methyl-(2,2,2-trifluoro-ethyl)-amino]-pyridin-4-ylmethyl}-3-spiro[3.3]hept-2-yl-urea;
1-Spiro[3.3]hept-2-yl-3-[2-(2,2,2-trifluoro-ethylamino)-pyridin-4-ylmethyl]-urea;
1-Spiro[3.3]hept-2-yl-3-[4-(2,2,2-trifluoro-1-methyl-ethoxy)-pyridin-2-ylmethyl]-urea;
1-Spiro[3.3]hept-2-yl-3-[2-(2,2,2-trifluoro-1-methyl-ethoxy)-pyrimidin-4-ylmethyl]-urea;
1-Spiro[3.3]hept-2-yl-3-[1-(3-trifluoromethoxy-phenyl)-ethyl]-urea;
1-Spiro[2.3]hex-5-yl-3-[6-(2,2,2-trifluoro-1-methyl-ethoxy)-pyrimidin-4-ylmethyl]-urea;
1-[1-(2,2-Difluoro-benzo[1,3]dioxol-4-yl)-ethyl]-3-spiro[2.3]hex-5-yl-urea;
1-{2-Hydroxy-1-[6-(2,2,2-trifluoro-ethoxy)-pyrimidin-4-yl]-ethyl}-3-spiro[2.3] hex-5-yl-urea;
1-{2-Hydroxy-1-[2-methyl-6-(2,2,2-trifluoro-ethoxy)-pyrimidin-4-yl]-ethyl}-3-spiro[2.3] hex-5-yl-urea;
1-[1-(3-Difluoromethoxy-phenyl)-ethyl]-3-spiro[2.3]hex-5-yl-urea;
1-(2-Methoxy-5-trifluoromethyl-benzyl)-3-spiro[2.3] hex-5-yl-urea;
1-Spiro[2.3]hex-5-yl-3-[4-(2,2,2-trifluoro-ethoxy)-pyrimidin-2-ylmethyl]-urea;
1-(1-(3-(pentafluoro-16-sulfaneyl)phenyl)ethyl)-3-(spiro[2.3]hexan-5-yl)urea;
1-(2-Methoxy-3-trifluoromethyl-benzyl)-3-spiro[2.3] hex-5-yl-urea;
1-{1-[2-Fluoro-5-(2,2,2-trifluoro-ethoxy)-phenyl]-ethyl}-3-spiro[2.3] hex-5-yl-urea;
1-(3-(pentafluoro-16-sulfaneyl)benzyl)-3-(spiro[2.3] hexan-5-yl)urea;
1-{1-[2-Fluoro-3-(2,2,2-trifluoro-ethoxy)-phenyl]-ethyl}-3-spiro[2.3] hex-5-yl-urea;
1-{1-[3-Fluoro-5-(2,2,2-trifluoro-ethoxy)-phenyl]-ethyl}-3-spiro[2.3] hex-5-yl-urea;
1-[1-(2-Methoxy-3-trifluoromethyl-phenyl)-ethyl]-3-spiro[2.3] hex-5-yl-urea;
1-(2-Fluoro-3-trifluoromethoxy-benzyl)-3-spiro[2.3]hex-5-yl-urea;
1-Spiro[2.3]hex-5-yl-3-{1-[3-(2,2,2-trifluoro-1-methyl-ethoxy)-phenyl]-cyclopropyl}-urea;
1-[1-(3-Difluoromethoxy-phenyl)-cyclopropyl]-3-spiro[2.3] hex-5-yl-urea;
1-[1-(2-Methoxy-5-trifluoromethyl-phenyl)-ethyl]-3-spiro[2.3] hex-5-yl-urea;
1-[3-Hydroxy-1-(3-trifluoromethoxy-phenyl)-propyl]-3-spiro[2.3]hex-5-yl-urea;
1-[1-(2-Difluoromethoxy-pyridin-4-yl)-ethyl]-3-spiro[2.3] hex-5-yl-urea;
1-Spiro[2.3]hex-5-yl-3-[1-(3-trifluoromethyl-phenyl)-ethyl]-urea;
1-Spiro[2.3]hex-5-yl-3-[1-(3-trifluoromethoxy-phenyl)-ethyl]-urea;
1-(3-Fluoro-5-trifluoromethyl-benzyl)-3-spiro[2.3]hex-5-yl-urea;
1-[2-Hydroxy-1-(3-trifluoromethyl-phenyl)-ethyl]-3-spiro[2.3]hex-5-yl-urea;
1-Spiro[2.3]hex-5-yl-3-{1-[6-(2,2,2-trifluoro-ethoxy)-pyrimidin-4-yl]-ethyl}-urea;
1-{1-[2-Methyl-6-(2,2,2-trifluoro-ethoxy)-pyrimidin-4-yl]-ethyl}-3-spiro[2.3]hex-5-yl-urea;
1-[2,2-Difluoro-1-(3-trifluoromethyl-phenyl)-ethyl]-3-spiro[2.3]hex-5-yl-urea;
1-[2-Methoxy-1-(3-trifluoromethyl-phenyl)-ethyl]-3-spiro[2.3] hex-5-yl-urea;
1-(4-Fluoro-3-trifluoromethyl-benzyl)-3-spiro[2.3]hex-5-yl-urea;
1-(2-Fluoro-3-trifluoromethyl-benzyl)-3-spiro[2.3]hex-5-yl-urea;
1-(2-Chloro-3-trifluoromethyl-benzyl)-3-spiro[2.3]hex-5-yl-urea;
1-(2-Fluoro-5-trifluoromethyl-benzyl)-3-spiro[2.3]hex-5-yl-urea;
1-{1-[5-Methoxy-6-(2,2,2-trifluoro-ethoxy)-pyrimidin-4-yl]-ethyl}-3-spiro[2.3]hex-5-yl-urea;
1-Spiro[2.3]hex-5-yl-3-(2-trifluoromethoxy-pyridin-4-ylmethyl)-urea;
1-{2-Methoxy-1-[2-(2,2,2-trifluoro-ethoxy)-pyridin-4-yl]-ethyl}-3-spiro[2.3]hex-5-yl-urea;
1-(3-Difluoromethoxy-benzyl)-3-spiro[2.3]hex-5-yl-urea;
1-Spiro[2.3]hex-5-yl-3-(3-trifluoromethoxy-benzyl)-urea;
1-Spiro[2.3]hex-5-yl-3-[3-(2,2,2-trifluoro-ethoxy)-benzyl]-urea;
1-(3-Difluoromethyl-benzyl)-3-spiro[2.3]hex-5-yl-urea;
1-Spiro[2.3]hex-5-yl-3-[2-(2,2,2-trifluoro-ethoxy)-pyridin-4-ylmethyl]-urea;
1-(6,6-Difluoro-spiro[3.3]hept-2-yl)-3-[2-(2,2,2-trifluoro-ethoxy)-pyridin-4-ylmethyl]-urea;
1-(6,6-Difluoro-spiro[3.3]hept-2-yl)-3-[1-(3-trifluoromethyl-phenyl)-ethyl]-urea;
1-Spiro[3.3]hept-2-yl-3-[(1H-[1,2,3]triazol-4-yl)-(3-trifluoromethyl-phenyl)-methyl]-urea;
1-[(1-Methyl-1H-tetrazol-5-yl)-(3-trifluoromethyl-phenyl)-methyl]-3-spiro[3.3]hept-2-yl-urea;
1-[(2-Methyl-2H-tetrazol-5-yl)-(3-trifluoromethyl-phenyl)-methyl]-3-spiro[3.3]hept-2-yl-urea;
1-[(3-Methyl-3H-[1,2,3]triazol-4-yl)-(3-trifluoromethyl-phenyl)-methyl]-3-spiro[3.3]hept-2-yl-urea;
1-[1-(2-Difluoromethoxy-pyridin-4-yl)-ethyl]-3-(6-iodo-spiro[3.3]hept-2-yl)-urea;
1-[(R)-1-(2-Difluoromethoxy-pyridin-4-yl)-ethyl]-3-spiro[3.3]hept-2-yl-urea;
1-[(S)-1-(2-Difluoromethoxy-pyridin-4-yl)-ethyl]-3-spiro[3.3]hept-2-yl-urea;
1-Spiro[3.3]hept-2-yl-3-{1-[3-((R)-2,2,2-trifluoro-1-methyl-ethoxy)-phenyl]-cyclopropyl}-urea;
1-Spiro[3.3]hept-2-yl-3-{1-[3-((S)-2,2,2-trifluoro-1-methyl-ethoxy)-phenyl]-cyclopropyl}-urea;
1-Spiro[3.3]hept-2-yl-3-[2-((R)-2,2,2-trifluoro-1-methoxymethyl-ethoxy)-pyridin-4-ylmethyl]-urea;
1-Spiro[3.3]hept-2-yl-3-[2-((S)-2,2,2-trifluoro-1-methoxymethyl-ethoxy)-pyridin-4-ylmethyl]-urea;
1-[(S)-1-(3-Difluoromethoxy-phenyl)-ethyl]-3-spiro[3.3]hept-2-yl-urea;
1-[(R)-1-(3-Difluoromethoxy-phenyl)-ethyl]-3-spiro[3.3] hept-2-yl-urea;
1-Spiro[3.3]hept-2-yl-3-[(S)-1-(3-trifluoromethyl-phenyl)-ethyl]-urea;
1-Spiro[3.3]hept-2-yl-3-{(S)-1-[3-(2,2,2-trifluoro-ethoxy)-phenyl]-ethyl}-urea;
1-{(S)-1-[4-Fluoro-3-(2,2,2-trifluoro-ethoxy)-phenyl]-ethyl}-3-spiro[3.3]hept-2-yl-urea;
1-Spiro[3.3]hept-2-yl-3-[(R)-2,2,2-trifluoro-1-(3-trifluoromethyl-phenyl)-ethyl]-urea;
1-Spiro[3.3]hept-2-yl-3-[(R)-(1H-[1,2,4]triazol-3-yl)-(3-trifluoromethyl-phenyl)-methyl]-urea;
1-Spiro[3.3]hept-2-yl-3-[2-((R)-2,2,2-trifluoro-1-methyl-ethoxy)-pyrimidin-4-ylmethyl]-urea;
1-Spiro[3.3]hept-2-yl-3-[2-((S)-2,2,2-trifluoro-1-methyl-ethoxy)-pyrimidin-4-ylmethyl]-urea;
1-Spiro[3.3]hept-2-yl-3-{(S)-1-[6-(2,2,2-trifluoro-ethoxy)-pyrimidin-4-yl]-ethyl}-urea;
1-Spiro[3.3]hept-2-yl-3-{(R)-1-[6-(2,2,2-trifluoro-ethoxy)-pyrimidin-4-yl]-ethyl}-urea;
1-[(R)-2-Hydroxy-1-(3-trifluoromethyl-phenyl)-ethyl]-3-spiro[3.3]hept-2-yl-urea;
1-[(S)-2-Hydroxy-1-(3-trifluoromethyl-phenyl)-ethyl]-3-spiro[3.3]hept-2-yl-urea;
1-[(R)-2-Hydroxy-1-(3-trifluoromethyl-phenyl)-ethyl]-3-spiro[2.3]hex-5-yl-urea;
1-[(R)-1-(2-Difluoromethoxy-pyridin-4-yl)-2-methoxy-ethyl]-3-spiro[3.3]hept-2-yl-urea;
1-{(R)-2-Methoxy-1-[2-(2,2,2-trifluoro-ethoxy)-pyridin-4-yl]-ethyl}-3-spiro[2.3] hex-5-yl-urea;
1-[(S)-1-(2-Difluoromethoxy-pyridin-4-yl)-ethyl]-3-spiro[2.3]hex-5-yl-urea; or
1-[(S)-1-(2-Bromo-5-trifluoromethyl-phenyl)-ethyl]-3-spiro[3.3] hept-2-yl-urea;
or a salt thereof.

12. A pharmaceutical composition comprising, as active principle, a compound according to claim 1, or a pharmaceutically acceptable salt thereof, and at least one therapeutically inert excipient.

13. (canceled)

14. A method for the prevention or treatment of a disease selected from epilepsy, myokymia, tinnitus, neuropathic and inflammatory pain, psychiatric disorders, and diseases affecting the smooth muscles in a patient comprising administration to said patient of a pharmaceutically active amount of a compound according to claim 1, or a pharmaceutically acceptable salt thereof.

15. (canceled)

16. A pharmaceutical composition comprising, as active principle, a compound according to claim 11, or a pharmaceutically acceptable salt thereof, and at least one therapeutically inert excipient.

17. A method for the prevention or treatment of a disease selected from epilepsy, myokymia, tinnitus, neuropathic and inflammatory pain, psychiatric disorders, and diseases affecting the smooth muscles in a patient comprising administration to said patient of a pharmaceutically active amount of a compound according to claim 11, or a pharmaceutically acceptable salt thereof.

18. A compound according to claim 3, wherein R^{2A} represents hydrogen, methyl, or hydroxymethyl;
and R^{2B} represents hydrogen;
or a salt thereof.

19. A compound according to claim 18, wherein R³ represents a fragment 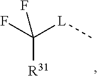
wherein
R³¹ represents hydrogen, or fluoro; and
L represents a direct bond, —CHR^{L}—O—*, —O—CH₂—*, —CH₂—NH—*, —CH₂—N(CH₃)*, or —O—; wherein R^{L} represents hydrogen, methyl, CH₃—O—CH₂—, or (CH₃)₂NCH₂—; wherein the asterisks indicate the bond which is linked to the aromatic carbon atom;
or a salt thereof.

20. A compound according to claim 18, wherein the fragment 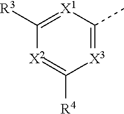
represents: 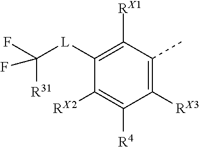
wherein R^{X1} represents hydrogen, fluoro, chloro, methyl, or methoxy; R^{X2} represents hydrogen; R^{X3} represents hydrogen, fluoro, or chloro; R⁴ represents hydrogen, fluoro, chloro, or methyl; R³¹ represents hydrogen, or fluoro; and L represents a direct bond, —CHR^{L}—O—*, —CH₂—NH—*, —CH₂—N(CH₃)—*, —O—, or; wherein R^{L} represents hydrogen, or methyl; wherein the asterisks indicate the bond which is linked to the aromatic carbon atom; or 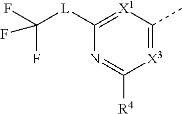
wherein
X³ represents nitrogen and X¹ represents CR^{X1};
X¹ represents nitrogen and X³ represents CR^{X3}; or
X¹ represents CR^{X1} and X³ represents CR^{X3};
wherein R^{X1} represents hydrogen, or methoxy; R^{X3} represents hydrogen; R⁴ represents hydrogen, or methyl; and L represents —CHR^{L}—O*, —CH₂—NH—*, or —CH₂—N(CH₃)—*; wherein R^{L} represents hydrogen, methyl, CH₃—O—CH₂—, or (CH₃)₂NCH₂—; wherein the asterisks indicate the bond which is linked to the aromatic carbon atom;
or a salt thereof.
